(19) United States
(12) Patent Application Publication
(10) Pub. No.: US 2024/0033283 A1
(43) Pub. Date: Feb. 1, 2024

(54) FUNCTIONALIZED AND CROSSLINKED POLYMERS

(71) Applicant: PMIDG, LLC, Anderson, SC (US)

(72) Inventors: David M. GRAVETT, Anderson, SC (US); Kara Bethany ACAMPORA, Anderson, SC (US); Prabhjot SAINI, Anderson, SC (US)

(21) Appl. No.: 18/265,111

(22) PCT Filed: Dec. 2, 2021
(86) PCT No.: PCT/US21/61675

§ 371 Date: Jun. 2, 2023

Related U.S. Application Data
(60) Provisional application No. 63/121,037, filed on Dec. 3, 2020.

(51) Int. Cl. A61K 31/728; C08L 5/08; C08B 37/08; A61K 9/06; A61K 31/167; A61K 38/18; A61K 38/48; A61K 31/704; A61K 33/243; A61K 31/7068; A61K 31/282; A61L 27/20; A61L 24/08; A61L 31/04

(52) U.S. Cl. CPC A61K 31/728; C08L 5/08; C08B 37/0072; A61K 9/06; A61K 31/167; A61K 38/1875; A61K 38/4893; A61K 31/704; A61K 33/243; A61K 31/7068; A61K 31/282; A61L 27/20; A61L 24/08; A61L 31/042; A61L 2300/424; A61L 2430/34; A61L 2300/402; A61L 2300/204; A61L 2300/236; A61L 2400/06

(57) ABSTRACT

Disclosed herein are compositions and methods of making and using such compositions. Polyhydric polymers may be converted to derivatives thereof by reaction with divinyl sulfone to provide vinyl sulfone substituted polymers, where the polymers may additionally be crosslinked, and the crosslinked and non-crosslinked derivatives may be used in biomedical and other applications.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### TECHNICAL FIELD

The present disclosure comprises compositions comprising functionalized hyaluronic acid, including crosslinked functionalized hyaluronic acid, and methods for preparation and uses of such compositions.

### BACKGROUND OF THE DISCLOSURE

Hyaluronic acid, abbreviated HA; conjugate base hyaluronate, is also called hyaluronan, and is an anionic, nonsulfated glycosaminoglycan distributed widely throughout connective, epithelial, and neural tissues. It is unique among glycosaminoglycans as it is nonsulfated, forms in the plasma membrane instead of the Golgi apparatus, and can be very large (molecular weight): human synovial HA averages about 7 million Da per molecule, or about 20,000 disaccharide monomers, [4] while other sources mention 3-4 million Da. [5]

As one of the chief components of the extracellular matrix, HA contributes significantly to cell proliferation and migration. The average 70 kg (150 lb) person has roughly 15 grams of hyaluronan in the body, one-third of which is turned over (i.e., degraded and synthesized) per day.

Hyaluronic acid (HA) is a general humectant, a substance that retains moisture, and is capable of binding over one thousand times its weight in water. HA is found in many areas of the human body, including the skin, eyes, and synovial fluid of the joints. HA used in beauty and skincare products is primarily made by bacteria in a lab via a process called biofermentation. HA is currently widely used in cosmetics and skin treatments because, in aging, the production of substances in the skin, including hyaluronic acid (along with collagen and elastin) decreases. As a result, human skin loses volume, hydration, and plumpness. Many skincare products containing HA claim to increase hydration within the skin. Larger molecular weight HA molecules, despite being the best at binding water and offering hydration, cannot penetrate into the skin. When applied topically (to the skin), these molecules sit on top of the skin, offering hydration only at the very surface. Smaller molecular weight HA molecules, which bind less water than larger HA molecules, can penetrate deeper into the skin (though it is thought to only penetrate into the epidermis, the topmost layer of skin).

HA is also used in products other than skin care, such as for dermal fillers, an injectable form of HA, and for wound care. As noted above, HA is naturally degraded by the body, which limits the effectiveness of HA for treatments. What is needed are compositions comprising derivatized or functionalized HA, compositions comprising cross-linked derivatized or functionalized HA, and methods of making and using such compositions.

### SUMMARY

In brief, the present disclosure provides hyaluronic acid derivatives, crosslinked forms of hyaluronic acid derivatives, methods of making derivatives, methods of crosslinking hyaluronic acid derivatives, formulations of HA derivatives and crosslinked forms thereof and methods of using such compositions and HA derivative polymers.

For example, in an aspect, the present disclosure comprises a derivative of hyaluronic acid, in which one or more hydroxyl groups of the hyaluronic acid is a modified hydroxyl group, wherein the derivative of hyaluronic acid has the structure HA-(OCH₂CH₂SO₂CH₂CH₂—Ar—Y)ₙ where HA is hyaluronic acid, X is S or NH, Ar is a benzene ring, Y is a carboxylic acid group or a hydrogen and n is the number of modified hydroxyl groups where n is an integer and n≥1 wherein derivatives of hyaluronic acid has a slower degradation rate than non-derivatized hyaluronic acid when exposed to hyaluronidase under the same in vitro conditions.

In an aspect, the present disclosure comprises a derivative of hyaluronic acid, in which two or more hydroxyl groups of the hyaluronic acid are modified hydroxyl groups, wherein the derivative of hyaluronic acid has the structure (CH₂═CH—SO₂CH₂CH₂O)ₘ—HA-(OCH₂CH₂SO₂CH₂CH₂—X-AR—Y)ₙ where HA is hyaluronic acid, X is S or NH, Ar is a benzene ring, Y is a carboxylic acid group or a hydrogen, each of n and m is an integer, and n≥1 and m≥1 and wherein the derivatized hyaluronic acid polymers of the present disclosure have a slower degradation rate than non-derivatized hyaluronic acid when exposed to hyaluronidase under the same in vitro conditions.

In an aspect, the present disclosure comprises derivatives of hyaluronic acid such as described above, which comprise one or more of the following:
1) The derivative wherein 0.25-50% of a sum of the hydroxyl groups and the modified hydroxyl groups are a modified hydroxyl group.
2) The derivative wherein Y is in an ortho position.
3) The derivative wherein Y is in a meta position.
4) The derivative wherein Y is in a para position.
5) the derivatized hyaluronic acid polymer has a slower degradation rate than non-derivatized hyaluronic acid when exposed to hyaluronidase under the same in vitro conditions.

In an aspect, the present disclosure provides a crosslinked hyaluronic acid derivative comprising a reaction product of a derivative of a hyaluronic acid, and a crosslinking agent, wherein
a) the derivative of hyaluronic acid has the structure HA-(OCH₂CH₂SO₂CH₂CH₂—X—Ar—Y)ₙ wherein one or more hydroxyl groups of the hyaluronic acid is a modified hydroxyl group, and wherein HA is hyaluronic acid, comprising hydroxyl groups, X is S or NH, Ar is a benzene ring, Y is a carboxylic acid group or a hydrogen and n is the number of modified hydroxyl groups where n 1; and
b) the crosslinking agent comprises at least two functional groups that are capable of reacting with the hydroxyl groups or carboxylic acid groups of the derivative of hyaluronic acid.

In an aspect, the crosslinked polymer may comprise one or more of the following:
i) The crosslinked polymer wherein 0.25-50% of a sum of the hydroxyl groups and the modified hydroxyl groups are a modified hydroxyl group.
ii) The crosslinked polymer wherein Y is in an ortho position
iii) The crosslinked polymer wherein Y is in a meta position.
iv) The crosslinked polymer wherein Y is in a para position and
v) the crosslinked derivatized hyaluronic acid polymers of the present disclosure have a slower degradation rate than crosslinked non-derivatized hyaluronic acid when exposed to hyaluronidase under the same in vitro conditions.

In an aspect, the present disclosure provides a crosslinked hyaluronic acid derivative comprising a reaction product of a derivative of a hyaluronic acid with itself, wherein the derivative of hyaluronic acid has the structure HA-(OCH₂CH₂SO₂CH₂CH₂—X—Ar—Y)ₙ wherein one or more hydroxyl groups of the hyaluronic acid is a modified hydroxyl group, and wherein HA is hyaluronic acid, comprising hydroxyl groups, X is S or NH, Ar is a benzene ring, Y is a carboxylic acid group of a hydrogen and n is the number of modified hydroxyl groups where n≥1. In an aspect, the crosslinked polymer may comprise one or more of the following:
i) The crosslinked polymer wherein 0.25-50% of a sum of the hydroxyl groups and the modified hydroxyl groups are a modified hydroxyl group;
ii) The crosslinked polymer wherein Y is in an ortho position;
iii) The crosslinked polymer wherein Y is in a meta position;
iv) The crosslinked polymer wherein Y is in a para position; and/or
v) the crosslinked derivatized hyaluronic acid polymer of the present disclosure has a slower degradation rate than crosslinked non-derivatized hyaluronic acid when exposed to hyaluronidase under the same in vitro conditions.

In an aspect, the present disclosure comprises a crosslinked polymer comprising a reaction product of a derivative of hyaluronic acid, and a crosslinking agent, wherein the derivative of hyaluronic acid comprises vinyl groups and has the structure (CH₂═CH—SO₂CH₂CH₂O)ₘ—HA-(OCH₂CH₂S₀ZCH₂CH₂—X—Ar—Y)ₙ wherein two or more hydroxyl groups of the hyaluronic acid are modified hydroxyl groups, HA is hyaluronic acid comprising hydroxyl groups, X is S or NH, Ar is a benzene ring, Y is a carboxylic acid group or a hydrogen, n≥1 and m≥1; and the crosslinking agent comprises at least two functional groups that are capable of reacting with the vinyl groups of the derivative of hyaluronic acid.

In an aspect, the crosslinked polymer may comprise one or more of the following:
i) The crosslinked polymer wherein 0.25-50% of a sum of the hydroxyl groups and the modified hydroxyl groups are a modified hydroxyl group.
ii) The crosslinked polymer wherein Y is in an ortho position.
iii) The crosslinked polymer wherein Y is in a meta position.
iv) The crosslinked polymer wherein Y is in a para position.
v) the crosslinked derivatized hyaluronic acid polymer has a slower degradation rate than crosslinked non-derivatized hyaluronic acid when exposed to hyaluronidase under the same in vitro conditions.

In an aspect, the present disclosure provides a crosslinked polymer comprising a reaction product of a derivative of hyaluronic acid with itself, wherein the derivative of hyaluronic acid comprises vinyl groups and has the structure (CH₂═CH—SO₂CH₂CH₂O)ₘ—HA-(OCH₂CH₂SO₂CH₂CH₂—X—Ar—Y)ₙ wherein two or more hydroxyl groups of the hyaluronic acid are modified hydroxyl groups, HA is hyaluronic acid comprising hydroxyl groups, X is S or NH, Ar is a benzene ring, Y is a carboxylic acid group or a hydrogen, n≥1 and m≥1.

In an aspect, the crosslinked polymer may comprise one or more of the following:
i) The crosslinked polymer wherein 0.25-50% of a sum of the hydroxyl groups and the modified hydroxyl groups are a modified hydroxyl group.
ii) The crosslinked polymer wherein Y is in an ortho position.
iii) The crosslinked polymer wherein Y is in a meta position.
iv) The crosslinked polymer wherein Y is in a para position.
v) the crosslinked derivatized hyaluronic acid polymer has a slower degradation rate than crosslinked non-derivatized hyaluronic acid when exposed to hyaluronidase under the same in vitro conditions.

In an aspect, the present disclosure provides a process comprising:
a) reacting hydroxyl groups attached to hyaluronic acid (HA) with divinyl sulfone (DVS) to provide a first derivative of the polymer; and
b) reacting the first derivative of the polymer with a nucleophile of a formula selected from X—Ar—Y to provide a second derivative of the polymer;
wherein Ar is a benzene ring, X is a nucleophilic group, and Y is selected from carboxylic acid, and a hydrogen. In an aspect, the process may comprise one or more of the following:
1) The process wherein 0.25-50% of the hydroxyl groups present on the polymer are converted to oxyethyl ethenyl sulfone groups of the formula —OCH₂CH₂—SO₂CH═CH₂.
2) The process wherein the polymer is hyaluronic acid and the first derivative of the polymer is an oxyethyl ethenyl sulfone derivative of the hyaluronic acid=HA-OCH₂CH₂SO₂CH═CH₂ (HA-DVS).
3) The process wherein the second derivative is HA-OCH₂CH₂SO₂CH₂CH₂—X—Ar—Y (HA-DVS-ar-Y).
4) The process wherein 0.25-50% of the hydroxyl groups present on the polymer are converted to —OCH₂CH₂SO₂CH₂CH₂—X—Ar—Y groups.
5) The process wherein X′ is thiol and X is —S—.
6) The process further comprising reacting the second derivative of the polymer with a crosslinking agent to provide a third derivative of the polymer, where the third derivative is a crosslinked polymer.
7) The process further comprising reacting the second derivative of the polymer with itself to provide a third derivative of the polymer, where the third derivative is a crosslinked polymer.

In an aspect, the present disclosure provides a derivative of hyaluronic acid prepared by any of the processes identified herein. In an aspect, the present disclosure provides a crosslinked polymer prepared by any of the processes identified herein.

In an aspect, the present disclosure provides a composition comprising a derivative of hyaluronic acid, as described herein, where the composition also comprises an excipient. In an aspect, the present disclosure provides a composition comprising a crosslinked derivative of hyaluronic acid, as described herein, where the composition also comprises an excipient. Compositions disclosed herein may optionally include one or more of a synthetic polymer, thermosreversible polymer, biodegradable polymer, a polysaccharide, a buffer, a complexing agent, a tonicity modulator, an ionic strength modifier, a solvent, an anti-oxidant, a preservative, a viscosity modifier, a pH modifier, a surfactant, an emulsifier, a phospholipid, a stabilizer and/or a porogen. Also optionally, a composition as disclosed herein may further comprise a biologically active agent.

In an aspect, the present disclosure provides methods of using the HA polymers and compositions as disclosed herein. For example, the present disclosure provides the following aspects:
1) A method for wound healing comprising administering to a subject in need thereof an effective amount of a composition as described herein.
2) A bulking agent comprising a composition as described herein.
3) A dermal filler comprising a composition as described herein.
4) A method of filling a void in a subject in need thereof comprising administering to the subject a dermal filler as described herein.
5) A viscosupplement comprising a composition as described herein.
6) A method of relieving joint pain in a subject in need thereof, comprising administering to the subject a viscosupplement as described herein.
7) A method of preventing surgical adhesions in a subject in need thereof comprising administering the subject an effective amount of a composition as described herein.
8) A tissue sealant comprising a composition as described herein.
9) A method of sealing tissue in a subject in need thereof comprising administering to the subject an effective amount of a tissue sealant as described herein.
10) A method of treating bacterial vaginosis in a subject in need thereof comprising administering to the subject an effective amount of a composition as described herein.
11) An eye drop composition comprising a composition as described herein.
12) An ocular bandage comprising a composition as described herein.
13) A method of treating an ocular condition in a subject in need thereof comprising administering the subject an effective amount of a composition as described herein.
14) A punctal plug comprising a composition as described herein.
15) A method of treating mucositis in a subject in need thereof comprising administering to the subject an effective amount of a composition as described herein.
16) An anti-bacterial formulation comprising a composition as described herein.
17) A method of treating an ear condition comprising administering to a subject in need thereof an effective amount of a composition as described herein.
18) A method of drug delivery to a subject in need thereof comprising administering to the subject an effective amount of a composition as described herein that comprises the drug.
19) A biopsy plug comprising a composition as described herein.
20) A plug for female sterilization comprising a composition as described herein.
21) A tissue scaffold comprising a composition as described herein.
22) The method of supporting tissue growth in a subject in need thereof comprising implanting in the subject a tissue scaffold as described herein.
23) A burr hole plug comprising a composition as described herein.
24) A nerve guide comprising a composition as described herein.
25) A vaginal lubricant comprising a composition as described herein.
26) A coating for a device comprising a composition as described herein.
27) A method for coating a device comprising applying a coating as described herein onto a surface of the device.
28) A method for additive manufacturing comprising photocuring a derivative of a hyaluronic acid as described herein or prepared by a process as described herein, to provide a crosslinked article formed by additive manufacturing.
29) A tissue spacer comprising a composition as described herein.

The above-mentioned and additional features of the present disclosure and the manner of obtaining them will become apparent, and the disclosure will be best understood by reference to the following more detailed description. All references disclosed herein are hereby incorporated by reference in their entirety as if each was incorporated individually.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more aspects are set forth in the description below. The features illustrated or described in connection with one exemplary aspect may be combined with the features of other aspects. Thus, any of the various aspects described herein can be combined to provide an aspect. Aspects of the aspects can be modified, if necessary, to employ concepts of the various patents, applications and publications as identified herein to provide yet an aspect. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various aspects. Non-limiting and non-exhaustive aspects are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more aspects are described hereinafter with reference to the accompanying drawings in which:

FIG. 1 shows the storage modulus (G′) and the loss modulus (G″) against angular frequency for a solution of a 2-mercaptobenzoic acid derivative (80% substitution) of hyaluronic acid according to the present disclosure.

FIG. 2 show exemplary reactions, A-P, of the present disclosure.

FIG. 3 show exemplary reactions, A-H, of the present disclosure.

FIG. 4 shows the change is solution viscosity of various derivatives of the present disclosure relative to that of hyaluronic acid when exposed to hyaluronidase degradation.

FIG. 5 shows the weight change (%) of BDDE (1,4-butanediol diglycidyl ether) crosslinked gels of the present disclosure relative to a similarly crosslinked gel of hyaluronic acid.

### DETAILED DESCRIPTION OF THE DISCLOSURE

The present disclosure may be understood more readily by reference to the following detailed description of preferred aspects of the disclosure and the Examples included herein.

In one aspect, the present disclosure provides derivatized hyaluronic acid polymers, including crosslinked versions thereof, that have a degradation rate that is slower than that of unmodified hyaluronic acid when exposed to hyaluronidase under similar in vitro conditions.

Though not wishing to be bound by any particular theory, it is believed the derivatized hyaluronic acid polymers of the present disclosure provide useful properties that are not available from non-derivatized hyaluronic acid polymers currently available. It was unexpectedly found that derivatized hyaluronic acid polymers of the present disclosure have a slower degradation rate than non-derivatized hyaluronic acid when exposed to hyaluronidase under the same in vitro conditions.

In an aspect the present disclosure utilizes a hyaluronic acid with one or more available hydroxyl groups and reacts those hydroxyl groups under specific conditions as disclosed herein with divinyl sulfone such that only one of the vinyl groups of the divinyl sulfone reacts with an hydroxyl group via an addition reaction to form an ether bond between the hyaluronic acid and the residue of the divinyl sulfone. The degree of reaction ranges from about 0.5% to about 50% of the available hydroxyl groups on the hyaluronic acid. At higher substitution, i.e., around 50%, some degree of crosslinking will typically occur. Thus, the present disclosure provides vinyl sulfone substituted hyaluronic acid with minimal to no crosslinking, or a hyaluronic acid that have a level of vinyl sulfone substitution along with a level of crosslinking due to double reaction of the divinyl sulfone (DVS) (i.e., reaction of both ethenyl groups of the DVS with hydroxyl groups).

The residual vinyl group of the vinyl sulfone can be then reacted with a compound that has a reactive thiol group. This reaction occurs via a Michael addition between the residual vinyl group of the divinyl sulfone and the free thiol group such that a thioether bond is formed. There are numerous variations of the degree of substitution, the thiol derivative used, the sequence of the reactions and the replication of reactions that provide a large variety of compositions. These compositions can then be crosslinked in many different ways. Compositions made by such methods can be used for numerous medical and non-medical applications.

The functionalized polymers of the present disclosure are prepared as described herein. Typically, a hyaluronic acid having hydroxyl groups is combined with divinyl sulfone (DVS) under suitable reaction conditions. Those reaction conditions include a suitable pH of the solution, where the reaction typically occurs under basic conditions, e.g., a pH of 11-14, or 12-13, e.g., about 12.5. The reaction conditions include a suitable solvent, where water or DMSO are suitable solvents, e.g., the reaction may be conducted in water. The description of reaction conditions may further include stirring the reacting mixture, e.g., stirring with a stirring rate of >200 rpm (rotations per minute), such as 250-750 rpm. Furthermore, the description of reaction conditions may include specification of the relative amounts of DVS and hyaluronic acid that are combined, where these relative amounts may be expressed in terms of moles of DVS to moles of repeat disaccharide unit in the hyaluronic acid. For instance, a method for preparing derivatized hyaluronic acid polymer may be described in terms of the ratio of DVS:hyaluronic acid repeat unit, where this ratio may be at least 0.5:1, e.g., up to about 5:1, or up to about 7.5:1, or up to about 10:1, or up to about 15:1, or up to about 20:1

An exemplary functionalized polymer is hyaluronic acid (HA). HA is a polysaccharide illustrated by the structure shown below.

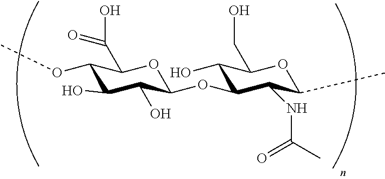

HA contains two different functional groups, namely hydroxyl groups and carboxylic acid groups. HA also contains ether and acetamide groups, however these are essentially chemically inert. In commercially available preparations of HA, some or all of the carboxylic acids may be present as the corresponding salt, e.g., as the sodium, potassium or ammonium salt. In the present disclosure, and unless the context indicates otherwise, HA refers inclusively to polymers of the structure shown above as well as the corresponding carboxylate salts of those polymers.

In an aspect, the present disclosure provides a process wherein a hyaluronic acid that has an available hydroxyl group, i.e., a hydroxyl group that is capable of undergoing a reaction with divinyl sulfone, is reacted with DVS under basic conditions. If the conditions are selected appropriately, such as disclosed herein, the reaction can be controlled such that one of the vinyl groups of the divinyl sulfone will react with a free hydroxyl group of the hyaluronic acid such than hyaluronic acid does not crosslink to such an extent that it forms a hydrogel. This results in the hyaluronic acid being functionalized with the divinyl sulfone such that one of the vinyl groups undergoes reaction with a hydroxyl group of the hyaluronic acid and the other vinyl group remains functional. The vinyl group of the divinyl sulfone reacts with the hydroxyl group by an addition reaction that results in an ether linkage.

The reaction may be performed under basic conditions with a pH of greater than 11. Optionally, the pH is in the range of 12.0 to 13.5. Optionally, the pH range is in the 12.2 to 13.1 range. Optionally, the pH range is in the 12.2 to 12.6 range.

To ensure that the predominant reaction is a single reaction of one of the vinyl groups of the divinyl sulfone, and not a crosslinking reaction in which predominantly both the vinyl groups react with hydroxyl groups of the polysaccharide to form a crosslinked gel, the molar ratio of the divinyl sulfone to that of the polysaccharide repeat units is preferably greater than 1. In an aspect, the molar ratio of the divinyl sulfone to that of the polysaccharide repeat units is greater than 5. In an aspect, the molar ratio of the divinyl sulfone to that of the polysaccharide repeat units is greater than 7. In an aspect, the molar ratio of the divinyl sulfone to that of the polysaccharide repeat units is greater than 10. In an aspect, the molar ratio of the divinyl sulfone to that of the polysaccharide repeat units is greater than 15.

To provide intimate contact between the reactants, the reaction mixture may be stirred. In order to ensure that there is adequate stirring of the reaction solution during the reaction, the rotational speed of the mixing impellor should be controlled. In an aspect, the revolutions per minute (rpm) of the mixing impellor should be in the range of 200 to 400 rpm. In an aspect, the revolutions per minute (rpm) of the mixing impellor should be in the range of 400 to 600 rpm. In an aspect, the revolutions per minute (rpm) of the mixing impellor should be in the range of 600 to 800 rpm.

The amount of substitution accomplished may be controlled, in part, by the duration of exposure of hyaluronic acid to divinyl sulfone at a pH of greater than 11 (reaction time). In an aspect, the reaction time can range from 10 seconds through to 60 minutes. In an aspect, the reaction time can be in the range of 2 minutes to 35 minutes. In an aspect, the reaction time can be in the range of 4 minutes to 30 minutes.

The solvent used for the reaction can be water, water with an ionic modifier, for example NaCl, a combination of water and a water-miscible solvent. Water miscible solvents can include but are not limited to methanol, ethanol, isopropanol, dimethyl formamide (DMF), acetone, 1,4-dioxane, pyridine, dimethyl sulfoxide (DMSO), tetrahydrofuran (THF) and acetonitrile.

The temperature of the reaction mixture can also be used to influence the amount of substitution of the polysaccharide by the divinyl sulfone. In an aspect, the reaction mixture can be maintained at a temperature that is lower than 25° C. so as to reduce the rate of the reaction. This can enable lower substitution levels for the same duration as compared to room temperature or it can allow for a longer reaction time that that at room temperature to yield a similar amount of substitution. In an aspect, the temperature can be in the 15° C. to 20° C. range. In an aspect, the reaction mixture can be in the 10° C. to 15° C. range. In yet an aspect, the temperature can be in the 2° C. to 10° C. range. In an aspect, the temperature can be increase above 25° C. so as to provide shorter reaction times as compared to 25° C. to get similar amounts of substitution or to get greater substitution as compared to 25° C. for an equivalent amount of reaction time. In an aspect, the reaction mixture can be in the 26° C. to 35° C. range. In an aspect, the reaction mixture can be in the 36° C. to 50° C. range. In an aspect, the reaction mixture can be in the 51° C. to 75° C. range.

The amount of substitution, as measured by the molar ratio of the attached vinyl group from the divinyl sulfone to the hyaluronic acid repeat unit, can be greater than 5%. In an aspect, for hyaluronic acid with at least one hydroxyl group, the amount of substitution is in the range of 5% to 35%. In an aspect, for hyaluronic acid with at least one hydroxyl group, the amount of substitution is in the range of 36% to 70% range. In an aspect, for hyaluronic acid with at least one hydroxyl group, the amount of substitution is in the range of 71% to 100% range. In an aspect, for hyaluronic acid with at least two hydroxyl groups, the amount of substitution is in the range of 101% to 200% range.

In an aspect, hyaluronic acid that comprise at least one hydroxyl group that is available for reaction with divinyl sulfone under conditions where the pH is greater than 12 is suitable for use in this disclosure. The hyaluronic acid includes hyaluronic acid salts such as sodium hyaluronate. an aspect

The molecular weight of the hyaluronic acid can be selected. Molecular weights from 1,000 to 5,000,000 may be used. In an aspect, the polysaccharide has a molecular weight of over 1,000. In an aspect, the polysaccharide has a molecular weight in the range of 1,000 to 50,000. In an aspect, the polysaccharide has a molecular weight in the range of 50,000 to 200,000. In an aspect, the polysaccharide has a molecular weight in the range of 200,000 to 600,000. In yet an aspect, the polysaccharide has a molecular weight in the range of 600,000 to 1,000,000. In yet an aspect, the polysaccharide has a molecular weight in the range of 1,000,000 to 2,500,000. In yet an aspect, the polysaccharide has a molecular weight in the range of 2,500,000 to 5,000,000. The molecular weight can be measured gel permeation chromatography or intrinsic viscosity. Optionally, the intrinsic viscosity of the hyaluronic acid is in the range of 0.3 to 3 m³/Kg. In an aspect, the hyaluronic acid has an intrinsic viscosity of between 0.3 and 0.9 m³/Kg. In an aspect, the hyaluronic acid has an intrinsic viscosity of between 0.9 and 2.0 m³/Kg. In an aspect, the hyaluronic acid has an intrinsic viscosity of between 2.0 and 3.0 m³/Kg.

After hyaluronic acid has been reacted with DVS to create a first derivative of the polymer, this first derivative is then reacted with a nucleophile, e.g., a thiol derivative, of a formula selected from X′—Ar—Y to provide a second derivative of the polymer. In these formulae, Ar is a benzene ring, X′ is a nucleophilic group, and Y is selected from a hydrogen, carboxylic acid, sulfonic acid, amino and hydroxyl. The nucleophile contains a thiol group as X in a thiol derivative. Thiol compounds can include but are not limited to 2-mercaptobenzoic acid (thiosalicylic acid), 3-mercaptobenzoic acid, 4 mercaptobenzoic acid, 2-aminothiophenol, 3-aminothiophenol, 4-aminothiophenol, thiophenol, 2-mercaptophenol, 3-mercaptophenol, 4-mercaptophenol and salts thereof and are also identified as R₁SH herein. In an aspect, the thiol compound is 2-mercaptobenzoic acid.

In an aspect, a hyaluronic acid derivative is not crosslinked. In an aspect, the storage modulus (G′) of an aqueous solution of the hyaluronic acid derivative of the present disclosure is less than the loss modulus (G″) at rheological frequencies less than 1 hz.

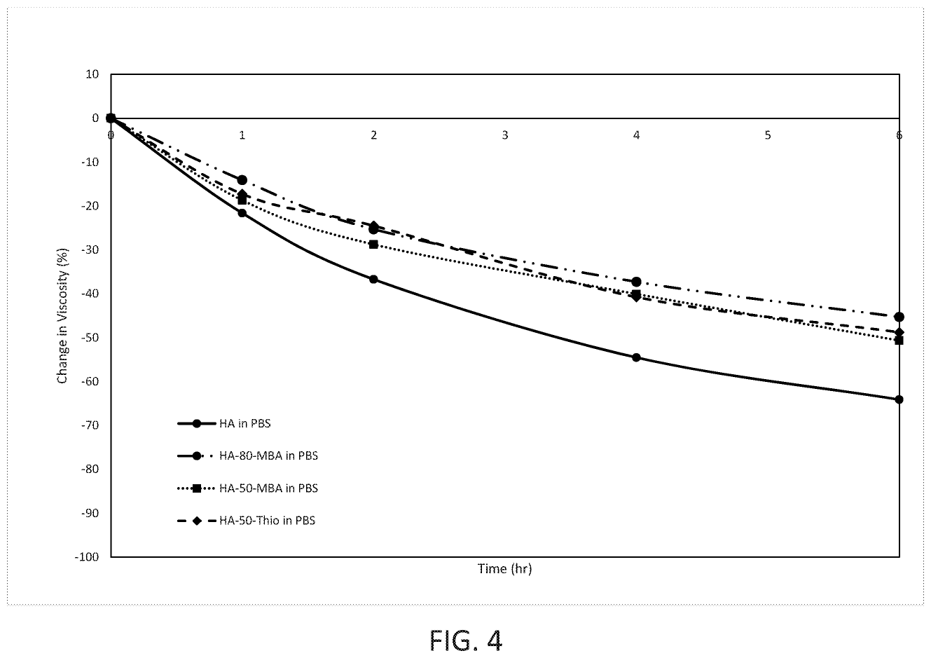

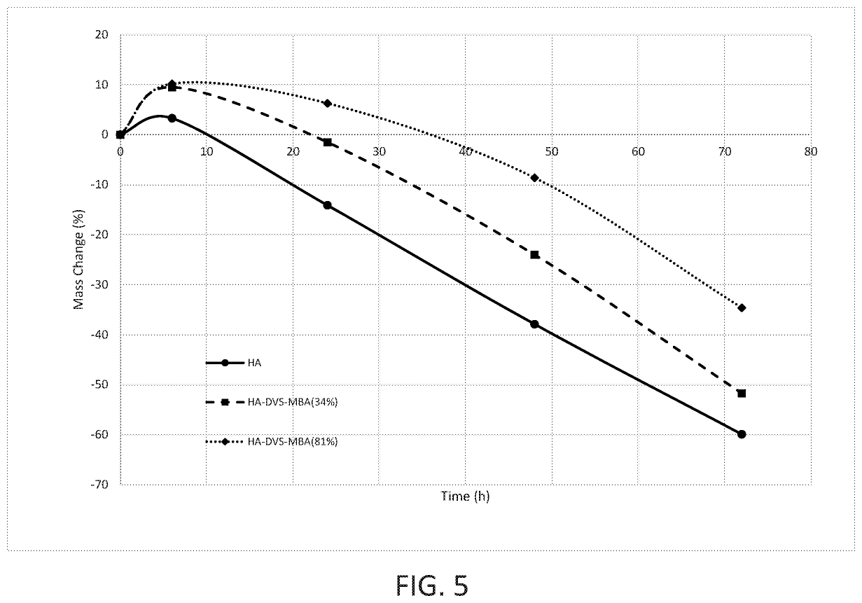

Derivatives of this disclosure have a slower degradation rate than non-derivatized hyaluronic acid when exposed to hyaluronidase under similar in vitro conditions. In an aspect, a solution of a derivative of this disclosure, have a slower degradation rate than non-derivatized hyaluronic acid when exposed to hyaluronidase under similar in vitro conditions. In an aspect, the crosslinked form of the derivatives of this disclosure have a slower degradation rate than non-derivatized hyaluronic acid when exposed to hyaluronidase under similar in vitro conditions. FIG. 4 shows that the 2-mercaptobenzoic acid derivative of hyaluronic acid and the thiophenol derivative of hyaluronic acid have less viscosity reduction over time as compared to a non-derivatized solution of hyaluronic acid when exposed to hyaluronidase. FIG. 5 shows that 1,4-butanediol diglycidyl ether (BDDE) crosslinked gel of 2-mercaptobenzoic acid derivative of hyaluronic acid has smaller mass loss over time as compared to a 1,4-butanediol diglycidyl ether (BDDE) crosslinked gel of non-derivatized hyaluronic acid.

In an aspect, a second thiol compound can be used to react with the residual vinyl groups of the derivative described herein. In an aspect, the second thiol compound can be reacted with the residual vinyl groups prior to use of the first thiol compound. In an aspect, the second thiol compound can be reacted with the residual vinyl groups simultaneously with the first thiol compound. In an aspect, the second thiol compound can be reacted with the residual vinyl groups after an initial reaction with the first thiol compound.

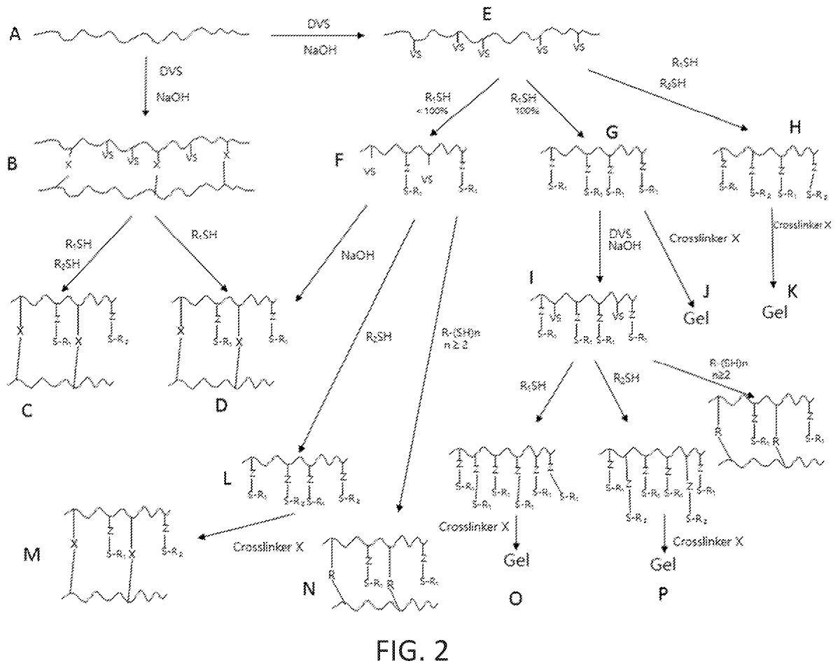

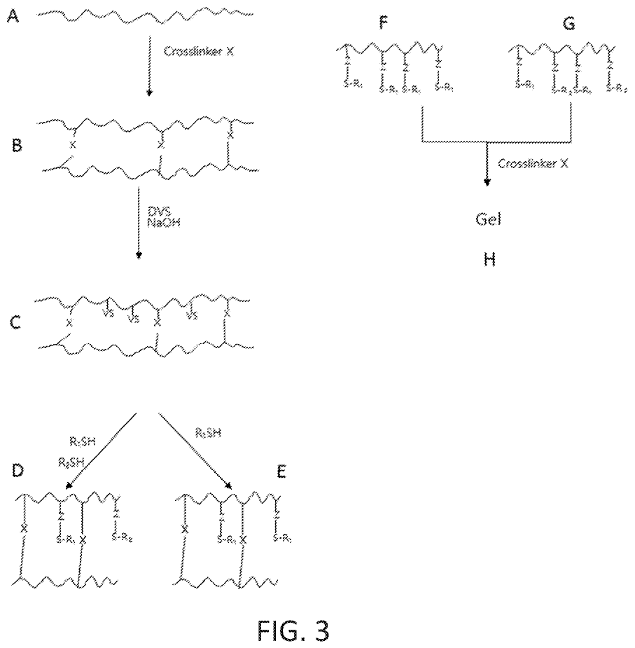

Second thiol compounds, identified by the formula R₂SH in FIGS. 2 and 3 can be used to prepare hyaluronic acid derivatives as disclosed herein. R₂ may be an aliphatic or aromatic moiety, either of which may have one or more substituents, e.g., be a substituted aliphatic moiety or a substituted aromatic moiety. An aliphatic moiety refers to an alkyl or cycloalkyl moiety, either having 1-20 carbon atoms.

“Alkyl” refers to a straight or branched hydrocarbon chain radical consisting solely of carbon and hydrogen atoms, containing no unsaturation, having from one to the specified number of carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. In an aspect the alkyl group has 1 carbon. In an aspect the alkyl group has 2 carbons. In an aspect the alkyl group has 3 carbons. In an aspect the alkyl group has 4 carbons. In an aspect the alkyl group has 4 carbons. In an aspect the alkyl group has 5 carbons. In an aspect the alkyl group has 6 carbons. Two or more of these aspects may be combined to describe derivatives of the disclosure.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically herein, a cycloalkyl group may be optionally substituted by one or more substituents independently selected at each occurrence.

An aromatic moiety refers to a carbocyclic aromatic moiety, a.k.a., an aryl moiety, or a heteroaromatic moiety, a.k.a., a heteroaryl moiety, either having 1-20 carbon atoms, the heteroaromatic moiety having at least one heteroatom selected from sulfur, oxygen and nitrogen.

“Aryl” refers to a hydrocarbon ring system radical comprising hydrogen, 6 to 18 carbon atoms and at least one aromatic ring. In an aspect the aryl ring system has 6 to 12 carbon atoms. In an aspect the aryl ring system has 6 to 10 carbon atoms. For purposes of this disclosure, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted by one or more substituents independently selected at each occurrence.

“Heteroaryl” refers to “aryl” as defined herein, wherein the aromatic ring includes one or more heteroatoms, preferably selected from N, O and S. Thus, a heteroaryl radical refers to an aromatic ring system radical wherein the ring atoms are selected from carbon, nitrogen, oxygen and sulfur, and include at least one of nitrogen, oxygen and sulfur. For purposes of this disclosure, the heteroaryl radical may be a monocyclic, bicyclic, tricyclic or tetracyclic ring system, which may include fused or bridged ring systems. Optionally, the heteroaryl radical is a 5-, 6- or 7-membered heteroaryl group. When there are multiple 0 and S atoms in the heteroaryl ring system, the 0 atoms and/or S atoms are preferably not linked directly to one another. Exemplary heteroaryl groups include 5-membered rings, such as pyrrole, pyrazole, imidazole, 1,2,3-triazole, 1,2,4-triazole, tetrazole, furan, thiophene, selenophene, oxazole, isoxazole, 1,2-thiazole, 1,3-thiazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole. The heteroaryl group may be a 6-membered ring, such as pyridine, pyridazine, pyrimidine, pyrazine, 1,3,5-triazine, 1,2,4-triazine, 1,2,3-triazine, 1,2,4,5-tetrazine, 1,2,3,4-tetrazine, 1,2,3,5-tetrazine, or fused rings including a 6-membered ring such as indole, isoindole, indolizine, indazole, benzimidazole, benzotriazole, purine, naphthimidazole, phenanthrimidazole, pyridimidazole, pyrazinimidazole, quinoxalinimidazole, benzoxazole, naphthoxazole, anthroxazole, phenanthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquin-oline, acridine, phenothiazine, phenoxazine, benzopyridazine, benzopyrimi-dine, quinoxaline, phenazine, naphthyridine, azacarbazole, benzocarboline, phenanthridine, phenanthroline, thieno[2,3b]thiophene, thieno[3,2b]thiophene, dithienothiophene, isobenzothiophene, dibenzothiophene, and benzothiadiazo-thiophene. Unless stated otherwise specifically in the specification, the ring atoms of a heteroaryl group may be optionally substituted by one or more substituents independently selected at each ring atom.

A substituted C₁-C₂₀ aliphatic or aromatic moiety refers to a C₁-C₂₀ aliphatic or aromatic moiety having one or more substituents, where a “substituent” refers to monovalent group that may be attached to a mentioned moiety. For example, a “substituted phenyl” refers to a phenyl ring having 1, 2, 3 or 4 substituents attached to the phenyl ring. Substituents may be selected from halogen, C₁-C₆alkyl, C₁-C₆haloalkyl, C₁-C₆hydroxyalkyl, —OH, —O(C₁-C₆alkyl), —O(C₁-C₆haloalkyl), —O(C₁-C₆hydroxyalkyl), —S(C₁-C₆alkyl), —S(C₁-C₆haloalkyl), —S(C₁-C₆hydroxyalkyl), cyano, amino (—NH₂), formyl (—CHO), carboxylic acid (—COOH), carboxylate ester (—COOR where R is a C₁-C₁₀ alkyl group).

These thiol compounds include alkyl thiols which may be linear, branched or cyclic, aryl thiols, charged thiol compounds, polymers that contain a free thiol, peptides that contain a free thiol, heterocycles that contain a free thiol, drugs or biologically active compounds with a free thiol, growth factors with a free thiol, antibodies or antibody fragments with a free thiol and proteins with a free thiol. Examples of such thiol compounds include, and are not limited to thiophenol, 2-phenylethanethiol, triphenylmethanethiol, 4-methylbenzenethiol, 4-aminothiophenol, 2-aminothiophenol, 4-methoxy-α-toluenethiol, 4-nitrothiophenol, 4-tert-butylbenzenethiol, 2-mercapto-2-phenylacetic acid, 4-mercaptobenzoic acid, 2-mercaptobenzoic acid (thiosalicylic acid), 3-mercapto-1-propanol, 1-mercapto-2-propanol, 4-mercapto-1-butanol, 3-mercapto-1-hexanol, 6-mercapto-1-hexanol, 8-mercapto-1-octanol, 9-mercapto-1-nonanol, 11-mercapto-1-undecanol, 4-mercapto-4-methylpentan-2-ol, ethanethiol, 1-propanethiol, 2-propanethiol, 1-butanetiol, 1-Pentanethiol, 1-hexanethiol, 2-ethylhexanethiol, 1-heptanethiol, 1-octanethiol, 1-nonanethiol, 1-decanethiol, 1-undecanethiol, 1-dodecanethiol, 1-tetradecanethiol, 1-hexadecanethiol, cis-9-octadecene-1-thiol, 1-octadecanethiol, 2-methyl-1-butanethiol, 3-methyl-1-butanethiol, cycloalkyl, cyclohexanethiol, cyclopentanethiol, sodium 3-mercapto-1-propanesulfonate, sodium mercaptopyruvate, 6-mercaptohexanoic acid, 8-mercaptooctanoic acid, 11-mercaptoundecanoic acid, 16-mercaptohexadecanoic acid, sodium 2-mercaptoethanesulfonate, 3-mercaptopropionic acid, 2-amino-4-mercaptobutyric acid (DL-homocysteine), L-cysteine, 11-mercaptoundecylphosphoric acid, 2-mercapto-1-methylimidazole, 1-benzyl-2-mercaptoimidazole, 2-mercapto-6-methylpyridine, 3-mercapto-2-butanone, 3-mercapto-3-methyl-1-butyl-1-formate, 3-mercapto-3-methylbutan-1-ol, 7-mercapto-4-methylcoumarin, 2-mercapto-4-methyl-5-thiazoleacetic acid, 2-mercapto-5-nitrobenzimidazole, 2-mercapto-5-benzimidazolesulfonic acid sodium salt dihydrate, 3-mercapto-N-nonylpropionamide, 2-mercapto-4-methylpyrimidine hydrochloride, 2-mercapto-2-phenylacetic acid, 2-mercapto-3-(trifluoromethyl)pyridine, 2-mercapto-N-m-tolylacetamide, and 4-mercapto-4-methylpentan-2-ol are exemplary thiol compounds.

Polymers with free thiols include but are not limited to Thiol-PEG3-phosphonic acid, poly(L-lactide), thiol terminated 5000, poly(L-lactide), thiol terminated 2500, PEG-SH 3000, PEG-SH 5000, thiol-functionalized hyaluronic acid, thiol-functionalized chitosan, thiol functionalized alginate, thiol functionalized dextran, thiol functionalized chondroitin sulfate and thiol functionalized carboxymethyl cellulose.

Examples of thiol functionalized hyaluronic acid include but are not limited to a thiol group linked to hyaluronic acid through a hydrazide compound as described in U.S. Pat. No. 7,981,871, through carbodiimide groups as described in U.S. Pat. No. 6,884,788, as well as those described in U.S. Pat. No. 8,124,757.

Examples of thiol functionalized chitosan include but are not limited to chitosan-cysteine conjugates, chitosan-thioglycolic acid conjugates and chitosan-4-thio-butylamidine conjugates.

Non-degradable thiol functionalized polymers include but are not limited to polycarbophil-cysteamine conjugates, polycarbophil-cysteine conjugates, and poly(acrylic acid)-homocysteine conjugates.

Thiolated peptides or peptides that contain at least of free thiol, include but are not limited to a cysteine terminated peptide containing residues 73-92 of the knuckle epitope of BMP-2 (N→C: KIPKASSVPTELSAISTLYLSGGC), thiolated gelatin (see, e.g., U.S. Pat. Nos. 7,928,069 and 7,981,871), cysteine terminated cell adhesion epitopes such as Arg-Gly-Asp (RGD), Arg-Gly-Asp-Ser (RGDS) and Ile-Lys-Val-Ala-Val (IKVAV), cysteine terminated TAT peptide (GRKKRRQRRRPQ), laminin peptide sequence Cys-Ser-Arg-Ala-Arg-Lys-Gln-Ala-Ala-Ser-Ile-Lys-Val-Ala-Val-Ser-Ala-Asp-Arg (CSRARKQAASIKVAVSADR; lam-IKVAV), and cysteine terminated Elastin-like polypeptides such as those of the sequence (V P G X G)n where X=any amino acid except proline.

Thiol containing drugs include but are not limited to Captopril, Thiorphan, Tiopronin and Penicillamine.

Suitable proteins that contain a cysteine group include but are not limited to an IL-3 variant (see, e.g., U.S. Pat. No. 5,166,322), an IL-2 variant (see, e.g., U.S. Pat. No. 5,206,344), protease nexin-1 varients (see, e.g., U.S. Pat. No. 5,766,897), Cysteine variants of granulocyte-macrophage colony-stimulating factor (see, e.g., U.S. Pat. No. 7,148,333; and Bioconjugate Chem., 2005, 16 (5), pp 1291-1298; DOI: 10.1021/bc050172r), cysteine modified maize ribosome-inactivating protein (maize RIP) [see, e.g., Toxins 2016, 8, 298; doi:10.3390/toxins8100298], cysteine analog of erythropoietin [see, e.g., Int J Nanomedicine. 2011; 6: 1217-1227; doi: 10.2147/IJN.S19081], reduced antibody fragments [see, e.g., Protein Eng Des Sel (2007) 20 (5): 227-234.DOI: https://doi.org/10.1093/protein/gzm015], and cysteine analogues of Bone Morphogenetic Protein-2 (see, e.g., Bioconjugate Chem., 2010, 21 (10), pp 1762-1772; DOI: 10.1021/bc9005706.

Suitable growth factors that comprise a free thiol group include but are not limited to Cysteine Analogs of Human Basic Fibroblast Growth Factor (hbFGF) [see, e.g., Tropical Journal of Pharmaceutical Research October 2014; 13 (10): 1601-1607; http://dx.doi.org/10.4314/tjpr.v13i10.5; and Protein Expr. Purif. 2006 July;48(1):24-7https://doi.org/10.1016/j.pep.2006.02.002]).

In an aspect, the present disclosure provides a process comprising: reacting hydroxyl groups of hyaluronic acid (HA), or salts thereof, with divinyl sulfone (DVS) to provide a first derivative of the polymer; and reacting the first derivative of the hyaluronic acid, or salts thereof, with a nucleophile of a formula X′—Ar—Y to provide a second derivative of the polymer. The first derivative will have a number of ethenyl (vinyl) groups attached to sulfone groups that are, in turn attached through an oxyethylene group to the polymer. Some or all of these vinyl groups are reacted with a nucleophilic compound, e.g., a thiol derivative as described above. The extent to which these vinyl groups undergo reaction may be specified according to the present disclosure. In an aspect, all or nearly all, e.g., 100%, or 99-100%, or 98-100%, or 97-100%, or 96-100%, or 95-100% are substituted with the thiol derivative. In an aspect, partial substitution is achieved with the thiol derivative, e.g., 1-95% of the free available vinyl sulfone groups are derivatized.

For example, in an aspect the number of vinyl sulfone residues, that are attached to the hyaluronic acid, and that can be reacted with a free thiol-containing compound can be altered. The percentage of the residual vinyl sulfone groups reacted with a free thiol-containing compound can vary from 1% to 100%. NMR, such as ¹H-NMR, can be used to determine the percent substitution. When 100% substitution of the vinyl sulfone groups occurs, essentially all of the available vinyl sulfone residues attached to the polysaccharide have reacted with the free thiol-containing compound to form a thioether linkage. If less than 100% of the available vinyl sulfone groups react with the free thiol-containing compound, the hyaluronic acid will comprise both vinyl sulfone groups as well as compounds attached via a thioether linkage. The fraction of the repeat units of the polysaccharide that are substituted through a thioether linkage can be determined by NMR, usually ¹H-NMR. The percent substitution, often calculated on a molar basis, can range from 1% to 100%, preferably greater than 10% and more preferably greater than 25%.

In an aspect, the Michael addition reaction of a free-thiol compound with the vinyl sulfone residue on the hyaluronic acid can occur using a single free-thiol containing compound. In an aspect, the addition reaction can occur using more than 1 free thiol-containing compound in which the free thiol-containing compounds are different from each other.

FIG. 2 illustrate hyaluronic acid derivatization reactions according to the present disclosure. In FIG. 2, “A” represents hyaluronic acid or a salt thereof.

In FIG. 2, “B” identifies the product of reacting polymer A with divinyl sulfone (DVS) under basic conditions (NaOH in an aqueous solvent) Polymer B is a compound of the present disclosure. Polymer B is shown as two A polymers joined together through X linkages, where X represents a diethyl sulfone group of the formula —CH₂—CH₂—SO₂—CH₂—CH₂— which is linked at each of its ends to an oxygen atom that was formerly part of a hydroxyl group from polymer A. The X groups are created by reaction of two hydroxyl groups reacting with two vinyl groups of divinyl sulfone. The reaction takes place at pH greater than 12.5 and for a time period long enough that a crosslinked gel is formed. The X groups are shown linking together two different A polymers, however an X group may also link together two hydroxyl groups of a single A polymer to provide a polymer B according to the present disclosure. While only two A polymers are shown, the reaction can continue until multiple A Polymers are bound together to form a crosslinked gel.

In FIG. 2, “B” contains three X linkages between two A polymers in addition to three VS groups. A VS group is the result of a divinyl sulfone substitution reaction with a hydroxyl group of an A polymer. In order to create a VS group, one and only one of the two vinyl groups of a divinyl sulfone molecule reacts with one and only one hydroxyl group of a polymer A.

In an aspect, the polymer B still contains unreacted hydroxyl groups. For example, when a flask is charged with a desired amount of polymer A comprising a specified number of hydroxyl groups, the addition of DVS will consume at least 5%, or least 10%, or at least 20%, or at least 30%, or at least 40%, or at least 50%, or at least 60%, or at least 70%, or at least 80% of those initial hydroxyl groups in the formation of X and VS groups present in polymer B. The number of hydroxyl groups present after reaction of DVS may also, or alternatively be described in terms of the residual hydroxyl groups, so that at least 15%, or at least 20%, or at least 25%, or at least 30%, or at least 35%, or at least 40%, or at least 50%, or at least 60%, or at least 70%, or at least 80% of the initial hydroxyl groups are still present in polymer B. The number of hydroxyl groups present in polymer B may also be expressed as a range of the initial number of hydroxyl group present in polymer A, e.g., the conversion of polymer A to polymer B may consume 5-10% of the available hydroxyl groups, or in other aspects, 5-15%, or 5-20%, or 5-25%, or 5-30%, or 5-35%, or 10-15%, or 10-20%, or 10-25%, or 10-30%, or 10-35%, or 10-40% of the initially available hydroxyl groups.

In an aspect, the polymer B contains both X and VS substituents. In an aspect, the polymer B contains both X and VS substituents in a molar ratio of where the number of VS groups exceeds the number of X groups. However, in an aspect, the number of X groups exceeds the number of VS groups. In other aspects, the X groups provide at least 5%, 10%, 20%, 30%, 40%, 50%, 60%, 70%, 80%, or at least 90% of the total number of X and VS groups.

As shown in FIG. 2, polymer B may serve as a reactant to create either polymer C or polymer D, each of which is a polymer according to the present disclosure. To create polymer C, a mixture of nucleophilic compounds, represented as R₁SH and R₂SH in FIG. 2, is reacted with polymer B. In an aspect, the reaction of polymer B with the nucleophilic compounds occurs under basic conditions such that the nucleophilic compound acts as a nucleophile. The reaction is a Michael addition reaction. To create polymer D, a single nucleophilic compound, represented as R₁SH in FIG. 2, is reacted with polymer B. In an aspect, the reaction of polymer B with the nucleophilic compounds occurs under basic conditions such that the nucleophilic compound acts as a nucleophile. In an aspect, the reaction is a Michael addition reaction. The present disclosure provides polymer B, polymer C, polymer D as well as reactions to create polymer B from polymer A, reactions to create polymer C from polymer B, and reactions to create polymer D from polymer B. In an aspect, each of polymers A, B and C is a derivatized hyaluronic acid.

Polymer D contains X moieties which link together two polymer A chains. In addition, polymer D contains Z—S—R₁ moieties which are created by the reaction of the vinyl sulfone (VS) groups of polymer B with thiol compound R₁SH to provide —O—CH₂—CH₂—SO₂—CH₂—CH₂—S—R₁ moieties, which are abbreviated as Z—S—R1 moieties in FIG. 2. In an aspect, the present disclosure provides polymer D having a mixture of X groups and Z—S—R₁ groups. In an aspect, X groups provide at least 10%, or at least 20%, or at least 30%, or at least 40%, or at least 50%, or at least 60%, or at least 70%, or at least 80%, or at least 90% of the total of the X and Z—S—R₁ groups. In an aspect, Z—S—R₁ groups provide at least 10%, or at least 20%, or at least 30%, or at least 40%, or at least 50%, or at least 60%, or at least 70%, or at least 80%, or at least 90% of the total of the X and Z—S—R₁ groups.

In an aspect, the present disclosure provides polymer E having a structure as set forth in FIG. 2. In an aspect, the present disclosure provides polymer F having a structure as identified in FIG. 2. In an aspect, the present disclosure provides polymer G having a structure as identified in FIG. 2. In yet an aspect, the present disclosure provides polymer H having a structure as identified in FIG. 2.

As shown in FIG. 2, polymer A may be reacted with divinyl sulfone under basic conditions to provide polymer E. In an aspect, the reaction occurs at a pH >12, in the presence of excess divinyl sulfone and for a time period that is short enough such that none to minimal crosslinking occurs. In an aspect, the reaction time is less than 30 minutes. As shown in FIG. 2, polymer E may be formed from polymer A by reaction of the hydroxyl groups of polymer A with divinyl sulfone (DVS) to convert them to vinyl sulfone (VS) groups. In polymer E, there are few, if any, X groups which link together two hydroxyl groups of polymer A. In various aspects, the VS groups constitute at least 80%, or at least 85%, or at least 90%, or at least, 95%, or at least 96%, or at least 97%, or at least 98%, or at least 99%, or at least 99.5%, or at least 99.9% of the total of X and VS groups present in polymer E.

Polymer E may be reacted with R₁SH, optionally in combination with one or more additional nucleophilic compounds, e.g., R₂SH, to provide polymers of structure F, G or H, as shown in FIG. 2. In an aspect, the reaction of polymer E with the nucleophilic compounds to provide polymers of structure F, G or H, occurs under basic conditions such that the nucleophilic compound acts as a nucleophile. In an aspect, the reaction is a Michael addition reaction. Polymer F has a mixture of residual VS groups and Z—S—R1 groups formed by reaction of VS groups with R₁SH. In an aspect, the charge of R₁SH is less than 100% of the total number of VS groups present on polymer E, calculated on a molar basis. Based on this stoichiometry, not all of the VS groups will react with R₁SH molecules, and accordingly polymer F has a mixture of VS and Z—S—R₁ groups. In an aspect, the reaction time is controlled and limited such that there is not enough time for complete reaction of all the R₁SH with available VS groups. In an aspect, VS groups provide at least 10%, or at least 20%, or at least 30%, or at least 40%, or at least 50%, or at least 60%, or at least 70%, or at least 80%, or at least 90% of the total of the VS and Z—S—R1 groups. In an aspect, Z—S—R₁ groups provide at least 10%, or at least 20%, or at least 30%, or at least 40%, or at least 50%, or at least 60%, or at least 70%, or at least 80%, or at least 90% of the total of the VS and Z—S—R1 groups.

Polymer G has a majority of Z—S—R₁ groups, and little or no X and VS groups. In various aspects, the Z—S—R₁ groups constitute at least 80%, or at least 85%, or at least 90%, or at least, 95%, or at least 96%, or at least 97%, or at least 98%, or at least 99%, or at least 99.5%, or at least 99.9% of the total of X, VS and Z—S—R₁ groups present in polymer G. In an aspect, Polymer G, the residual VS groups constitute less than 1% of the total X, VS and Z—S—R1 groups present in polymer G. Polymer G may be formed by reaction of polymer E and an equimolar or molar excess of R₁SH molecules, based on the moles of available VS groups.

Polymer H has a majority of Z—S—R₁ and Z—S—R₂ groups, and little or no X and VS groups. In various aspects, the total of the Z—S—R₁ and Z—S—R₂ groups constitute at least 80%, or at least 85%, or at least 90%, or at least, 95%, or at least 96%, or at least 97%, or at least 98%, or at least 99%, or at least 99.5%, or at least 99.9% of the total of X, VS, Z—S—R₁ and Z—S—R₂ groups present in polymer H. Polymer H may be formed by reaction of polymer E and a mixture of nucleophilic compounds, e.g., a mixture of R₁SH and R₂SH, such as shown in FIG. 2.

In an aspect, the present disclosure provides polymer I having a structure as set forth in FIG. 2. In an aspect, the present disclosure provides polymer J which is a crosslinked gel prepared as shown in FIG. 2. In an aspect, the present disclosure provides polymer K which is a crosslinked gel prepared as shown in FIG. 2.

Polymer I (FIG. 2) has a mixture of Z—S—R₁ and VS substituents. In an aspect, the present disclosure provides polymer I having a mixture of VS groups and Z—S—R₁ groups. In an aspect, VS groups provide at least 10%, or at least 20%, or at least 30%, or at least 40%, or at least 50%, or at least 60%, or at least 70%, or at least 80%, or at least 90% of the total of the VS and Z—S—R₁ groups. In an aspect, Z—S—R₁ groups provide at least 10%, or at least 20%, or at least 30%, or at least 40%, or at least 50%, or at least 60%, or at least 70%, or at least 80%, or at least 90% of the total of the VS and Z—S—R₁ groups. Polymer I may be formed by reacting polymer G with divinyl sulfone under basic conditions. This reaction converts one or more of the hydroxyl groups present on polymer G (not shown in FIG. 2) to vinyl sulfone (VS) groups.

Polymers J and K are crosslinked gels which may be prepared as shown in FIG. 2. Polymer J may be formed by crosslinking polymer G. Polymer K may be formed by crosslinking polymer H. Crosslinkers that can be used to crosslink polymer G and H are described elsewhere herein.

In an aspect, the present disclosure provides polymer L having a structure as set forth in FIG. 2. In an aspect, the present disclosure provides polymer M having a structure as identified in FIG. 2. In yet an aspect, the present disclosure provides polymer N having a structure as identified in FIG. 2.

Polymer L as shown in FIG. 2 contains a mixture of Z—S—R₁ and Z—S—R₂ substituents. Polymer L may additionally contain hydroxyl substituents (not shown). In various aspects, the total of the Z—S—R₁ and Z—S—R₂ groups constitute at least 80%, or at least 85%, or at least 90%, or at least, 95%, or at least 96%, or at least 97%, or at least 98%, or at least 99%, or at least 99.5%, or at least 99.9% of the total of X, VS, Z—S—R₁ and Z—S—R₂ groups present in polymer L. Polymer L may be formed by reaction of polymer F, which contains Z—S—R₁ and VS substituents, with R₂SH, to thereby convert the VS substituents to Z—S—R₂ substituents via a Michael addition reaction. In an aspect this reaction occurs under basic reaction conditions.

Polymer M as shown in FIG. 2 contains a mixture of X, Z—S—R₁ and Z—S—R₂ groups. Polymer M may additionally contain hydroxyl substituents (not shown). Polymer M may be formed by adding a crosslinker, such as divinyl sulfone or other crosslinkers as described elsewhere herein, to Polymer L that contains residual hydroxyl groups. The crosslinker creates X groups between hydroxyl groups present on polymer L. In an aspect the crosslinking reaction occurs under basic conditions.

Polymer N as shown in FIG. 2 contains a mixture of Z—S—R₁ and —R— groups, where an R group forms a linkage between different polymer A chains. The R groups may be introduced by reacting a precursor polymer, such as polymer F or other polymer containing VS groups, with a polyfunctional nucleophile, such as R(SH)n where n is greater than or equal to 2. In R(SH)n, R represents an aliphatic or aromatic group that is optionally substituted. In an aspect, the reaction is a Michael addition reaction that occurs under basic conditions.

In an aspect, the present disclosure provides Gel O which is a crosslinked gel that may be formed as shown in FIG. 2. In an aspect, the present disclosure provides Gel P which is a crosslinked gel that may be formed as shown in FIG. 2.

Gel O may be formed from Polymer I by a two-step reaction. In a first step, polymer I is reacted with a nucleophilic compound, such as R₁SH, to convert VS groups present on polymer I, into the corresponding Z—S—R₁ groups to produce polymer Q1. In a second step, a crosslinker X is added to polymer Q1 to provide a crosslinked gel, O.

Gel P may be formed from Polymer I by a two-step reaction. In a first step, polymer I is reacted with a nucleophilic compound, such as R₂SH, to convert VS groups present on polymer I, into the corresponding Z—S—R₂ groups to produce polymer Q2. In a second step, a crosslinker X is added to polymer Q2 to provide a crosslinked gel, P.

Polymer I may also serve as a precursor to a crosslinked polymer having —R— groups as the linkage between polymer chains, as shown by Q in FIG. 2. The R groups may be introduced by reacting a polymer I, or another polymer containing VS groups, with a polyfunctional nucleophile, such as R(SH)n where n is greater than or equal to 2. In R(SH)n, R represents an aliphatic or aromatic group that is optionally substituted.

FIG. 3 illustrates polymer derivatization reactions according to the present disclosure. In FIG. 3, “A” represents hyaluronic acid or a salt thereof, which is likewise shown as polymer A in FIG. 2. However, in contrast to FIG. 2, the reaction schemes of FIG. 3 begin by performing a crosslinking reaction on polymer A.

As shown in FIG. 3, polymer A may be reacted with a crosslinking agent, to provide a crosslinked version of polymer A, which is denoted as polymer B in FIG. 3. Suitable crosslinking reactions for hyaluronic acid or salts thereof are described elsewhere herein. In an aspect, the crosslinking reaction may occur under basic reaction conditions.

In an aspect, the present disclosure provides polymer C having a structure as set forth in FIG. 3. In an aspect, the present disclosure provides polymer D having a structure as identified in FIG. 3. In yet an aspect, the present disclosure provides polymer E having a structure as identified in FIG. 3.

Polymer C in FIG. 3 may be formed by reacting polymer B with divinyl sulfone (DVS) under basic conditions. Under these reaction conditions, hydroxyl groups present on polymer B (not shown) react with DVS to convert hydroxyl groups to VS groups. In an aspect, VS groups provide at least 10%, or at least 20%, or at least 30%, or at least 40%, or at least 50%, or at least 60%, or at least 70%, or at least 80%, or at least 90% of the total of the VS and X groups present in polymer C. In an aspect, X groups provide at least 10%, or at least 20%, or at least 30%, or at least 40%, or at least 50%, or at least 60%, or at least 70%, or at least 80%, or at least 90% of the total of the VS and X groups present in polymer C.

Polymer D in FIG. 3 is a crosslinked polymer having both Z—S—R₁ and Z—S—R₂ substituents. Polymer D may be formed by reacting polymer C with a mixture of nucleophilic compounds, such as R₁SH and R₂SH as shown in FIG. 3. In an aspect, the reaction of polymer C with the nucleophilic compounds occurs under basic conditions such that the nucleophilic compound acts as a nucleophile. In an aspect, the reaction is a Michael addition reaction. In an aspect, the total of the Z—S—R₁ and Z—S—R₂ groups provide at least 10%, or at least 20%, or at least 30%, or at least 40%, or at least 50%, or at least 60%, or at least 70%, or at least 80%, or at least 90% of the total of the Z—S—R₁, Z—S—R₂ and X groups present in polymer D. In an aspect, X groups provide at least 10%, or at least 20%, or at least 30%, or at least 40%, or at least 50%, or at least 60%, or at least 70%, or at least 80%, or at least 90% of the total of the Z—S—R₁, Z—S—R₂ and X groups present in polymer D.

Polymer E in FIG. 3 is a crosslinked polymer having Z—S—R₁ substituents (but not having any and Z—S—R₂ substituents). Polymer E may be formed by reacting polymer C of FIG. 3 with a nucleophilic compound, such as R₁SH as shown in FIG. 3. In an aspect, the reaction of polymer C with the nucleophilic compound occurs under basic conditions such that the nucleophilic compound acts as a nucleophile. In an aspect, the reaction is a Michael addition reaction. In an aspect, the Z—S—R₁ groups provide at least 10%, or at least 20%, or at least 30%, or at least 40%, or at least 50%, or at least 60%, or at least 70%, or at least 80%, or at least 90% of the total of the Z—S—R₁ and X groups present in polymer E. In an aspect, X groups provide at least 10%, or at least 20%, or at least 30%, or at least 40%, or at least 50%, or at least 60%, or at least 70%, or at least 80%, or at least 90% of the total of the Z—S—R₁ and X groups present in polymer E.

As shown in FIG. 3, the present disclosure provides polymers of structure F and of structure G, as well as crosslinked gels thereof. Polymer F of FIG. 3 contains Z—S—R1 substituents, while polymer G contains a mixture of Z—S—R1 and Z—S—R2 substituents. Neither of polymers F or G are crosslinked polymers. However, each of polymers F and G may be treated with a crosslinking agent, or exposing to crosslinking conditions, to provide the corresponding crosslinked polymer which will have the form of a gel (identified as Gel H in FIG. 3). In an aspect, two or more of the polymers or gels shown in FIG. 2 and FIG. 3 can be mixed together in the presence of a crosslinker such that a composite crosslinked gel is obtained. Crosslinkers that can be used to crosslink these combinations of polymers or gels are described elsewhere herein.

In an aspect, the crosslinking reaction may occur under basic reaction conditions.

Thus, in an aspect, the present disclosure provides vinyl sulfone functionalization of a represents hyaluronic acid or a salt thereof followed by reaction of the vinyl sulfone substituent with one or more free thiol-containing compounds which is in turn followed by a second functionalization reaction with divinyl sulfone to produce a represents hyaluronic acid or a salt thereof that is functionalized with compounds through a thioether linkage as well as with vinyl sulfone functional groups. In an aspect, the above compounds can be further reacted with free thiol-contain compound. The molar ratio of the free thiol-compound used for the reaction can be altered such that 1% to 100% of the second added vinyl sulfone functional groups are reacted. The free thiol-containing compound that is used in the second Michael addition reaction can be the same or it can be different from that used in the first Michael addition reaction. For the second Michael addition reaction, a single free thiol-containing compound can be used or a mixture of 2 or more different free-thiol containing compounds can be used. In an aspect, at least one additional round of vinyl sulfone/free thiol-containing compound reactions cycles can be performed using the same free-thiol containing compound or one or more different free-thiol containing compounds.

A second derivative can be in a form that has minimal to no crosslinking as measured by the storage modulus (G′) and the loss modulus (G″). In an aspect, a second derivative has a storage modulus that is lower than the loss modulus at angular frequencies of less than about 0.6 Hz and a storage modulus (G′) that is greater than the loss modulus (G″) at angular frequencies greater than about 8 Hz for the oscillation frequency sweep analysis using a rheometer. In an aspect, the minimal to no crosslinking can be seen by the crossing of the storage modulus above the loss modulus in an oscillation frequency sweep.

In an aspect, the process of the present disclosure further comprises crosslinking the second derivative of the polymer, e.g., crosslinking by reacting the second derivative of the polymer with a crosslinking agent. Upon crosslinking, the second derivative is converted to a third derivative of the polymer, where the third derivative is a crosslinked polymer.

For example, in an aspect, hyaluronic acid, or a salt thereof, derivatized with one or more free-thiol containing compound and also comprises residual available vinyl sulfone functional groups can undergo crosslinking by subjecting a solution of the composition to basic conditions that are sufficient to allow the residual available vinyl sulfone group to react with a hydroxyl group of the polysaccharide. In an aspect, the reaction pH is greater than 11.2 and preferably in the 12.0 to 13 pH range. In an aspect the reaction pH is in a range of about 11.5 to about 13.4 pH. In an aspect the reaction pH is in a range from about 11.5 to about 12.4 pH. The amount of residual vinyl sulfone functional groups, often measured as percent substitution as measured by ¹H-NMR, reaction time and reaction temperature can be selected to achieve the desired degree of crosslinking.

In an aspect, hyaluronic acid, or a salt thereof, derivatized with one or more free-thiol containing compounds and also comprises residual available vinyl sulfone functional groups can be mixed with hyaluronic acid, or a salt thereof, derivatized with one or more free-thiol containing compounds and also comprises residual available vinyl sulfone functional groups wherein the free-thiol containing compounds can be the same or different or a combination thereof. The resultant mixture can undergo crosslinking by subjecting a solution of the composition to basic conditions that are sufficient to allow the residual available vinyl sulfone group to react with a hydroxyl group of the polysaccharide. In an aspect, the reaction pH is greater than 11.2 and preferably in the 12.0 to 13 pH range. In an aspect the reaction pH is in a range of about 11.5 to about 13.4 pH. In an aspect the reaction pH is in a range of about 11.5 to about 12.4 pH. The amount of residual vinyl sulfone functional groups, often measured as percent substitution as measured by 1H-NMR, reaction time and reaction temperature can be selected to achieve the desired degree of crosslinking.

In an aspect, a non-derivatized hyaluronic acid, or a salt thereof, can be added to the crosslinking reaction mixtures described above and the resultant mixture can undergo crosslinking by subjecting a solution of the composition to basic conditions that are sufficient to allow the residual available vinyl sulfone group to react with a hydroxyl group of the hyaluronic acid, or a salt thereof. In an aspect, the reaction pH is greater than 11.2 and preferably in the 12.0 to 13 pH range. In an aspect the reaction pH is in a range of about 11.5 to about 13.4 pH. In an aspect the reaction pH is in a range of about 11.5 to about 12.4 pH. The amount of residual vinyl sulfone functional groups, often measured as percent substitution as measured by 1H-NMR, reaction time and reaction temperature can be selected to achieve the desired degree of crosslinking.

As mentioned above, the crosslinking may be achieved by using an external crosslinking agent. In an aspect, a crosslinking agent is added to the second derivative of the polymer. Exemplary crosslinking agents that could be used include: carbodiimides, bisepoxides, divinyl sulfone derivatives, and combinations thereof. Another suitable crosslinking agent is a multiple thioether derivative. In an aspect, at least 2 (could be 2, 3, 4, etc.) different thioether derivatives are combined with a crosslinking agent and conditions are adjusted such than composition becomes either fully crosslinked or partially crosslinked. In this case, exemplary crosslinking agents include, without limitation, carbodiimides, bisepoxides, divinyl sulfone derivatives and combination thereof.

For example, in an aspect, a hyaluronic acid, or a salt thereof, derivatized with one or more free-thiol containing compounds can be crosslinked by adding a crosslinking agent and adjusting the pH of the reaction mixture such that the polysaccharide forms a crosslinked composition. Crosslinking agents that can be used include but are not limited to biscarbodiimides, bisepoxides, divinyl sulfone derivatives, di-isocyanates, dihalide chlorides, disuccinimidyl derivatives and combinations thereof.

Biscarbodiimide compounds can include but are not limited to para-phenylenebis-(ethyl)-carbodiimide, 1,6-hexamethylene bis(ethylcarbodiimide), 1,8-octamethylene bis(ethylcarbodiimide), 1,10 decamethylene bis(ethylcarbodiimide), 1,12 dodecamethylene bis(ethylcarbodiimide), PEG-bis(propyl(ethylcarbodiimide)), 2,2′-dithioethyl bis(ethylcarbodiimde), 1,1′-dithio-p-phenylene bis(ethylcarbodiimide); para-phenylene-bis(ethylcarbodiimide), and 1,1′-dithio-m-phenylene bis(ethylcarbodiimide).

When utilizing a biscarbodiimide crosslinker, the biscarbodiimide is mixed with a buffered aqueous solution of the derivatized carboxylic acid containing polysaccharide. The target pH of the buffered solution can be between pH 5 and pH 6.5.

Bisepoxide compounds can include but are not limited to 1,4-butanediol diglycidyl ether (BDDE), 1,2,7,8-diepoxyoctane (DEO), poly(ethylene glycol) diepoxide. When utilizing a bisepoxide crosslinker, the bisepoxide is mixed with an aqueous solution of the derivatized polysaccharide and the pH is raised to a pH >9. In an aspect, the reaction can be carried out at 40° C. for greater than 4 hours to produce a crosslinked composition. In an aspect, the reaction can be carried out at about 25° C. to about 50° C. for a duration of between about 30 minutes and about 4 hours to produce a crosslinked composition. In an aspect, the reaction can be carried out at about 45° C. to about 55° C. for a duration of between about 2 hours and about 4 hours to produce a crosslinked composition.

Divinyl sulfone crosslinkers can include but are not limited to divinyl sulfone and poly(ethylene glycol) bisvinyl sulfone.

When utilizing a divinyl sulfone crosslinker, the reaction pH in an aqueous solution can be raised to a pH greater than 12 to effect crosslinking. In an aspect, the reaction pH in an aqueous solution can be raised to a pH greater than 11.2 to effect crosslinking. The degree of crosslinking can be altered by changing the amount of crosslinking agent added, reaction time, the reaction pH and reaction temperature.

In an aspect, a mixture of at least 2 different thioether derivatized hyaluronic acid, or a salt thereof, can be mixed together, a crosslinking agent can be added and the reactions conditions adjusted such than compositions are crosslinked. The relative ratios of the different derivatized polysaccharides can be altered such that crosslinked compositions with different properties are obtained. These properties include but are not limited to equilibrium swelling, swelling rate, drug release characteristics, elastic modulus, storage modulus, loss modulus, degradation, tensile strength, injectability, tissue adhesiveness and lubricity.

In an aspect, at least 2 different crosslinking agents can be used to crosslink the derivatized hyaluronic acid, or a salt thereof. In an aspect, two different crosslinkers from the same group could be used to crosslink the composition. For example, divinyl sulfone and poly(ethylene glycol) bisvinyl sulfone or 1,4-butanediol diglycidyl ether (BDDE) and poly(ethylene glycol) diepoxide could be used.

In an aspect, two different crosslinkers from different groups could be used. For example, divinyl sulfone and 1,4-butanediol diglycidyl ether (BDDE) may be used to crosslink the derivatized hyaluronic acid, or a salt thereof. In an aspect, the crosslinker can be added sequentially such that initial crosslinking occurs in the presence of the first crosslinked and then the second crosslinker is added such that secondary crosslinking occurs. The reaction conditions may be changed after the first crosslinking reaction and prior to the second crosslinking reaction. Reaction conditions such as temperature, pH, buffer, ionic strength and solvent composition can be altered.

The derivative of this disclosure can be crosslinked to form a gel. A method used to crosslink the derivative can include but are not limited to varying the pH of the reaction, the concentration of the reagents, the duration of the crosslinking steps, the temperature at which the crosslinking occurs, the ionic strength of the solution, the order of addition of reagents, the mixing steps of the crosslinking system as well as combinations of these parameters.

In an aspect, the crosslinking of the gel can occur by dissolving the derivative in an aqueous solution of NaOH that has a pH in the range of 11.2 to 13.4, adding a crosslinker to the solution, mixing the solution for a period of time and then allowing the solution to crosslink. In an aspect, the derivative is dissolved in an aqueous solution, the pH of the solution is adjusted to 11.2 to 13.4, the crosslinker is added, the solution is mixed for a period of time and the solution is allowed to crosslink. In an aspect, the derivative is dissolved in an aqueous solution that comprises the crosslinking agent, the pH of the solution is adjusted to 11.2 to 13.4, the solution is mixed for a period of time and the solution is allowed to crosslink. In an aspect, the derivative is dissolved in an aqueous solution that has a pH of about 11.2 to about 13.4 and that further comprises the crosslinking agent, the solution is mixed for a period of time and the solution is allowed to crosslink.

In an aspect, the crosslinking time can be between about 30 minutes and about 24 hours. In an aspect, the crosslinking time can be between about 1 hour and about 8 hours. In an aspect, the crosslinking time can be between about 2 hours and about 6 hours. In an aspect, the crosslinking time can be between about 2 hours and about 4 hours.

The crosslinking reaction can be performed at less than 18° C., or above 18° C. In an aspect, the crosslinking reaction is performed at about 18° C. to about 30° C. In an aspect, the crosslinking reaction is performed at about 30° C. to about 40° C. In an aspect, the crosslinking reaction is performed at about 40° C. to about 60° C. In an aspect, the crosslinking reaction can occur at two different temperatures with the first reaction temperature being lower than the second reaction temperature. In an aspect, the first reaction temperature can be higher than the second reaction temperature.

In an aspect, the crosslinking of the gel can occur by dissolving the derivative in an aqueous solution of NaOH that has a pH in the range of 11.2 to 13.4, adding a crosslinker to the solution, mixing the solution for a period of time and then allowing the solution to crosslink at a temperature in the range of 20° C. to 30° C. for a period of 1 to 2 hours. The temperature of the reaction solution is then increased to about 45° C. to 55° C. and the crosslinking reaction is continued for an additional 2-5 hours.

In an aspect, the crosslinking of the gel can occur by preparing a pH 11.2 to 13.4 aqueous solution that comprises the derivative and the crosslinker dissolving the derivative in an aqueous solution of NaOH that has a pH in the range of 11.2 to 13.4, adding a crosslinker to the solution, mixing the solution for a period of time and then allowing the solution to crosslink at a temperature in the range of 35° C. to 60° C. for a period of 1 to 2 hours. An additional quantity of the derivative dissolved in an aqueous solution of NaOH that has a pH in the range of 11.2 to 13.4 is added to the initial solution and the crosslinking reaction is continued for an additional 1-6 hours. In an aspect, the concentration of the derivative in the second added solution is lower than that of the derivative in the first solution. In an aspect, the concentration of the derivative in the second added solution is the same as that of the derivative in the first solution. In an aspect, the concentration of the derivative in the second added solution is greater than that of the derivative in the first solution.

In an aspect, an aqueous solution at pH about 11.2 to 13.4 that comprises the derivative is prepared and allowed to crosslink for a period of time (e.g. 30 min to about 6 hours). A crosslinker solution is then added to the reaction mixture and the crosslinking reaction is allowed to continue for an additional period of time. In an aspect the first reaction time is between about 30 min and about 6 hours and the second reaction time is between about 30 min and about 8 hours. In an aspect, the pH of the crosslinking reaction can be changed during the reaction. In an aspect, the first reaction pH is lower than the second reaction pH. In an aspect, the first reaction pH is higher than the second reaction pH.

Additional derivative of the disclosure or additional hyaluronic acid can be added during the crosslinking reaction. In an aspect, the concentration of the added derivative of the disclosure or added hyaluronic acid, is lower than that of the starting concentration of the derivative of the disclosure. In an aspect, the concentration of the added derivative of the disclosure or added hyaluronic acid, is higher than that of the starting concentration of the derivative of the disclosure.

The crosslinked gel can be formed using derivatives of the disclosure that have different levels of substitution or different molecular weights. In an aspect, the level of substitution of the different derivatives of the disclosure that are used to prepare the crosslinked gels, differ by at least 10%. In an aspect, the level of substitution of the different derivatives of the disclosure that are used to prepare the crosslinked gels, differ by at least 20%. In an aspect, the level of substitution of the different derivatives of the disclosure that are used to prepare the crosslinked gels, differ by at least 30%.

In an aspect, the molecular weight of the different derivatives of the disclosure that are used to prepare the crosslinked gels, differ by at least 10%. In an aspect, the molecular weight of the different derivatives of the disclosure that are used to prepare the crosslinked gels, differ by at least 50%. In an aspect, the molecular weight of the different derivatives of the disclosure that are used to prepare the crosslinked gels, differ by at least 100%.

The derivative of the disclosure can be crosslinked without the use of an external crosslinking agent or without the use of residual vinyl sulfone groups. In an aspect, the derivative of the disclosure is crosslinked by dissolving the derivative in an aqueous solution, adjusting the pH of the solution to greater than about 11.4 and allowing the derivative to crosslink to form a gel. In an aspect, the derivative is dissolved in an aqueous solution that has a pH of greater than about 11.4 and the derivative is allowed to incubate in the solution until it is crosslinked. In an aspect the pH of the solution is about pH 11.4 to about pH 13.5. In an aspect the pH of the solution is about pH 11.4 to about pH 11.8. In an aspect the pH of the solution is about pH 11.8 to about pH 12.2. In an aspect the pH of the solution is about pH 12.2 to about pH 12.6. In an aspect the pH of the solution is about pH 12.6 to about pH 13.0. In an aspect the pH of the solution is about pH 13.0 to about pH 13.5. The crosslinking reaction can take place at room temperature. In an aspect, the crosslinking reaction can take place at about 20° C. to about 25° C. In an aspect, the crosslinking reaction can take place at about 25° C. to about 35° C. In an aspect, the crosslinking reaction can take place at about 35° C. to about 45° C. In an aspect, the crosslinking reaction can take place at about 45° C. to about 60° C. In an aspect, the solution to be crosslinked can further comprise a second polymer. In an aspect, the duration for the crosslinking reaction is between about 30 minutes and about 24 hours. In an aspect, the duration for the crosslinking reaction is between about 1 hour and about 8 hours. In an aspect, the duration for the crosslinking reaction is between about 2 hour and about 6 hours. In aspect, the second polymer can be an excipient polymer as described herein.

The derivative of the disclosure can be crosslinked using a crosslinker with the percent mass ratio of crosslinker to hyaluronic acid derivative being about 0.1% (w/w) to about 75% (w/w). In an aspect, the percent mass ratio crosslinker to hyaluronic acid derivative is about 0.1% (w/w) to about 1% (w/w). In an aspect, the percent mass ratio crosslinker to hyaluronic acid derivative is about 1% (w/w) to about 5% (w/w). In an aspect, the percent mass ratio crosslinker to hyaluronic acid derivative is about 5% (w/w) to about 10% (w/w). In an aspect, the percent mass ratio crosslinker to hyaluronic acid derivative is about 10% (w/w) to about 15% (w/w). In an aspect, the percent mass ratio crosslinker to hyaluronic acid derivative is about 15% (w/w) to about 20% (w/w). In an aspect, the percent mass ratio crosslinker to hyaluronic acid derivative is about 20% (w/w) to about 30% (w/w). In an aspect, the percent mass ratio crosslinker to hyaluronic acid derivative is about 30% (w/w) to about 50% (w/w). In an aspect, the percent mass ratio crosslinker to hyaluronic acid derivative is about 50% (w/w) to about 75% (w/w).

The derivative of the disclosure can be crosslinked using a crosslinker at a pH as described herein and in the presence of one or more agents that can increase the osmolality of the crosslinking solution. In an aspect, agent that can be used to increase the osmolality can be ionic or non-ionic. In an aspect, the agent that can be used to increase the osmolality is a sodium salt, a potassium salt, a calcium salt, a magnesium salt, a zinc salt, or a combination thereof. In an aspect, the agent that can be used to increase the osmolality is a saccharide. In an aspect, the saccharide is dextrose, sucrose, mannose, mannitol, sorbitol, glucose or a combination thereof. In an aspect, the agent that can be used to increase the osmolality can comprise about 0.01% (w/w) to about 10% (w/w) of the crosslinking solution. In an aspect, the agent that can be used to increase the osmolality can comprise about 0.01% (w/w) to about 0.1% (w/w) of the crosslinking solution. In an aspect, the agent that can be used to increase the osmolality can comprise about 0.1% (w/w) to about 1% (w/w) of the crosslinking solution. In an aspect, the agent that can be used to increase the osmolality can comprise about 1% (w/w) to about 5% (w/w) of the crosslinking solution. In an aspect, the agent that can be used to increase the osmolality can comprise about 5% (w/w) to about 10% (w/w) of the crosslinking solution.

The conditions used to crosslink the derivatives of the disclosure can be different at the start of the crosslinking reaction as compared to the conditions at the end of the reaction. In an aspect, the pH, temperature, reagent concentrations, crosslinker ratio, osmolality or a combination thereof can be adjusted during the course of the crosslinking reaction such that one or more of these parameters are different from the initial value as compared to the final value for the crosslinking reaction.

In an aspect, crosslinked compositions can be prepared though ionic crosslinking. This can be accomplished by mixing a composition of this disclosure that has a negative charge with a compound that has two or more positive charges. In an aspect, a solution of the composition of this disclosure that has a positive charge can be prepared and then mixed with a solution of a compound that has two or more positive charges. Inorganic compounds that can be used include but are not limited to calcium chloride, zinc chloride, ferric chloride, aluminum chloride, chromium sulfate, and aluminum sulfate. Polymeric compositions that can be used include polymers that comprise more than two lysine, arginine or histidine amino acids, chitosan and chitosan derivatives, deacetylated hyaluronic acid, polyethyleneimine (PEI), poly(N,N-dimethylaminoethylmethacrylate), poly(4-vinylpyridine), polyethyleneglycol-polylysine block copolymers (PEG-PLL), dextran grafted polylysine copolymers, or combinations thereof.

In an aspect, the positively charged or the negatively charged polymer can first be applied. This can then be followed by application of the oppositely charged polymer such that at the interface of the two layers, ionic interactions occur such than compositions are crosslinked together. In an aspect, the process can be repeated at least one more time.

The second derivative of the polymer may be crosslinked via internal and external crosslinking. For example, in an aspect, a hyaluronic acid, or a salt thereof, derivatized with one or more free-thiol containing compound and also comprising residual available vinyl sulfone functional groups can be crosslinked in the presence of an external crosslinking agent. In an aspect, the reaction conditions can be adjusted such than residual available vinyl sulfone groups and the added external crosslinked react simultaneously. For example, divinyl sulfone can be added as the external crosslinker and then the pH can be increased to a pH >12 which will result is crosslinking.

As another example, the crosslinking via the residual available vinyl sulfone functional groups can take place first which is then followed by the addition of the external crosslinker. The reaction conditions, for example pH, can be changed to affect the crosslinking reaction of the external added crosslinker. For example, the pH of the derivatized polysaccharide that contains the residual available vinyl sulfone functional groups can be raised to a pH >12. Once the reaction has been reached the desired level, the pH can be changed to between pH 5 and pH 6.5 with a buffer and then biscarbodiimide crosslinker, for example para-phenylenebis-(ethyl)-carbodiimide, can be added to the reaction mixture and allowed to react until the desired level of crosslinking is obtained. In an aspect, the biscarbodiimide crosslinking can take place first by adjusting the pH of the derivatized polysaccharide to between 5 and 6.5, adding the biscarbodiimide, allowing the crosslinking to proceed to the desired level, then raising the pH to pH >12 to allow the residual vinyl sulfone functional groups to crosslink.

In an aspect, a hyaluronic acid, or a salt thereof, derivatized with one or more free-thiol containing compound and also comprises residual available vinyl sulfone functional groups can be crosslinked in the presence of an external crosslinking agent that has at least two free thiol functional groups. These free thiol groups may be positioned upon a central molecule, “C”. The central molecule may be a linear or cyclic alkane, a polyethylene glycol (PEG) oligomer or polymer, or any other such suitable central molecule. In the case of PEG-based crosslinkers, the PEG may be linear, branched (having two polymer arms), or multi-armed (e.g., having 3, 4, 5, 6, 7, 8 or more polymer arms). Thus, in such instances, the central molecule will typically a linear PEG, a branched PEG having 2 arms, or a multi-armed PEG having PEG arms emanating from a central core.

Illustrative cores for such multi-armed polymers include erythritol, pentaerythritol, trimethylolpropane, glycerol, glycerol dimer (3,3′-oxydipropane-1,2-diol), glycerol oligomers, sorbitol, hexaglycerol, and the like.

Illustrative thiol crosslinkers include PEG-dithiol (HS-PEG-SH), 3-arm PEG-tri-thiol (glycerine core), 4-arm PEG-tetrathiol (pentaerythritol core), or 8-arm PEG-octa-thiol (hexaglycerine core). The foregoing multi-armed PEG reagents may also have fewer than all arms functionalized with thiol. Additional suitable thiol reagents having PEG as the central molecule are available from Laysan Bio (Arab, Ala.), as well as aromatic dithiols such as those available from NanoScience. Other suitable thiol crosslinkers include dimercaptosuccinic acid, 2,3-dimercapto-1-propanesulfonic acid, Trimethylolpropane tris(3-mercaptopropionate), dithiol functionalized pluronics F127, dithiol functionalized F68, dihydrolipoic acid, peptides or proteins containing at least 2 cysteine amino acids with free thiol groups, thiol functionalized dextran, and thiol-functionalized hyaluronic acid.

The crosslinked hyaluronic acid based polymers as described herein can have a storage modulus (G′) that is greater than the loss modulus (G″) over the range of angular frequencies of about 0.1 rad/sec to about 10 rad/sec in an oscillation frequency sweep measurement.

Polymers of the present disclosure, e.g., the first, second and third derivatives of a polymer such a hyaluronic acid, or a salt thereof, may be processed into numerous forms. For the non-crosslinked compositions, exemplary forms of the compositions can be as a solution, a gel, a suspension, an emulsion, a film, an electrospun matrix, a fiber, a lyophilized solid, a rod, a disc, a powder or in a particulate form. The particulate form can be prepared by milling (e.g., jet milling, roller milling, cryomilling, mechanical milling) fragmentation, precipitation or grinding. For the crosslinked compositions, the forms of the composition can be as a gel, a suspension, a film, an electrospun matrix, a fiber, a lyophilized solid, a rod, a disc, a powder or in a particulate form. The particulate form can be prepared by milling (jet milling, roller milling, cryomilling, mechanical milling) fragmentation, precipitation or grinding.

A solution of the composition can be prepared by dissolving the composition in an appropriate solvent or a combination of solvents. For example, water or a combination of water and water-miscible solvent can be used. Water-miscible solvents can include but are not limited to methanol, ethanol, isopropanol, dimethyl formamide (DMF) acetone, 1,4-dioxane, pyridine, dimethyl sulfoxide (DMSO), tetrahydrofuran (THF) and acetonitrile. The prepared solutions can be sterilized by filtering through a 0.2 m sterile filter. In an aspect, a solution can be prepared using one derivatized polysaccharide. The concentration of the prepared solutions can range from, e.g., 0.01% (w/v) to about 50% (w/v). In an aspect, the concentration is in the 0.1% (w/v) to 10% (w/v) range.

A film of non-crosslinked compositions of this disclosure can be prepared by preparing a solution of the composition. This solution can be then placed in a mold or drawn out on a surface, for example, using a gardner knife. The surface used can be glass, metal foil, stainless steel, Teflon, nylon, polyethylene, polypropylene, silicone or a release liner. The solvent can then be removed to form the film. The rate of solvent removal can be altered by using at least one of the following parameters: temperature, air or inert gas flow and pressure. To increase the rate of solvent evaporation, the temperature could be increased, the air or inert gas flow rate could be increased or the pressure could be decreased. A combination of these process could also be used. To slow the rate of solvent evaporation, the temperature could be decreased, the air or inert gas flow rate could be reduced or the pressure could be increased. A combination of these process could also be used. A film can comprise one of the compositions of this disclosure. The films can also comprise two or more different compositions of this disclosure. A composite film can be prepared by preparing a first film and then casting a second film on top of the first film. A composite film can be prepared by casting additional layers sequentially on top of the previous layer. The layers of the composite film can comprise the same composition if the disclosure, different compositions of this disclosure or a combination thereof.

Lyophilized forms of the non-crosslinked compositions of this disclosure can be prepared by making a solution of the composition, freezing the solution and then placing the frozen composition under a vacuum such than solvent is sublimed off to leave the composition in the solid form. A lyophilized form of the composition of this disclosure can comprise one of the compositions of this disclosure. In an aspect, the lyophilized form of the composition of this disclosure can comprise two or more different compositions of this disclosure. The form of the lyophilized composition is dependent on the form of the contained into which the solution was poured and frozen. The form can be a rectangle, square, disk, triangle, trapezoid, rod or any other form in which a mold can be made.

The compositions of this disclosure can be in the form of a powder or particulate. The powder or particulate may be obtained directly via precipitation. A powder or particulate form can also be obtained through a milling, grinding, spray drying or fragmentation process. Films, precipitated composition, dried composition, lyophilized composition or compositions dried in a form can be further process via a milling process (jet milling, roller milling, cryomilling, mechanical milling), a grinding or a fragmentation process. A combination of these processes can be used. Material with particle size in the range of 100 nm to 5 mm can be prepared. Specific size ranges of the powdered or particulate composition of this disclosure can be prepared by separating the composition according to size using sieves. The distribution of particle sizes can be broad with a standard deviation of the average size of greater than 40%. The distribution of particle sizes can be narrow with a standard deviation of the average size of less than 30%. The final powdered or particulate form of the compositions of this disclosure can comprise a single distribution of average particle sizes or it can comprise two or more distributions of particles prepared by mixing particles of different average particle size.

The compositions of this disclosure can be formed into a solid form by preparing a solution of the composition in a solvent that can be removed, pouring this solution into a mold of a specific shape and then removing the solvent such that a solid form of the composition is obtained. The molds used can be of various shapes and can include but are not limited to cubes, rectangles, rods, semi-circular rode and tubes. The solid composition of this disclosure can then be removed from the mold.

The composition of the disclosure can be processed into an electrospun matrix. In this process, a solution of the composition of the disclosure is prepared. The solvent used can be an organic solvent, water or a combination thereof. For example, for hyaluronic acid based compositions, water/ethanol or water/dimethylformamide (DMF) solvent mixtures can be used. In an aspect, dimethyl sulfoxide can be used as the solvent. In an aspect, dimethyl sulfoxide/water combinations can be used as the solvent. Solutions with a concentration of 0.5 to 5% (w/v) can be prepared. The solution that is to be electrospun can be placed in a syringe with a needle. The syringe is then placed in a syringe pump. The needle can have a blunt end and an inner diameter in the range of 0.25 to 1 mm. The needle and collection plate are attached to a high voltage supply. A voltage is then applied to the system. The applied voltage can be in the 10 kV to 45 kV. The syringe pump can extrude the solution. The flow rate of the syringe pump can be in the range of 10 μL/min to 1000 μL/min. The collector plate can be static, rotating or moving in a specific linear direction to give the fibers some directional orientation. The shape of the collector plate can be varied with the collector plate having but not limited to the following shapes: a flat surface, a textured surface, a curved surface, a square rod, a rectangular rod, a round mandrel, an oval mandrel, a semi-circular mandrel or a combination of these shapes. The temperature of the solution can be controlled as well as the collection plate and the surrounding environment. The distance of the needle tip to the collector plate can be altered. The distance of the needle tip to the collector plate can be in the 2-20 cm range. The collection plate can also be submerged in or sprayed with a solvent that assists in the precipitation of the newly spun fibers. For example, an ethanol bath may be used during the electrospinning of hyaluronic acid based compositions of this disclosure.

The composition of this disclosure can be processed into the form of a fiber. A solution of the composition of the disclosure is prepared. This solution is then extruded through an orifice to produce a solvent containing fiber. This fiber can be extruded into one or more solvent baths that assists in the formation of the fiber. The fiber is then dried to produce a solid fiber. The fibers can be prepared as a monofilament or a multifilament fiber. In an aspect, this fiber can then be further processed through an annealing step. U.S. Pat. Nos. 9,228,027, 5,520,916, 5,824,335, 8,389,498, US20130309494, US20150119783 describe exemplary methods to produce fibers from a polysaccharide. Each of these is incorporated by reference as means to produce fibers from compositions of this disclosure.

A fiber may be further processed by knitting or weaving. The knitted or woven composition can be in the form of a mesh. The mesh can comprise a single composition of this disclosure. In an aspect, the mesh can comprise 2 or more different compositions of this disclosure. In an aspect, the fiber can be further processed into a braid. The braid can comprise a single composition of this disclosure. In an aspect, the braid can comprise 2 or more different compositions of this disclosure. For meshes or braids that use different compositions of this disclosure, the compositions used can result in the mesh or braid having properties that change as a function of time. This includes degradation rates, water absorption, elongation, elastic modulus, tensile strength, physical shape, lubricity, cell adhesion, or a combination of these properties.

The knitted, woven or braided compositions can be manufacture in the presence of a degradable or non-degradable non-polysaccharide based composition. These compositions include polyethylene, polypropylene, polyethylene terephthalate (PET), polytetrafluorethylene (PTFE), nylon, polyurethane, polyester, polyanhydride, polyorthoester, polycarbonate, poly-ester-co-carbonate), polyhydroxybutyrates or combinations thereof.

Crosslinked polymers of the present disclosure may take various physical forms, including particle, film, lyophilized sponge, powder, particulate (e.g., milled, fragmented, precipitated and ground particulates), and may be formed in-situ, e.g., spray or liquid.

A film of crosslinked compositions of this disclosure can be prepared by preparing a solution of the composition to be crosslinked. The composition can be crosslinked by one of the methods described above. Prior to the final crosslinking process, the crosslinker is added, if required, and the solution pH can be adjusted to initiate the crosslinking process. This solution can be then placed in a mold or drawn out on a surface using a Gardner knife. The surface used can be glass, metal foil, stainless steel, Teflon, nylon, polyethylene, polypropylene or a release liner. The solution is then allowed to crosslink to form a gel composition. Heat can be applied to increase the rate of crosslinking. The solvent can then be removed to form the film.

The rate of solvent removal can be altered by using at least one of the following parameters: temperature, air or inert gas flow and pressure. To increase the rate of solvent evaporation, the temperature could be increased, the air or inert gas flow rate could be increased or the pressure could be decreased. A combination of these process could also be used. To slow the rate of solvent evaporation, the temperature could be decreased, the air or inert gas flow rate could be reduced or the pressure could be increased. A combination of these process could also be used. A film can comprise one of the compositions of this disclosure.

The films can also comprise two or more different compositions of this disclosure. A composite film can be prepared by preparing a first film and then casting a second film on top of the first film. A composite film can be prepared by casting additional layers sequentially on top of the previous layer. The layers of the composite film can comprise the same composition if the disclosure, different compositions of this disclosure or a combination thereof. The films can comprise both crosslinked and non-crosslinked compositions of this disclosure.

Lyophilized forms of the crosslinked compositions of this disclosure can be prepared by making a solution of the composition, crosslinking the composition, freezing the crosslinked composition and then placing the frozen composition under a vacuum such than solvent is sublimed off to leave the composition in the solid form. A lyophilized form of the composition of this disclosure can comprise one of the compositions of this disclosure. In an aspect, the lyophilized form of the composition of this disclosure can comprise two or more different compositions of this disclosure. The form of the lyophilized composition is dependent on the form of the contained into which the solution was poured and frozen. The form can be a rectangle, square, disk, triangle, trapezoid, rod or any other form in which a mold can be made. The lyophilized compositions of this disclosure can comprise both crosslinked and non-crosslinked compositions of this disclosure. In an aspect, the lyophilized composition can be rehydrated in the presence of a composition of this disclosure. In an aspect, a second lyophilization step may be performed on this rehydrated composition.

In an aspect, the solution used to rehydrate the first lyophilized composition, can be crosslinked. In an aspect, the composition produced from the second crosslinking step can be lyophilized to produce a dry porous composition.

The crosslinked compositions of this disclosure can be in the form of a powder or particulate. A powder or particulate form can also be obtained through a milling, grinding, spray drying or fragmentation process. Films, precipitated composition, dried composition, lyophilized compositions or compositions dried in a form can be further process via a milling process (jet milling, roller milling, cryomilling, mechanical milling), a grinding or a fragmentation process. A combination of these processes can be used. Material with particle size in the range of 100 nm to 5 mm can be prepared. Specific size ranges of the powdered or particulate composition of this disclosure can be prepared by separating the composition according to size using sieves. The distribution of particle sizes can be broad with a standard deviation of the average size of greater than 40%. The distribution of particle sizes can be narrow with a standard deviation of the average size of less than 30%. The final powdered or particulate form of the compositions of this disclosure can comprise a single distribution of average particle sizes or it can comprise two or more distributions of particles prepared by mixing particles of different average particle size.

The crosslinked compositions of the disclosure can be in the form of particles. In an aspect, the particles can further comprise an aqueous solution. In an aspect, the particles can further comprise an aqueous solution of hyaluronic acid or a salt thereof. In an aspect, the particles can further comprise an aqueous solution of the derivatized hyaluronic acid or a salt thereof of the compositions disclosed herein. In an aspect, the crosslinked composition can be passed through one or more meshes of a particular pore size or 2 or more meshes of different pore sizes. The particularized hydrogel can be screened through one or more meshes to isolate particles of a specific size range. In an aspect, the particles can have a volume average mean particle size of between 50 and 250 μm. In an aspect, the particles can have a volume average mean particle size of between 150 and 350 m. In an aspect, the particles can have a volume average mean particle size of between 200 and 500 μm. In an aspect, the particles can have a volume average mean particle size of between 500 and 750 μm. In an aspect, the particles can have a volume average mean particle size of between 600 and 1350 μm. In an aspect, particles of similar dimensions, can be formed by crosslinking a solution of the polymer of the disclosure that is dispersed in a non-solvent for the polymer of the disclosure such that particles that are obtained are about spherical in shape. Specific size ranges of the about spherical particles can be isolated by passing a solution of the particles through one or more mesh screens.

The crosslinked compositions of this disclosure can be formed into a solid form by preparing a solution of the composition in a solvent that can be removed, pouring this solution into a mold of a specific shape, crosslinking the composition in the mold, and then removing the solvent such that a solid form of the crosslinked composition is obtained. The molds used can be of various shapes and can include but are not limited to cubes, rectangles, rods, semi-circular rode and tubes. The solid composition of this disclosure can then be removed from the mold.

In an aspect, the compositions of this disclosure can be used to prepare an in-situ forming composition. A composition of this disclosure that contains available vinyl sulfone groups can be reacted with a compound that contains at least two available free thiol groups or a compound that contains at least 2 available amine groups, preferably primary or secondary amines. Illustrative thiol containing compounds include PEG-dithiol (HS-PEG-SH), 3-arm PEG-tri-thiol (glycerine core), 4-arm PEG-tetrathiol (pentaerythritol core), or 8-arm PEG-octa-thiol (hexaglycerine core). The foregoing multi-armed PEG reagents may also have fewer than all arms functionalized with thiol. Additional suitable thiol reagents having PEG as the central molecule are available from Laysan Bio (Arab, Ala.), as well as aromatic dithiols such as those available from NanoScience. Other suitable thiol crosslinkers include dimercaptosuccinic acid, 2,3-dimercapto-1-propanesulfonic acid, dihydrolipoic acid, peptides containing at least 2 cysteine amino acids, a thiol functionalized polysaccharide, thiol functionalized dextran, and thiol-functionalized hyaluronic acid.

The compositions of the present disclosure, e.g., the first, second and third derivatives of a starting polymer, may be in combination with one or more other compositions. Thus, the present disclosure provides compositions providing compositions of the present disclosure.

The compositions of this disclosure can be used to treat living organisms. These living organisms include humans, animals, birds, fish, insects and plants. The compositions used in the indications described below can comprise, non-crosslinked composition, crosslinked composition or a combination thereof. In an aspect, the compositions used can comprise only one of the compositions of this disclosure. In an aspect, the compositions used can comprise two or more of the compositions of this disclosure. The compositions can further comprise one or more excipients. The compositions can further comprise one or more biologically active agents. The compositions that are used in the indications described below can be in a sterile form. Sterilization can be attained through sterile filtration, aseptic manufacture, gamma radiation, e-beam radiation, ethylene oxide, dry heat, autoclaving, or a combination thereof. In an aspect, the compositions of this disclosure are sterilized using autoclaving. In an aspect, the temperature for autoclaving can be in the range of about 121° C. to about 130° C. In an aspect, the duration of the autoclave cycle can be in the range of 3 to 15 minutes.

For instance, the compositions of this disclosure can also comprise an excipient. The excipient may be a pharmaceutically acceptable excipient. Excipients that can be used include but are not limited to natural polymers, synthetic polymers, thermoreversible polymers, biodegradable polymers, buffers, complexing agents, tonicity modulators, ionic strength modifiers, solvents, anti-oxidants, preservatives, viscosity modifiers, pH modifiers, surfactants, emulsifiers, phospholipids, stabilizers and porogens.

Excipient polymers that can be used include but are not limited to sodium alginate, calcium alginate, dextran, carboxymethyl cellulose, hydroxypropyl cellulose, hydroxypropyl methyl cellulose, methyl cellulose, Hyaluronic acid, hyaluronic acid derivatives, dextran, heparin, chitosan, chitin, xantham gum, Xylan, guar gum, pullulan, locust bean gum, starch, gelatin, collagen, derivatized collagen, chondroitin Sulfate polymers, dermatan Sulfate polymers, keratan sulfate polymers, heparin, heparan sulfate, and acacia (gum Arabic).

Excipient degradable polymers that can be used include but are not limited to polyesters, polyether esters, polyorthoesters, poly ester carbonates, polycarbonates, polyanhydrides, polyhydroyalkonate (e.g. Polyhydroxybutyrate, polyhydroxyvalerates), polyurethanes, polyester urethanes. The polymers can be in the form of linear, branched, or star shaped. The polymers can be initiated from compounds that us a single point of initiation, two points of initiation, 3 points of initiation, four points of initiation, 6 points of initiation or 8 points of initiation. Polymers can include but are not limited to polymers that are comprise repeat units derived from at least one of the following monomers: l-lactide, dl-lactide, glycolide, trimethylene carbonate, epsilon-caprolactone, p-dioxanone and a morpholinedione

Excipient synthetic polymers that can be used include but are not limited to polyacrylic acid and salts thereof, polyvinylpyrollidone, pluronics 127, pluronics F68, polyethylene glycol, polyethylene oxide, and polyvinyl alcohol.

Complexing agents can include but are not limited to α-cyclodextrin, β-cyclodextrin, (2-Hydroxypropyl)-Beta-Cyclodextrin, sulfobutylether beta cyclodextrin, and ethylenediaminetetraacetic acid (EDTA) and salts thereof.

Phospholipids that can be used include but are not limited to hydrogenated soy phosphatidylcholine, distearoylphosphatidylglycerol, L-α-dimyristoylphosphatidylcholine, and L-α-dimyristoylphosphatidylglycerol

Surfactants that can be used include ionic and non-ionic surfactants. Ionic surfactants can include cationic, anionic and zwitterionic surfactants. Non-ionic surfactants can include but are not limited to (Cremophor EL, Cremophor RH 40, Cremophor RH 60, d-tocopherol polyethylene glycol 1000 succinate, Brij, Myrj, polysorbate 20, polysorbate 80, polysorbate 40, polysorbate 60, polysorbate 65, polysorbate 85, Solutol HS 15, sorbitan monooleate (Span 80), Sorbitan monopalmitate (Span 40), sorbitan monostearate (Span 60), sorbitan trioleate (Span 8) poloxamer 407, Labrafil M-1944CS, Labrafil M-2125CS, Labrasol, Gellucire 44/14, nonoxynol-9, Softigen 767, octyl beta-D-glycopyranoside (OGP), hexyl beta-D-glucopyranoside (HGP), Octyl beta-D-1-thioglucopyranoside (TGP), Decyl-beta-D-glucopyranoside (DGP), Dodecyl-beta-D-glucopyranoside (DdGP), N-octyl beta-D-Maltoside (ODM), decyl beta-D-maltopyranoside (DMP), cyclohexyl-ethanoyl-maltoside, n-decyl- and n-dodecyl-sucrose, and mono- and di-fatty acid esters of PEG 300, 400, or 1750. Anionic surfactants can include but are not limited to sodium lauryl sulfate, fatty acid salts, sodium laureth sulfate, dioctyl sodium sulfosuccinate. Cationic surfactants can include but are not limited to Phosphatidylcholine (Lecithin), cetrimide, cetrimonium bromide, benethonium chloride, dimethyldioctadecyl ammonium chloride, tetradecyl trimethyl ammonium bromide, cetylpyridinium chloride, esterquat, and benzalkonium chloride. Zwiterionic surfactants can include but are not limited to Cocamidopropyl betaine, (3-[(3-Cholamidopropyl)dimethylammonio]-1-propanesulfonate) and cocamidopropyl hydroxysultaine, phosphatidylserine, phosphatidylethanolamine, phosphatidylcholine, and sphingomyelins.

Solvents that can be used include water-soluble organic solvents. Water-soluble organic solvents include but are not limited to polyethylene glycol 200, polyethylene glycol 300, polyethylene glycol 400, ethanol, propylene glycol, glycerin, N-methyl-2-pyrrolidone, dimethylacetamide, and dimethylsulfoxide.

Tonicity modifiers that can be used include but are not limited to dextrose, sucrose, mannitol, glycerin, sodium chloride, and potassium chloride.

pH modifiers that can be used include but are not limited to citric acid and its salts, salts of phosphoric acid, tartatic acid, lactic acid, glycolic acid, sodium hydroxide, phosphoric acid, sulfuric acid, oxalic acid and hydrochloric acid.

Anti-oxidants that can be used include but are not limited to ascorbic acid, L-ascorbic acid, L-ascorbic acid 2-sulfate (AA-25) and L-ascorbic acid 2-phosphate (AA-2P), ascorbic acid 2-O-glucoside (AA-2G), 6-O-acyl-2-O-alpha-D-glucopyranosyl-L-ascorbic acids (6-Acyl-AA-2G), ascobyl 3-aminopropyl phosphate (Vitagen), Ascorbyl palmitate, butylated hydroxyanisole, Butylhydroxytoluene, Vitamin A, vitamin E, α-tocopherol, thioglycerol, cysteine, acetylcysteine, cystine, dimethylaminoethanol, dithioerythreitol, dithiothreitol, glutathione, alpha-lipoic acid, Sodium bisulfite, Sodium metabisulfite, thiourea, uric acid, melatonin, propyl gallate, tertiary butylhydroquinone, and combinations thereof, retinol (-hydroxyl group, —OH), tretinoin (retinoic acid-carboxyl acid group-COOH), and adapalence (carboxyl group, —COOH).

Emulsifiers that can be used include but are not limited to Glyceryl Monostearate, Isopropyl Palmitate, Polyethylene Glycol 400 Monostearate, as well as the compounds listed as surfactants and combinations thereof.

Preservatives that can be used include but are not limited to benzoic acid, sorbic acid, boric acid, methylparaben, ethylparaben, propylparaben, butylparaben, sodium benzoate, sodium propionate, phenyl ethyl alcohol, chlorobutanol, benzyl alcohol, potassium sorbate, phenol, chlorocresol, o-phenyl phenol, thiomersal, nitromersol, phenylmercuric nitrate, phenylmercuric acetate, benzalkonium and combinations thereof.

The excipients can include at least one solvent. The solvents used can include but are not limited to water, ethanol, dimethylsulfoxide, ethyl lactate, ethyl acetate, benzyl alcohol, benzyl benzoate, triacetin, N-methylpyrrolidone, 2-pyrrolidone, propylene carbonate, polyethylene glycol (PEG200), polyethylene glycol (PEG400), glycofurol and combinations thereof.

Buffers that can be used include aqueous solutions prepared using one or more of the following compositions: potassium hydrogen phthalate, sodium hydrogen phthalate, potassium or sodium dihydrogen phosphate, dipotassium or disodium hydrogen phosphate, phosphoric acid, boric acid, sodium acetate, acetic acid, ammonium chloride, ammonium acetate, (4-(2-hydroxyethyl)-1-piperazineethanesulfonic acid).

In an aspect, the compositions of this disclosure can further comprise an inorganic composition. The inorganic compositions that can be used include but are not limited to barium sulfate, calcium hydroxyapatite or hydroxyapatite, tricalcium phosphate (TCP) [including the various forms, for example α-TCP, β-TCP, and Biphasic Tricalcium Phosphate (BCP)], calcium phosphate, calcium sodium phosphosilicate, and calcium sulphate.

In an aspect, the compositions of this disclosure can further comprise hyaluronic acid or a salt thereof. In an aspect, the compositions of this disclosure can further comprise a derivative of hyaluronic acid as disclosed in this invention. In an aspect, the compositions of this disclosure can further comprise a mercaptobenzoic acid derivative of hyaluronic acid as disclosed herein.

Compositions of the disclosure can further comprise one or more polyol excipients. The polyol excipient can include but is not limited to sucrose, glycerol, erythritol, threitol, arabitol, erythritol, ribitol, xylitol, galactitol (or dulcitol), glucitol (or sorbitol), iditol, inositol, mannitol, isomalt, lactitol, maltitol, trehalose, dextrose, maltose and lactose, dextran, and polyglycitol. Other non-limiting examples of polyols can be found in, e.g., Pharmaceutical Dosage Forms and Drug Delivery Systems (Howard C. Ansel et al., eds., Lippincott Williams & Wilkins Publishers, 7″ ed. 1999); Remington: The Science and Practice of Pharmacy (Alfonso R. Gennaro ed., Lippincott, Williams & Wilkins, 20″ ed. 2000); Goodman & Gilman's The Pharmacological Basis of Therapeutics (Joel G. Hardman et al., eds., McGraw-Hill Professional, 10″ ed. 2001); and Handbook of Pharmaceutical Excipients (Raymond C. Rowe et al., APhA Publications, 4 edition 2003), each of which is hereby incorporated by reference in its entirety.

Compositions of the disclosure can further comprise one or more contrast agents. The contrast agent can result in the composition of the disclosure being visible under x-rays, including but not limited to fluoroscopy and computerized tomography (CT), magnetic resonance imaging (MRI), ultrasound or a combination thereof. In an aspect, the contrast agent can be ionic or nonionic. Contrast agents can include but are not limited to barium sulfate, iodine-based contrast agents, gadolinium-based contrast agents and microbubble contrast agents. Iodine-based contrast agents include but are not limited to diatrizoate sodium/meglumine, iothalamate sodium/meglumine, iopamidol, iohexol, iopromide, ioversol, ioxilan, iodixanol, ethiodized poppyseed oil (Lipiodol) or combinations thereof. Gadolinium-based contrast agents include but are not limited to gadodiamide, gadopentetate dimeglumine, gadoversetamide, gadobenate dimeglumine, gadobutrol, gadoterate meglumine, gadoteridol, gadoxetic acid disodium salt or combinations thereof. Microbubble contrast agents include but are not limited to Echovist®, albunex®, Levovist®, Optison®, Sonovue®, Definity®, Sonazoid® or combinations thereof.

In an aspect, the compositions of this disclosure can be prepared as a solution that comprises one or more excipients. In an aspect, the compositions of this disclosure can be suspended in a solution that comprises one or more excipients. In an aspect, the compositions of this disclosure can be rehydrated in a solution that comprises one or more excipients. In an aspect, the compositions of this disclosure can be prepared as separate solutions that can comprise one or more excipients with the separate solutions being mixed prior to use. In an aspect, the compositions of this disclosure can be prepared in the presence of one or more excipients and then converted to a solid form by one or more of the methods described in this disclosure.

Compositions of the present disclosure may include a biological agent in addition to a polymer as described herein and optionally other ingredients. Exemplary biologically active agents include, without limitation, small molecule drugs, peptides, proteins, growth factors, hormones, antibodies, agonists, antagonists, anti-bacterial and/or anti-fungal agents.

Biologically active agents that can be incorporated into formulations with Compositions described include: antiandrogens, antibacterial, antioestrogens, androgens and anabolic agents, antibiotics, antimigraine drugs, antihistamines, antianxiety drugs, antidiuretics, antihistamines, antirheumatoid agents, antigens, analgesics, antidepressants, antiinflammatories, anesthetics, aminoglycosides antibodies, antiviral, adrenergic stimulants, anticonvulsants, antiangina agents, antiarrhyrthmics, antimalarials, anti-mitotic, anthelmintics, anoretic agents, antitussives, antipruritics, antipyretics, anti-alzheimer's agents, anti-Parkinson's agents, antiemetics and antinauseants, antihypertensives, anticoagulants, antifungals, antimicrobials, allergens, antidiarrheals, antihyperuricaemia agents, adrenergic stimulants, antiparasitic agents, antiproliferative agents, antipsychotic drugs, antithyroid agents, beta-adrenergic blocking agents, bronchodilators; bronchospasm relaxants, blood clotting factors, blood coagulation factors, cytotoxic agents, cytostatic agents, chemotherapeutics, clot inhibitors, clot dissolving agents, cells, CNS stimulants, Corticosteroids, calcium channel blockers, cofactors, ceramides, cardiotonic glycosides, cytokines (e.g., lymphokines, monokines, chemokines); colony stimulating factors (e.g., GCSF, GM-CSF, MCSF); dermatological agents, decongestants, diuretics, expectorants, endectocide agents, growth factors, hemostatic agents, hypoglycemic agents, hormones and hormone analogs, hypercalcemia, Hypnotics, interleukins (IL-2, IL-3, IL-4, IL-6); interferons (.beta.-IFN, .alpha.-IFN and .gamma.-IFN); immunosuppressants, muscle relaxants, microorganisms, non-steroidal anti-inflammatory agents, nucleic acids, nutritional agents, neuromuscular blocking agents, neuroleptics, Neurotoxins, nutraceuticals, oligonucleotides, oestrogens, obstetric drugs, ovulation inducers, opioids, progestogens, pituitary hormones, Pituitary inhibitors proteins, peptides, polysaccharides, protease inhibitors, prostaglandins, quinolones, reductase inhibitors, sulfa drugs, sclerosant, sedatives, sodium channel blockers, steroids, steroidal anti-inflammatory agents, smoking cessation agents, toxins, thrombolytic agents, thyroid hormones, tumor necrosis factor; vesicles, vitamins, viruses, vasodilators, vaccines

Additional representative examples of biologically active agents that may be suitable for use in compositions of the present disclosure include, but are not limited to: Antidiarrheals such as diphenoxylate, loperamide and hyoscyamine; Antihypertensives such as hydralazine, minoxidil, captopril, enalapril, clonidine, prazosin, debrisoquine, diazoxide, guanethidine, methyldopa, reserpine, trimethaphan; calcium channel blockers such as diltiazem, felodipine, amlodipine, nitrendipine, nifedipine and verapamil; antiarrhyrthmics such as amiodarone, flecainide, disopyramide, procainamide, mexiletene and quinidine, antiangina agents such as glyceryl trinitrate, erythrityl tetranitrate, pentaerythritol tetranitrate, mannitol hexanitrate, perhexilene, isosorbide dinitrate and nicorandil; Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate; cardiotonic glycosides such as digoxin and other cardiac glycosides and theophylline derivatives; adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine; vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol; Antiproliferative agents such as paclitaxel, estradiol, actinomycin D, sirolimus, tacrolimus, everolimus, 5-fluorouracil and dexamethasone; antimigraine preparations such as ergotanmine, dihydroergotamine, methysergide, pizotifen and sumatriptan; anticoagulants and thrombolytic agents such as warfarin, dicoumarol, low molecular weight heparins such as enoxaparin, streptokinase and its active derivatives; hemostatic agents such as aprotinin, tranexamic acid and protamine; analgesics and antipyretics including the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine, dihydrocodeine; acetylsalicylic acid (aspirin), paracetamol, synthetic alpha2-adrenoreceptor agonist, dexmedetomidine hydrochloride, flunixin meglumine, meperidine, phenylbutazone and phenazone; Immunosuppressants, antiproliferatives and cytostatic agents such as rapamycin (sirolimus) and its analogs (everolimus and tacrolimus); neurotoxins such as capsaicin, botulinum toxin (botox); hypnotics and sedatives such as the barbiturates amylobarbitone, butobarbitone and pentobarbitone and other hypnotics and sedatives such as chloral hydrate, chlormethiazole, hydroxyzine and meprobamate; antianxiety agents such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam; neuroleptic and antipsychotic drugs such as the phenothiazines, chlorpromazine, fluphenazine, pericyazine, perphenazine, promazine, thiopropazate, thioridazine, trifluoperazine; and butyrophenone, droperidol and haloperidol; and other antipsychotic drugs such as pimozide, thiothixene and lithium; antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline; CNS stimulants such as caffeine and 3-(2-aminobutyl) indole; antipruritics can include compounds such as synthetic Janus Kinase (JAK) inhibitors, NK-1 receptor antagonists, antibodies that neutralize interleukin-31 (IL-31). These can include oclacitinib maleate, Serlopitant, and Lokivetmab, anti-alzheimer's agents such as tacrine; anti-Parkinson's agents such as amantadine, benserazide, carbidopa, levodopa, benztropine, biperiden, benzhexol, procyclidine and dopamine-2 agonists such as S (−)-2-(N-propyl-N-2-thienylethylamino)-5-hydroxytetralin (N-0923), anticonvulsants such as phenytoin, valproic acid, primidone, phenobarbitone, methylphenobarbitone and carbamazepine, ethosuximide, methsuximide, phensuximide, sulthiame and clonazepam, antiemetics and antinauseants such as the phenothiazines prochloperazine, thiethylperazine, a neurokinin (NK1) receptor antagonist, maropitant citrate and 5HT-3 receptor antagonists such as ondansetron and granisetron, as well as dimenhydrinate, diphenhydramine, metoclopramide, domperidone, hyoscine, hyoscine hydrobromide, hyoscine hydrochloride, clebopride and brompride; non-steroidal anti-inflammatory agents including their racemic mixtures or individual enantiomers where applicable, preferably which can be formulated in combination with dermal and/or mucosal penetration enhancers, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, 6-chloro-α-methyl-9H-carbazole-2-acetic acid (carprofen), flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate; Antirheumatoid agents such as penicillamine, aurothioglucose, sodium aurothiomalate, methotrexate and auranofin; muscle relaxants such as baclofen, diazepam, cyclobenzaprine hydrochloride, dantrolene, methocarbamol, orphenadrine and quinine; agents used in gout and hyperuricaemia such as allopurinol, colchicine, probenecid and sulphinpyrazone; oestrogens such as estradiol, oestriol, estrone, ethinylestradiol, mestranol, stilbestrol, dienestrol, epiestriol, estropipate and zeranol; Progesterone and other progestagens such as allylestrenol, dydrgesterone, lynestrenol, norgestrel, norethyndrel, norethisterone, norethisterone acetate, gestodene, levonorgestrel, medroxyprogesterone and megestrol; antiandrogens such as cyproterone acetate and danazol; antioestrogens such as tamoxifen and epitiostanol and the aromatase inhibitors, exemestane and 4-hydroxy-androstenedione and its derivatives; androgens and anabolic agents such as testosterone, methyltestosterone, clostebol acetate, drostanolone, furazabol, nandrolone oxandrolone, stanozolol, trenbolone acetate, dihydro-testosterone, 17-(.alpha.-methyl-19-noriestosterone and fluoxymesterone; 5-alpha reductase inhibitors such as finasteride, turosteride, LY-191704 and MK-306; corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone, triamcinolone acetonide; glycosylated proteins, proteoglycans, glycosaminoglycans such as chondroitin sulfate; chitin, acetyl-glucosamine, hyaluronic acid; Complex carbohydrates such as glucans; further examples of steroidal anti-inflammatory agents such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone, aincinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol; pituitary hormones and their active derivatives or analogs such as corticotrophin, thyrotropin, follicle stimulating hormone (FSH), a Gonadotropin-releasing hormone (GnRH) analog, deslorelin acetate, cetrorelix acetate, gonadorelin acetate, clomiphene, Human chorionic gonadotropin (HCG), luteinizing hormone (LH) and gonadotrophin releasing hormone (GnRH); hypoglycemic agents such as insulin, chlorpropamide, glibenclamide, gliclazide, glipizide, tolazamide, tolbutamide and metformin; thyroid hormones such as calcitonin, thyroxine and liothyronine and antithyroid agents such as carbimazole and propylthiouracil; other miscellaneous hormone agents such as octreotide; pituitary inhibitors such as bromocriptine; ovulation inducers such as clomiphene; Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potasium sparing diuretics, spironolactone, amiloride and triamterene; Antidiuretics such as desmopressin, lypressin and vasopressin including their active derivatives or analogs; Obstetric drugs including agents acting on the uterus such as ergometrine, oxytocin and gemeprost; prostaglandins such as alprostadil (PGE1), prostacyclin (PG12), dinoprost (prostaglandin F2-alpha) and misoprostol; antimicrobials including the cephalosporins such as cephalexin, cefoxytin and cephalothin; penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin; tetracyclines such as minocycline, chlortetracycline, tetracycline, demeclocycline, doxycycline, methacycline and oxytetracycline and other tetracycline-type antibiotics; Amnioglycoides such as amikacin, amikin sulfate, gentamicin, kanamycin, neomycin, netilmicin and tobramycin; antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione; quinolones such as nalidixic acid, cinoxacin, ciprofloxacin, enoxacin and norfloxacin; sulphonamides such as phthalysulphthiazole, sulfadoxine, sulphadiazine, sulphamethizole and sulphamethoxazole; Sulphones such as dapsone; other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonarn, colistin IV, metronidazole, tinidazole, secnidazole, ornidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroamine compounds, silver sulfadiazine, silver, nanoparticulate silver, silver nitrate, silver zeolites, silver cations, AgPO3 Ag3PO4, Ag4P2O7, exsalt® SD7 (Exciton Technologies) exsalt® T7 (Exciton Technologies); Lincomycin Hydrochloride, tricyclic tetrahydroquinoline antibacterial agents, 8-pyrazinyl-S-spiropyrimidinetrione-oxazinoquinoline derivatives, 3-spiropyrimidinetrione-quinoline derivatives, thiadiazol-spiropyrimidinetrione-quinoline derivatives, (2R,4S,4aS)-10-fluoro-2,4-dimethyl-8-(4-methyloxazol-2-yl)-2,4,4a,6-tetra-hydro-1H,1′H-spiro[[1,4]oxazino[4,3-a]quinoline-5,5′-pyrimidine]-2′,4′,6′(-3′H)-trione, (2R,4S,4aS)-9,10-difluoro-2,4-dimethyl-8-(3-methylisoxazol-5-yl)-2,4,4a,6-tetrahydro-1H,1′H-spiro[[1,4]oxazino[4,3-a]quinoline-5,5′-pyrimidine]-2′,-4′,6′(3′H)-trione, (2R,4S,4aS)-10-fluoro-2,4-dimethyl-8-(oxazol-2-yl)-2,4,4a,6-tetrahydro-1H-,1′H-spiro[[1,4]oxazino[4,3-a]quinoline-5,5′-pyrimidine]-2′,4′,6′(3′H)-tri-one, (2R,4S,4aS)-9,10-difluoro-2,4-dimethyl-8-(2-methyloxazol-5-yl)-2,4,4a,6-t-etrahydro-1H,1′H-spiro[[1,4]oxazino[4,3-a]quinoline-5,5′-pyrimidine]-2′,4′-,6′(3′H)-trione, (2R,4S,4aS)-9,10-difluoro-2,4-dimethyl-8-(oxazol-4-yl)-2,4,4a,6-tetrahydr-o-1H,1′H-spiro[[1,4]oxazino[4,3-a]quinoline-5,5′-pyrimidine]-2′,4′,6′(3′H)-trione, (2R,4S,4aS)-9-fluoro-2,4-dimethyl-8-(4-methyloxazol-2-yl)-2,4,4a,6-tetrah-ydro-1H,1′H-spiro[[1,4]oxazino[4,3-a]quinoline-5,5′-pyrimidine]-2′,4′,6′(3′H)-trione, (2R,4S,4aS)-9,10-difluoro-8-(4-(4-fluorophenyl)oxazol-5-yl)-2,4-dimethyl-2,4,4a,6-tetrahydro-1H,1′H-spiro[[1,4]oxazino[4,3-a]quinoline-5,5′-pyrimid-ine]-2′,4′,6′(3′H)-trione, (2S,4R,4aR)-2,4-dimethyl-8-(oxazol-5-yl)-2,4,4a,6-tetrahydro-1H,1′H-spiro-[[1,4]oxazino[4,3-a]quinoline-5,5′-pyrimidine]-2′,4′,6′(3′H)-trione, (2S,4R,4aR)-8-(4-ethyloxazol-2-yl)-9,10-difluoro-2,4-dimethyl-2,4,4a,6-te-trahydro-1H,1′H-spiro[[1,4]oxazino[4,3-a]quinoline-5,5′-pyrimidine]-2′,4′,-6′(3′H)-trione, (2R,4S,4aS)-9,10-difluoro-2,4-dimethyl-8-(oxazol-2-yl)-2,4,4a,6-tetrahydr-o-1H,1′H-spiro[[1,4]oxazino[4,3-a]quinoline-5,5′-pyrimidine]-2′,4′,6′(3′H)-trione, benzoyl peroxide; Antituberculosis drugs such as ethambutol, isoniazid, pyrazinamide, rifampicin and clofazimine; Antimalarials such as primaquine, pyrimethamine, chloroquine, hydroxychloroquine, quinine, mefloquine and halofantrine; compounds such as Azithromycin, Aztreonam, Cefaclor, Cefadroxil, Cefazolin, Cefdinir, Cefepime Hydrochloride, (cefoperazone sodium, Ceftaroline fosamil, avibactam, Ceftazidime sodium, Ceftibuten, ceftiofur, Tazobactam, cefovecin sodium [(6R,7R)-7-[[(2Z)-(2-amino-4-thiazolyl)(methoxyimino)acetyl]amino]-8-oxo-3-[(2S)-tetrahydro-2-furanyl]-5-thia-1-azabicyclo[4.2.0]oct-2-ene-2-carboxylic acid, monosodium salt] Cefuroxime Axetil, Cefuroxime, Cephalexin, Chloramphenicol Sodium, Ciprofloxacin HCl, Clarithromycin, Clindamycin hydrochloride, Clindamycin Palmitate hydrochloride, Clindamycin phosphate, Dalbavancin Hydrochloride, Daptomycin, Demeclocycline hydrochloride, Dicloxacillin, Doripenem, Doxycycline, Doxycycline calcium, Doxycycline hyclate, Doxycycline monohydrate, Ertapenem sodium, Erythromycin, Erythromycin Ethylsuccinate, Erythromycin lactobionate, Erythromycin stearate, Erythromycin, Fosfomycin tromethamine, Gemifloxacin mesylate, Gentamicin Sulfate, Imipenem, Kanamycin, Levofloxacin, Lincomycin hydrochloride, Linezolid, Meropenem, Methenamine Hippurate, Metronidazole, Metronidazole, Micafungin sodium, Minocycline Hydrochloride, Minocycline, Moxifloxacin hydrochloride, Nafcillin, Nalidixic acid, Neomycin Sulfate, Nitrofurantoin, Norfloxacin, Ofloxacin, Oritavancin diphosphate, Oxacillin, Penicillin G, Penicillin G benzathine, Penicillin G Sodium, Penicillin V Potassium, Piperacillin Sodium, Polymyxin B Sulfate, Quinupristin, dalfopristin, Spectinomycin hydrochloride, Streptomycin, Sulfamethoxazole, Tedizolid Phosphate, Telavancin, Telithromycin, Tetracycline Hydrochloride, Ticarcillin disodium, Tigecycline, Tobramycin Sulfate, Tobramycin, Trimethoprim hydrochloride, tulathromycin, Vancomycin hydrochloride.

Antiviral agents may be included in compositions of the present disclosure, where exemplary antiviral agents include acyclovir and acyclovir prodrugs, famcyclovir, zidovudine, didanosine, stavudine, lamivudine, zalcitabine, saquinavir, indinavir, ritonavir, n-docosanol, tromantadine and idoxuridine. Other suitable biologically active agents include anthelmintics such as mebendazole, thiabendazole, niclosamide, praziquantel, pyrantel embonate and diethylcarbamazine; cytotoxic agents such as plicamycin, cyclophosphamide, dacarbazine, fluorouracil and its prodrugs (described, for example, in International Journal of Pharmaceutics, 111, 223-233 (1994)), methotrexate, procarbazine, 6-mercaptopurine and mucophenolic acid; Anorectic and weight reducing agents including dexfenflurarnine, fenfluramine, diethylpropion, mazindol and phentermine; agents used in hypercalcaemia such as calcitriol, dihydrotachysterol and their active derivatives or analogs; Antitussives such as ethylmorphine, dextromethorphan and pholcodine; antiparasitic and endectocide agents such as moxidectin, Ivermectin, Niclosamide, Praziquantel, Pyrantel, Pyrvinium, Albendazole, Flubendazole, Mebendazole, Thiabendazole

Compositions of the present disclosure may include: an expectorant such as carbolcysteine, bromihexine, emetine, quanifesin, ipecacuanha and saponins; Decongestants such as phenylephrine, phenylpropanolamine and pseudoephedrine; Bronchospasm relaxants such as ephedrine, fenoterol, orciprenaline, rimiterol, salbutamol, sodium cromoglycate, cromoglycic acid and its prodrugs (described, for example, in International Journal of Pharmaceutics 7, 63-75 (1980)), terbutaline, ipratropium bromide, salmeterol and theophylline and theophylline derivatives; Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine; Local anaesthetics such as benzocaine, bupivacaine, amethocaine, lignocaine, lidocaine, cocaine, cinchocaine, dibucaine, mepivacaine, prilocaine, etidocaine, veratridine (specific c-fiber blocker) and procaine; Stratum corneum lipids, such as ceramides, cholesterol and free fatty acids, for improved skin barrier repair [Man, et al. J. Invest. Dermatol., 106(5), 1096, (1996)]; Neuromuscular blocking agents such as suxamethonium, alcuronium, pancuronium, atracurium, gallamine, tubocurarine and vecuronium; sclerocing agents or sclerosants may be a surfactant or it may be selected from the group consisting of ethanol, dimethyl sulfoxide, sucrose, sodium chloride, dextrose, glycerin, minocycline, tetracycline, doxycycline, polidocanol, sodium tetradecyl sulfate, sodium morrhuate, and sotradecol. an angiogenesis inhibitor; a 5-lipoxygenase inhibitor or antagonist; a chemokine receptor antagonist; a cell cycle inhibitor; a taxane; an anti-microtubule agent; paclitaxel; an analogue or derivative of paclitaxel; a vinca alkaloid; camptothecin or an analogue or derivative thereof; a podophyllotoxin, wherein the podophyllotoxin may be an etoposide or an analogue or derivative thereof; an anthracycline, wherein the anthracycline may be doxorubicin or an analogue or derivative thereof, mitoxantrone or an analogue or derivative thereof or epirubicin or an analogue or derivative thereof; a platinum compound; a nitrosourea; a nitroimidazole; a folic acid antagonist; a cytidine analogue; a pyrimidine analogue; a fluoropyrimidine analogue; a purine analogue; a nitrogen mustard or an analogue or derivative thereof; a hydroxyurea; a mytomicin or an analogue or derivative thereof; an alkyl sulfonate; a benzamide or an analogue or derivative thereof; a nicotinamide or an analogue or derivative thereof; a halogenated sugar or an analogue or derivative thereof; a DNA alkylating agent; an anti-microtubule agent; a topoisomerase inhibitor; a DNA cleaving agent; an antimetabolite; a nucleotide interconversion inhibitor; a hydroorotate dehydrogenase inhibitor; a DNA intercalation agent; an RNA synthesis inhibitor; a pyrimidine synthesis inhibitor; a cyclin dependent protein kinase inhibitor; an epidermal growth factor kinase inhibitor; an elastase inhibitor; a factor Xa inhibitor; a farnesyltransferase inhibitor; a fibrinogen antagonist; a guanylate cyclase stimulant; a heat shock protein 90 antagonist; which may be a geldanamycin or an analogue or derivative thereof; a guanylate cyclase stimulant; a HMGCoA reductase inhibitor, which may be simvastatin or an analogue or derivative thereof; an IKK2 inhibitor; an IL-1 antagonist; an ICE antagonist; an IRAK antagonist; an IL-4 agonist; an immunomodulatory agent; sirolimus or an analogue or derivative thereof; everolimus or an analogue or derivative thereof; tacrolimus or an analogue or derivative thereof; biolmus or an analogue or derivative thereof; tresperimus or an analogue or derivative thereof; auranofin or an analogue or derivative thereof; 27-O-demethylrapamycin or an analogue or derivative thereof; gusperimus or an analogue or derivative thereof; pimecrolimus or an analogue or derivative thereof; ABT-578 or an analogue or derivative thereof; an inosine monophosphate dehydrogenase (IMPDH) inhibitor, which may be mycophenolic acid or an analogue or derivative thereof or 1-.alpha.-25 dihydroxy vitamin D.sub.3 or an analogue or derivative thereof; a leukotriene inhibitor; an MCP-1 antagonist; an MMP inhibitor; an NF kappa B inhibitor, which may be Bay 11-7082; an NO antagonist; a p38 MAP kinase inhibitor, which may be SB 202190; a phosphodiesterase inhibitor; a TGF-beta inhibitor; a thromboxane A2 antagonist; a TNF-.alpha. antagonist; a TACE inhibitor; a tyrosine kinase inhibitor; vitronectin inhibitor; a fibroblast growth factor inhibitor; a protein kinase inhibitor; a PDGF receptor kinase inhibitor; an endothelial growth factor receptor kinase inhibitor; a retinoic acid receptor antagonist; a platelet derived growth factor receptor kinase inhibitor; a fibrinogen antagonist; an antimycotic agent; sulconizole; a bisphosphonate; a phospholipase A1 inhibitor; a histamine H1/H2/H3 receptor antagonist; a macrolide antibiotic; a GPIlb/Illa receptor antagonist; an endothelin receptor antagonist; a peroxisome proliferator-activated receptor agonist; an estrogen receptor agent; a somastostatin analogue; a neurokinin 1 antagonist; a neurokinin 3 antagonist; a VLA-4 antagonist; an osteoclast inhibitor; a DNA topoisomerase ATP hydrolyzing inhibitor; an angiotensin I converting enzyme inhibitor; an angiotensin II antagonist; an enkephalinase inhibitor; a peroxisome proliferator-activated receptor gamma agonist insulin sensitizer; a protein kinase C inhibitor; a ROCK (rho-associated kinase) inhibitor; a CXCR3 inhibitor; Itk inhibitor; a cytosolic phospholipase A.sub.2-.alpha. inhibitor; a PPAR agonist; an immunosuppressant; an Erb inhibitor; an apoptosis agonist; a lipocortin agonist; a VCAM-1 antagonist; a collagen antagonist; an .alpha.-2 integrin antagonist; a TNF-.alpha. inhibitor; a nitric oxide inhibitor; and a cathepsin inhibitor. anti-fibrin and fibrinolytic agents, including plasmin, streptokinase, single chain urokinase, urokinase, t-PA (tissue type plasminogen activator), aminocaproic acid; anti-platelet agents including, aspirin, prostacyclins (and analogues); glycoprotein Ilb/Ilila agents including monoclonal antibodies, peptides (e.g. ReoPro, Cilastagel, eptifibatide, tirofiban, ticlopidine, Vapiprost, dipyridamole, forskolin, angiopeptin, argatroban), thromboxane inhibitors; anti-thrombin and anti-coagulant agents, including dextan, heparin, LMW heparin (Enoxaparin, Dalteparin), hirudin, recombinant hirudin, anti-thrombin, synthetic antithrombins, thrombin inhibitors, Warfarin (and other coumarins); anti-mitotic, antiproliferative and cytostatic agents, including vincristine, vinblastine, paclitaxel, methotrexate, cisplatin, carboplatin, oxaliplatin, fluorouracil, rapamycin, azathioprine, cyclophosphamide, mycophenolic acid, corticosteroids, colchicine, nitroprusside; antiangiogenic and angiostatic agents, including paclitaxel, angiostatin and endostatin; genetic compositions and oligonucleotides; ACE inhibitors (e.g. Cilazapril, Lisinopril, Captopril); growth factor (e.g. VEGF, FGF) antagonists; antioxidants and vitamins (e.g. Probucol, Tocopherol); calcium channel blockers (e.g. nifedipine); fish oil (omega 3-fatty acid); phosphodiesterase inhibitors (e.g. dipyridamole); nitric acid donor (e.g. Molsidomine); somatostatin analogues (e.g. angiopeptin); immunosuppresives and anti-inflammatory agents (e.g. prednisolone, glucocorticoid and dexamethasone); antimicrobials (e.g. rifamycin) and radionuclides, including alpha, beta and gamma emitting isotopes (e.g. Re-188, Re-186, 1-125, Y-90); COX-2 inhibitors such as Celecoxib and Vioxx; kinase inhibitors, such as epidermal growth factor kinase inhibitor, tyrosine kinase inhibitors, MAP kinase inhibitors protein transferase inhibitors, Resten-NG, Smoking cessation agents such as nicotine, bupropion and ibogaine; Insecticides and other pesticides which are suitable for local application; Dermatological agents, such as vitamins A, C, B1, B2, B6, B 12, B 12.alpha., and E, vitamin E acetate and vitamin E sorbate; Allergens for desensitisation such as house, dust or mite allergens; Nutritional agents and neutraceuticals, such as vitamins, essential amino acids and fats; Macromolecular pharmacologically active agents such as proteins, enzymes, peptides, polysaccharides (such as cellulose, amylose, dextran, chitin), nucleic acids, cells, tissues, and the like; Bone mending biochemicals such as calcium carbonate, calcium phosphate, tricalcium phosphate, hydroxyapetite or bone morphogenic protein (BMP); Angiogenic growth factors such as Vascular Endothelial Growth Factor (VEGF) and epidermal growth factor (EFG), cytokines interleukins, fibroblasts and cytotaxic chemicals; and Keratolytics such as the alpha-hydroxy acids, glycolic acid and salicylic acid; and DNA, RNA or other oligonucleotides. Vaccines that contain Hendra virus (HeV) G glycoprotein and/or Nipah virus G glycoprotein, Lutenising Hormone Releasing Hormone (LHRH) peptide, LHRH-diphtheria toxoid conjugate, porcine circovirus type 2 (PCV2) antigen, a porcine reproductive and respiratory syndrome virus antigen, Mycoplasma hyopneumoniae protein antigen. Proteins or protein fragments, for example ORFI Torque teno virus protein, or other TTV proteins or fragments, antigens against Aeromonas salmonicida, antigens against Vibrio anguillarum, and antigens against V. salmonicida. Bone morphogenic proteins include but are not limited toBMP1, BMP2, BMP3, BMP4, BMP5, BMP6, BMP7, BMP8a, BMP8b, BMP9, BMP10, BMP11, BMP12, BMP13, BMP14 and BMP15. Growth factors include but are not limited to Vascular Endothelial Growth Factor (VEGF) and epidermal growth factor (EFG), Growth Differentiation Factors (GDFs), Fibroblast Growth Factors (FGF-1 through FGF-23), Osteoprotegerin, Cartilage Derived Morphogenic Proteins (CDMPs, which can be a foundation for soft or hard tissue), Lim Mineralization Proteins (LMPs) Interleukins (IL-1 through IL-13), Insulin-like Growth Factor-1, Connective Tissue Growth Factor (CTGF), platelet derived growth factor (PDGF), nerve growth factors, neutrophins Brain-derived neurotrophic factor (BDNF), Nerve growth factor (NGF), Neurotrophin-3 (NT-3), Neurotrophin-4 (NT-4)], Transforming growth factors (TGF-α, TGF-β), Tumor necrosis factor (TNF). Growth factor Agonists or antagonists as well as antibodies against these growth factors. Biologically active agents that can be used to treat macular degeneration include but are not limited to bevacizumab and ranibizumab. Other biologically active agents can include but are not limited to epidermal growth factor receptor tyrosine kinase inhibitor, retinol, tretinoin, mitomycin-C, a RANK Ligand inhibitor, a PDE4 inhibitor, a erythropoiesis stimulating protein, a proteasome inhibitor, a PCSK9 inhibitor, a PD-1 inhibitor, a IL-12 and/or IL-23 inhibitor, an anti-CD20 compound, an Inhibitor of Bruton's tyrosine kinase (Btk), a compound that inhibits IL-4 and interleukin-13 (IL-13), a dipeptidyl peptidase-4 (DPP-4) inhibitor, a glucagon-like peptide-1 receptor agonist (GLP-1 agonist), CD-38 inhibitor, a IL-17A inhibitor, a compound that inhibits the dimerization of HER2 with other HER receptors and adapalence. Epidermal growth factor receptor tyrosine kinase inhibitors can include but are not limited to osimertinib, olmutinib, lazertinib, mavelertinib, avitinib, rociletinib, nazartinib, naquotinib, erlotinib, gefitinib, afatinib, and dacomitinib.

In an aspect, compositions of the present disclosure are formulated for, and are useful for, wound healing. Compositions may be formulated for suitable administration, e.g., nasal ortopical administration. Compositions may include one or more suitable biologically active agents for wound healing. The wounds treated can include but are not limited to diabetic ulcers, burns, pressure wounds, abrasions, incisions, corneal abrasion, incisions following ocular surgery, blisters, damaged tissue following sinus surgery, abdominal surgery, tendon repair orjoint repair.

In an aspect, a composition of the disclosure can be in the form of dry particles. In an aspect, the composition of the disclosure can be in the form of a lyophilized composition. In an aspect, a composition comprises a fiber comprising polymers disclosed herein. In an aspect, a polymer composition of the disclosure can be in the form of a non-woven composition. In an aspect, the non-woven composition can be produced by an electrospinning process. In an aspect, the non-woven composition can be produced by wet-spinning process. In an aspect, the composition of the disclosure can be in the form of a film. These compositions can be packaged directly in indirectly in a foil pouch to minimize moisture absorption during storage.

Compositions of the disclosure that can be applied directly to a wound site. The compositions can absorb exudate from the wound. Once sufficient exudate is absorbed, the dry composition will turn into a gel. In an aspect, the compositions of the disclosure further comprise water or saline such that a gel is obtained. In an aspect, the gel can be applied directly to the wound.

In an aspect, compositions of the disclosure, once applied to the wound, can be covered by a have a moisture retaining semi-permeable film. The film can further comprise an adhesive that will retain the film at the site of application. The moisture retaining semi-permeable adhesive film can be made from a polyurethane or a silicone composition with an adhesive coating on at least the border or edges of the film. In an aspect, the adhesive can be an acrylic based adhesive. The semi-permeable film is permeable to oxygen and carbon dioxide, as well as water vapor but will prevent bacterial transmission.

In an aspect, the compositions of the disclosure can be applied to a semi-permeable film such than product is premade and ready to use in than composition of the disclosure and the semipermeable film are a single unit. This composition can be packaged directly or indirectly in a foil pouch. In an aspect, the composition of the disclosure may be used, for example, in a composition intended for wound healing.

In an aspect, compositions of the disclosure can be used as bulking agents. These bulking agents can be used to treat stress urinary incontinence, fecal incontinence, gastroesophageal reflux disease (GERD), or as a tissue spacer. In an aspect, the compositions of the disclosure can be used to increase the distance between a target organ or tissue that is to be exposed to radiotherapy treatment and one or more adjacent organs or tissues that are at risk of radiation exposure. In as aspect, the tissue spacer can be a prostate-rectum spacer for use on the reduction of rectal damage as a result of radiation treatment for prostate cancer. In an aspect, the tissue spacer can be a pancreas-duodenum spacer. In an aspect, the tissue spacer can be a breast-skin spacer. In an aspect, the tissue spacer can be a lymph node-esophagus spacer. In an aspect, the injected composition can be in the form of composition that comprises crosslinks. In an aspect, the bulking agent or tissue spacer can be injected through a needle. In an aspect, the needle used to inject the bulking agent or tissue spacer can be a 16G, 17G, 18G, 19G, 20G, 21G, 22G, 23G, 24G, 25G, 26G, 27G or a 30G needle. In an aspect, the needle used to inject the bulking agent or tissue spacer is a 18G. In an aspect, the needle used to inject the bulking agent or tissue spacer is a 21G. In an aspect, the needle used to inject the bulking agent or tissue spacer is a 23G.

In an aspect, the compositions of the disclosure can be used as a dermal filler to fill voids, defects and to treat moderate to severe wrinkles and folds. The compositions can be injected as a solution or suspension. In an aspect, the composition is crosslinked. In an aspect, a crosslinked composition of this disclosure that is used to manufacture a dermal filler, has an in-vitro hyaluronidase degradation profile that is the slower than that of a crosslinked unmodified hyaluronic acid polymer. In an aspect, a crosslinked composition of this disclosure that is used to manufacture a dermal filler, has an in-vitro hyaluronidase degradation profile that is the slower than that of a crosslinked unmodified hyaluronic acid polymer that is crosslinked under similar crosslinking conditions to a crosslinked composition of this disclosure.

Compositions disclosed herein can be used treat areas such as nasolabial folds and vertical lip lines. In an aspect, the crosslinked composition can be used to smooth brow furrows, crow's feet, frown lines, under eye troughs, marionette lines, chin wrinkles and neck wrinkles. In an aspect, the compositions can be used for lip augmentation, hand augmentation and breast augmentation. In an aspect, compositions disclosed herein can be injected in one or more of these locations in the tissue: superficially in the dermis, deep in the dermis, subcutaneously, or deep subcutaneously.

In an aspect, the compositions used as dermal fillers can further comprise a drug to reduce pain associated with the procedure. Such compounds include benzocaine, bupivacaine, amethocaine, lignocaine, lidocaine, cocaine, cinchocaine, dibucaine, mepivacaine, prilocaine, etidocaine, veratridine (specific c-fiber blocker) and procaine. The drug can be present in the final composition at a concentration of about 0.05% to about 5%. In an aspect, the drug is present at about 0.2% to about 0.5%. In an aspect, the drug is lidocaine. In an aspect, greater that 60% of the lidocaine is released within 20 hrs as measured by placing a sample of the lidocaine loaded composition into a dialysis tube, placing it in an aqueous medium and measuring the lidocaine in an aqueous medium. In an aspect, greater that 80% of the lidocaine is released within 20 hrs. In an aspect, compositions comprising lidocaine are stable over a period of at least 3 months at about 20° C. to about 25° C. wherein the lidocaine content does not change by more than 10% and that the viscosity of the composition does not change by more than 10%. In an aspect, the dermal filler can further comprise a degradable water-insoluble polymer (e.g. polyester such as PLGA, PLLA etc), a water insoluble non-degradable polymer (e.g. polymethylmethacrylate [PMMA]) or inorganic composition (e.g. calcium hydroxyapatite or calcium phosphate). In an aspect, the degradable water-insoluble polymer, a water insoluble non-degradable polymer or the inorganic composition can be in the form of particles. In an aspect, the particles can the regular in shape or irregular in shape.

In an aspect, compositions of the disclosure are formed into a crosslinked gel. The formed gel can be further processed such that a cohesive gel is obtained. This cohesive gel can be used as a dermal filler. This cohesive gel can be a monophasic gel. In an aspect, the monophasic gel can be used as a dermal filler. In an aspect, compositions are in the form of discrete particles of a crosslinked hydrogel. In an aspect, median size (Dv50) of the particles are in the range of 100 m to 800 m. In an aspect, the median size (Dv50) of the particles are in the range of 200 μm to 600 μm. In an aspect, the crosslinked hydrogel particles are suspended in a saline solution.

A dermal filler can further comprise a non-crosslinked polymer. In an aspect, the non-crosslinked polymer is hyaluronic acid, a salt of hyaluronic acid, a mercaptobenzoic acid derivative of hyaluronic acid or a combination thereof. In an aspect, the mercaptobenzoic acid derivative of hyaluronic acid comprises the reaction product of a vinyl sulfone functionalized hyaluronic acid with a mercaptobenzoic acid.

A dermal filler can comprise less that about 1% (w/w) of the non-crosslinked polymer relative to the crosslinked polymer of the disclosure. In an aspect, the dermal filler can comprise between about 1% (w/w) and about 50% (w/w) non-crosslinked polymer relative to the crosslinked polymer of the disclosure. In an aspect, the dermal filler can comprise between about 1% (w/w) and about 10% (w/w) non-crosslinked polymer relative to the crosslinked polymer of the disclosure. In an aspect, the dermal filler can comprise between about 10% (w/w) and about 20% (w/w) non-crosslinked polymer relative to the crosslinked polymer of the disclosure. In an aspect, the dermal filler can comprise between about 20% (w/w) and about 50% (w/w) non-crosslinked polymer relative to the crosslinked polymer of the disclosure.

A composition that comprises both crosslinked particles and non-crosslinked polymer is a biphasic composition. The biphasic composition can be used as a dermal filler.

In an aspect, the monophasic or biphasic compositions are in a prefilled syringe in which the contents of the syringe are sterile. In an aspect, monophasic or biphasic compositions are injectable through at least a 27G needle. In an aspect, the monophasic or biphasic compositions are injectable through a 27G needle with an average force of less than about 50 N when measured at an extrusion rate of about 12 mm per minutes. In an aspect, the monophasic or biphasic compositions are injectable through a 27G needle with an average force of less than about 40 N when measured at an extrusion rate of about 12 mm per minutes. In an aspect, the monophasic or biphasic compositions are injectable through a 27G needle with an average force of less than about 30 N when measured at an extrusion rate of about 12 mm per minutes. In an aspect, the monophasic or biphasic compositions are injectable through a 27G needle with an average force of less than about 20 N when measured at an extrusion rate of about 12 mm per minutes. In an aspect, the monophasic or biphasic compositions are injectable through a 27G needle with an average force of between about 5 N and about 20 N when measured at an extrusion rate of about 12 mm per minutes. In an aspect, the monophasic or biphasic compositions are injectable through a 27G needle with an average force of between about 20 N and about 40 N when measured at an extrusion rate of about 12 mm per minutes. In an aspect, the monophasic or biphasic compositions are injectable through a 30G needle.

A composition of the current disclosure can have a concentration of the derivatized hyaluronic acid, or salt thereof, in the range of about 5 mg/mL to about 60 mg/mL. In an aspect, the concentration of the derivatized hyaluronic acid, or salt thereof, in the range of about 10 mg/mL to about 40 mg/mL. In an aspect, the concentration of the derivatized hyaluronic acid, or salt thereof, in the range of about 15 mg/mL to about 30 mg/mL.

A composition of the current disclosure can have a concentration of the crosslinked derivatized hyaluronic acid, or salt thereof, in the range of about 5 mg/mL to about 60 mg/mL. In an aspect, the concentration of the crosslinked derivatized hyaluronic acid, or salt thereof, in the range of about 10 mg/mL to about 40 mg/mL. In an aspect, the concentration of the crosslinked derivatized hyaluronic acid, or salt thereof, in the range of about 15 mg/mL to about 30 mg/mL. For the composition of the current disclosure that comprise the crosslinked derivatized hyaluronic acid, or salt thereof, and a non-crosslinked polymer, the total concentration is the sum of the concentration of the crosslinked derivatized hyaluronic acid, or salt thereof, and a non-crosslinked polymer. The total concentration can be in the range of about 5 mg/mL to about 60 mg/mL. In an aspect, the total concentration can be in the range of about 10 mg/mL to about 40 mg/mL. In an aspect, the total concentration can be in the range of about 15 mg/mL to about 30 mg/mL.

The rheological properties of a composition of the disclosure can be measured using a rheometer. The measured properties are the elastic or storage modulus (G′), the viscous or loss modulus (G″) and tan 6 (G″/G′). As used herein, elastic modulus and storage modulus can be used interchangeably, as well as viscous modulus and loss modulus can be used interchangeably. In an aspect the composition of the disclosure can have an elastic or storage modulus of about 10 Pa to about 15,000 Pa. In an aspect the composition of the disclosure can have an elastic modulus of about 40 Pa to about 3,000 Pa. In an aspect a composition of the disclosure can have an elastic modulus of about 50 Pa to about 1,000 Pa. In an aspect the composition of the disclosure can have an elastic modulus of about 100 Pa to about 800 Pa. In an aspect a composition of the disclosure can have an elastic modulus of about 200 Pa to about 600 Pa. In an aspect a composition of the disclosure can have an elastic modulus of about 400 Pa to about 800 Pa. In an aspect, a composition of the disclosure can have an elastic modulus of about 25 Pa, about 50 Pa, about 75 Pa, about 100 Pa, about 125 Pa, about 150 Pa, about 175 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, about 800 Pa, about 850 Pa, about 900 Pa, about 950 Pa, about 1,000 Pa, about 1,200 Pa, about 1,300 Pa, about 1,400 Pa, about 1,500 Pa, about 1,600 Pa, about 1700 Pa, about 1800 Pa, about 1900 Pa, about 2,000 Pa, about 2,100 Pa, about 2,200 Pa, about 2,300 Pa, about 2,400 Pa, or about 2,500 Pa.

In an aspect the composition of the disclosure can have a viscous or loss modulus of about 3 Pa to about 1,500 Pa. In an aspect the composition of the disclosure can have a viscous modulus of about 10 Pa to about 1,000 Pa. In an aspect the composition of the disclosure can have a viscous modulus of about 15 Pa to about 500 Pa. In an aspect the composition of the disclosure can have a viscous modulus of about 20 Pa to about 250 Pa. In an aspect the composition of the disclosure can have a viscous modulus of about 3 Pa, about 5 Pa, about 10 Pa, about 20 Pa, about 30 Pa, about 40 Pa, about 50 Pa, about 60 Pa, about 70 Pa, about 80 Pa, about 90 Pa, about 100 Pa, about 150 Pa, about 200 Pa, about 250 Pa, about 300 Pa, about 350 Pa, about 400 Pa, about 450 Pa or about 500 Pa.

In an aspect the composition of the disclosure can have tan 6 of about 0.05 Pa to about 0.6 Pa. In an aspect the composition of the disclosure can have tan 6 of about 0.1 Pa to about 0.5 Pa. In an aspect the composition of the disclosure can have tan 6 of about 0.15 Pa to about 0.5 Pa. In an aspect the composition of the disclosure can have tan 6 of about 0.15 Pa to about 0.35 Pa.

Compositions of the disclosure that are packaged in a syringe have an extrusion force that is required to expel the composition from the syringe. For the composition in a 1 mL syringe with a 27 gauge needle attached that is expelled by pushing the plunger of the syringe at a rate of 12 mm/minute, the average extrusion force is about 8N to about 60N. In an aspect, the average extrusion force is about 12N to about 50N. In an aspect, the average extrusion force is about 15N to about 40N.

Compositions of the disclosure can be administered to the intended treatment site. The amount of the composition administered is, about 0.01 g, about 0.05 g, about 0.1 g, about 0.5 g, about 1 g, about 5 g, about 10 g, about 20 g, about 30 g, about 40 g, about 50 g, about 60 g, about 70 g, about 80 g, about 90 g, about 100 g, or about 150 g. In an aspect, the amount of the composition administered is about 0.01 g to about 0.1 g, about 0.1 g to about 1 g, about 1 g to about 10 g, about 10 g to about 100 g, or about 50 g to about 200 g. In an aspect, the amount of the composition administered is, about 0.01 mL, about 0.05 mL, about 0.1 mL, about 0.5 mL, about 1 mL, about 2 mL, about 5 mL, about 10 mL, about 20 mL, about 30 mL, about 40 mL, about 50 mL, about 60 mL, about 70 g, about 80 mL, about 90 mL, about 100 mL, or about 150 mL. In an aspect, the amount of the composition administered is about 0.01 mL to about 0.1 mL, about 0.1 mL to about 1 mL, about 1 mL to about 10 mL, about 10 mL to about 100 mL, or about 50 mL to about 150 mL.

A composition of the disclosure can be administered to the intended treatment site as a single administration, about hourly, about every 4 hours, about every 6 hours, about every 12 hours, about daily, about every two days, about weekly, about biweekly, about monthly, about bimonthly, about every 6 months, about annually or about biannually. In an aspect, the composition of the disclosure can be re-administered once the effect of the initial administration is deemed to be essentially complete. In an aspect, the composition of the disclosure can be re-administered once the effect of the initial administration is deemed to be about 80% complete.

In an aspect, the composition of the disclosure can undergo swelling upon use, upon reconstitution or upon application onto or into a tissue. In an aspect, the composition of the disclosure can swell by greater that 5000% as measured by the final swollen weigh divided by the initial weight. In an aspect, the composition of the disclosure can swell by about 2500% to 5000%. In an aspect, the composition of the disclosure can swell by about 1000% to 2500%. In an aspect, the composition of the disclosure can swell by about 500% to 1000%. In an aspect, the composition of the disclosure can swell by about 100% to 500%. In an aspect, the composition of the disclosure can swell by about 50% to 100%. In an aspect, the composition of the disclosure can swell by about 25% to 50%. In an aspect, the composition of the disclosure can swell by about 10% to 25%. In an aspect, the composition of the disclosure can swell by about 0.1% to 10%. In an aspect, the composition of the disclosure can swell by less that about 10%. In an aspect, the composition of the disclosure can swell by less that about 5%. In an aspect, the composition of the disclosure does not swell.

In an aspect, the derivatized hyaluronic acid is a mercaptobenzoic acid derivatized hyaluronic acid. In an aspect, the derivatized hyaluronic acid is a 2-mercaptobenzoic acid derivatized hyaluronic acid. In an aspect, the derivatized hyaluronic acid is a thiophenol derivatized hyaluronic acid. In an aspect, the crosslinker is a 1,4-butanediol diglycidyl ether (BDDE).

In an aspect, compositions as disclosed herein are formulated for, and are useful for viscosupplementation. The compositions may or may not crosslinked, and compositions may optionally contain a biologically active agent.

Viscosupplementation is the process of injecting a composition into the joint to relieve pain. In an aspect, a composition used is based on hyaluronic acid or a derivative thereof such as one or more of compositions disclosed herein. A composition can be injected into the various joint spaces of the body. Suitable joints include knee, shoulder, ankle, elbow, hip, trapeziometacarpal Joint, finger joint, wrist joints, temporomandibular joint, back and neck. In an aspect, the compositions used can further comprise crosslinks. The compositions can further comprise one or more excipient. The compositions of the disclosure that can be used for osteoarthritis treatment can be injected through a needle of between 18 gauge and 21 gauge. The compositions of the disclosure can further comprise a biologically active agent. In an aspect, the biologically active agent can be a corticosteroid, a local anesthetic, an antibody, a peptide or an anti-inflammatory. The volume of the solution that comprises a composition of the disclosure can range from 0.5 ml to 10 mL with an aspect being in the 2 mL to 6 mL for injection into the knee. In an aspect, the crosslinked hydrogel particles are suspended in a saline solution. In an aspect, the hydrogel particles are suspended in a solution of hyaluronic acid or a hyaluronic acid derivative on this disclosure. In an aspect, the crosslinked hydrogel suspension is in a prefilled syringe in which the contents of the syringe are sterile.

In an aspect, the compositions and compositions as disclosed herein are formulated for, and are useful for, adhesion prevention. The compositions may or may not crosslinked, and compositions may optionally contain a biologically active agent. Areas of the body where adhesion prevention is wanted include spinal, abdominal, coating on dura substitute, nasal, ear, elbow, and tendon. Exemplary biologically active agents include anti-inflammatory and pain medicines.

In an aspect, compositions of this disclosure may be used to reduce the incidence and severity of adhesions and scar tissue that may occur following injury or a surgical procedure. These adhesions can include abdominal adhesions, pelvic adhesions, heart adhesions, joint adhesions, tendon adhesions (e.g. flexor tendon, Achilles tendon, patella tendon), spinal adhesions, lumbar adhesions, nerve adhesions, dural adhesions, sinus adhesions. Compositions can further comprise one or more excipient. Compositions of the disclosure can further comprise a biologically active agent. In an aspect, the biologically active agent can be a corticosteroid, a local anesthetic, an antibody, a peptide or an anti-inflammatory. In an aspect, a composition of this disclosure comprises hyaluronic acid or a hyaluronic acid derivative. In an aspect, the composition can be in the form of a crosslinked hydrogel. In an aspect, a composition of the disclosure can be in a crosslinked form that has been lyophilized to form a porous foam or it could be as a solid or perforated film.

In an aspect, compositions as disclosed herein are formulated for, and are useful for, tissue sealing. Compositions may or may not crosslinked, and compositions may optionally contain a biologically active agent.

In an aspect, a composition of the disclosure that contains residual vinyl sulfone groups can be reacted with a compound that has 2 or more free thiol functional groups such that a crosslinked composition is produced. In an aspect, a composition of the disclosure that contains free vinyl sulfone groups can be prepared as a solution. In an aspect, the solution can be prepared using saline. In an aspect, a composition of the disclosure that contains residual vinyl sulfone groups can be prepared as a first solution and a composition that has 2 or more free thiol functional groups can be prepared as a second solution. The pH of either the first or the second solution can be adjusted such than pH of the solution is greater than pH 8. This can be accomplished by using a solution that has a pH of greater than 8 to dissolve either a composition of the disclosure that contains residual vinyl sulfone groups or the compound that has 2 or more free thiol functional groups, adding buffer components to either a composition of the disclosure that contains residual vinyl sulfone groups or to the compound that has 2 or more free thiol functional groups.

In an aspect, the first and second solution can be combined to form mixture composition and applied to the tissue surface. In an aspect, the mixture composition can be applied through a needle or cannula. In an aspect, the mixture composition can be applied using a spray applicator. Examples of spray applicators include but are not limited to the Fibrijet SA-3674 and SA-3675. In an aspect, the mixture composition can be applied using a gas assisted spray applicator. Examples of gas assisted spray applicators include but are not limited to the Fibrijet SA-3651 and SA-3652.

In an aspect, a composition can be applied to the tissue in a liquid form and after 3 minutes a composition is in a gel form. The time required to convert from the liquid form to the gel form depends on the specific application. In an aspect the liquid to gel conversion can take less than 2 minutes. In an aspect the liquid to gel conversion can take less than 30 seconds. In an aspect, the liquid to gel conversion can take less than 15 seconds.

A composition for tissue sealing may further comprise an excipient.

In an aspect, a composition of the disclosure can be used as an embolic composition to stop or reduce the blood flow to a tissue. In an aspect, the embolic composition can be used in trans-arterial embolization (TAE), trans-arterial chemoembolization (TACE), drug-eluting bead chemoembolization (DEB-TACE) or a combination thereof. In an aspect, the embolic composition can be used for tumor embolization. In an aspect, the tumors that can be treated include but are not limited tojuvenile nasopharyngeal angiofibroma (JNA), hemangiopericytoma, Glomus jugulare and other paragangliomas, metastatic lesions, meningiomas, hemangioblastomal, hepatic tumors, brain tumors, or any hypervascular tumor. In an aspect, the embolic composition can be used for uterine fibroid embolization (UFE), pelvic embolization, ovarian vein embolization, ovarian artery embolization, and arteriovenous malformations (AVM). In an aspect, the composition can be a preformed hydrogel. In an aspect, the preformed hydrogel can be in the form of a particle. In an aspect, the particle can be approximately spherical in shape. In an aspect, the surface of the sphere can be smooth. In an aspect, the spheres can be fully hydrated such that they don't increase in size by greater than about 10% when delivered to the desired embolization site. In an aspect, the spheres can have a size of about 30 m to about 1500 μm in the hydrated state. In an aspect, the spheres can have a size of about 40 m to about 120 μm, 100 μm to about 300 μm, about 300 to about 500 μm, about 500 μm to about 700 μm, about 700 um to about 900 μm, about 900 μm to about 1200 μm or a combination thereof. In an aspect, the preformed hydrogel can be in the form of a rod. The rod can be placed within a catheter. The catheter can be inserted into either an artery or a vein and the tip of the catheter can be maneuvered to desired portion of the vessel that needs to be embolized. The rod can be expelled from the catheter such that it occludes the target vessel. In an aspect, the rod can be in a form that once expelled from the catheter, the rod increases in cross sectional area. In an aspect, the rod can be in a dry form in the catheter or it can further comprise a water-soluble solvent. A water-soluble solvent can include but is not limited to ethanol, isopropanol, polyethylene glycol, methoxy polyethylene glycol, dimethyl formamide (DMF), n-methylpyrollidone (NMP) or a combination thereof. The rod can comprise a surfactant or emulsifying agent. In an aspect, the rod can be in a hypertonic environment in the catheter such that once expelled into the physiological environment, the rod increases in cross-sectional area.

A composition can further comprise a biologically active agent. In an aspect, the biologically active agent can be a chemotherapeutic agent. In an aspect, the biologically active agent includes but is not limited to doxorubicin, paclitaxel, epirubicin, irinotecan, cisplatin, mitomycin C or combinations thereof.

In an aspect, compositions of this disclosure are combined with a biologically active agent to treat bacterial vaginosis. Compositions of this disclosure can be formulated such than compositions are tissue adhesive and adheres to the vaginal tissue for a period of greater than 2 hours. Compositions can further comprise one or more excipient. Compositions of the disclosure can further comprise a biologically active agent. In an aspect, the biologically active agent can be an antibacterial agent. In an aspect, the antibacterial can be selected from the group consisting of clindamycin, tinidazole, metronidazole, secnidazole and ornidazole. The formulations comprising compositions of this disclosure, can be applied intravaginally.

In an aspect, compositions of the disclosure are selected to provide ocular application. For example, eye drops for dry eyes/lubricating eye drops for contact lenses.

In an aspect, compositions of this disclosure can be used as eye drops. The eye drops can be used to treat dry eyes, punctate epitheliopathies, a disease of the eye, infected ocular tissue, inflamed ocular tissue, as a lubricant for the surface of the eye, as a lubricant for use with or without contact lenses and to assist in healing of the eye following trauma or a surgical procedure to the eye or surrounding tissue. Surgical procedures to the eye can include but are not limited to cataract surgery, intra-ocular lens replacement, fixing a detached retina, tumor removal, glaucoma surgery, photorefractive keratectomy, refractive surgery, corneal surgery, vitreo-retinal surgery, eye muscle surgery, oculoplastic surgery, surgery involving the lacrimal punctum, canaliculus, and sac. An ocular formulation comprising compositions of this disclosure can further comprise an excipient. Compositions of this disclosure can be formulated into a solution or suspension with is then administered to the eye. An ocular formulation comprising one or more compositions of this disclosure can further comprise a biologically active agent. The biologically active agent can be present as part of the solution or it can be in the form of a suspension or emulsion. Compositions of this disclosure can be formulated into a solution or suspension with is then administered to the eye.

In an aspect, compositions of this disclosure can be prepared to be used to lubricate and wet contact lenses. The contact lens can be immersed prior to use or could be stored in a solution that contains the composition of this disclosure. The solution can comprise one or more excipients. The solution can further comprise boric acid or sodium borate. The solution can be formulated to be preservative free.

In an aspect, a composition of this disclosure can be formed into a formulation that is inserted into the lacrimal punctum, the lacrimal canaliculus or the lacrimal sac. A composition of the disclosure can be in the form of a solution, swollen hydrogel or a dehydrated hydrogel. In an aspect, a composition can further comprise an excipient. In an aspect, a composition is crosslinked. In an aspect, a composition further comprises a biologically active agent. In an aspect, the biologically active agent can be but is not limited to a corticosteroid (for example, dexamethasone, triamcinolone acetonide, triamcinolone hexacetonide, triamcinolone acetate, betamethasone, fluoromethalone, hydrocortisone, medrysone or prednisolone), prostaglandins (for example, latanoprost, travoprost or bimatoprost), beta blockers (for example timolol or betaxolol), alpha-adrenergic agonists (for example apraclonidine or brimonidine), carbonic anhydrase inhibitors (for example dorzolamide or brinzolamide), mitotic agents, chlorinergic agents (for example pilocarpine), anti-bacterial or antifungal agents. Anti-bacterial agents include but are not limited to moxifloxacin, besifloxacin, tobramycin, gentamicin, ofloxacin, levofloxacin, azithromycin, gatifloxacin, ciprofloxacin, erythromycin, bacitracin, and trimethoprim-Polymyxin B.

In an aspect, a composition is crosslinked in the presence of the biologically active agent and then dried. In an aspect, a composition is crosslinked, dried, reswollen in the presence of a biologically active agent and then dried. The dried formulation can be of suitable dimensions such that it can be placed in the lacrimal punctum. Upon contact with lachrymal fluid and tears, the dried formulation hydrates, and swells in such a manner as to be physically retained in the punctum. In an aspect, the dried formulation can be inserted into the canaliculus. Upon contact with lachrymal fluid and tears, the dried formulation hydrates, and swells in such a manner as to be physically retained in the canaliculus. The formulation releases the contained biologically active agent over a period of 24 hours to 3 weeks. In an aspect, the biologically active agent is released in a sustained manner for a period of 7 days. In an aspect, the biologically active agent is released in a sustained manner for a period of 4 weeks. In an aspect, the dried formulation can be inserted intravitreally so than biologically active agent can be delivered into the vitreous of the eye. In an aspect, the dried formulation could be inserted into the anterior chamber of the eye.

In an aspect, compositions of the disclosure are selected to provide a punctal plug. The punctal plus may incorporate a biologically active agent, e.g., steroid or a pain relief drug.

In an aspect, a composition of this disclosure can be used to treat mucositis. Examples of mucositis include oral and vaginal mucositis. During cancer treatments, the rapidly divided epithelial cells lining the gastro-intestinal tract (which goes from the mouth to the anus) break down leaving the mucosal tissue open to ulceration and infection. This leads to mucositis. Oral mucositis can often occur following chemotherapy and radiation treatments. It can lead pain and increased risk of infection. This can lead to nutritional problem due to these symptoms reducing the ability and desire to eat. Providing a coating that covers these lesions, can reduce the pain and potential for infection. A composition of this disclosure can be formulated such that the composition is tissue adhesive and adheres to the mucosal tissue of the mouth tissue or the vagina for a period of greater than 2 hours. A composition can further comprise one or more excipients. A composition of the disclosure can further comprise a biologically active agent. In an aspect, the biologically active agent can be a local anesthetic, an anti-infective, an anti-inflammatory or a combination thereof. Local anesthetics can include but are not limited to benzocaine, bupivacaine, amethocaine, lignocaine, lidocaine, cocaine, cinchocaine, dibucaine, mepivacaine, prilocaine, etidocaine, veratridine (specific c-fiber blocker) and procaine. For the oral mucositis, the compositions of the disclosure can be formulated such that it can be applied as an oral rinse or applied as a gel. For vaginal mucositis, a composition of the disclosure can be formulated such that it can be applied intravaginally to the vaginal tissue surface.

In an aspect, a composition of this disclosure can be used to treat a surgical site during and following canalplasty, tympanoplasty, myringoplasty, stapedectomy mastoid procedures, or any other procedure related to the ear. A composition can be used to modulate wound healing as well as to control bleeding. A composition of this disclosure can be in the form of a lyophilized sponge, an electrospun matrix, a film, a gel or a combination of these forms. A composition of the disclosure can comprise an excipient. In an aspect, a composition of the disclosure can comprise a biologically active agent.

In an aspect, a composition of the disclosure can be used to treat otitis media, acute otitis externa, balance disorders (for example Meniere' disease), tinnitus and sensorineural hearing loss. A composition of this disclosure can be in the form of a solution, a suspension, a lyophilized sponge, an electrospun matrix, a film, a gel, a solid rod-like form, or a combination of these forms. A composition of the disclosure can comprise an excipient. In an aspect, a composition of the disclosure can comprise a biologically active agent. To treat infections of the ear, a composition can comprise an antibiotic, an antibacterial, an antiviral, an antifungal or a combination thereof. In an aspect, a composition can comprise amoxicillin, clavulanate, cefuroxime axetil, ceftriaxone, Levofloxacin, a cephalosporin, a trimethoprim-sulfamethoxazole, a macrolide, Ofloxacin, Gentamicin sulfate, Tobramycin sulfate and ciproflaxin, In an aspect, a composition can comprise a corticosteroid. Corticosteroids can include but are not limited to betamethasone, betamethasone valerate, cortisone, dexamethasone, dexamethasone 21-phosphate, dexamethasone sodium phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone, triamcinolone acetonide. In an aspect, a combination of an antibiotic and a corticosteroid can be added to a composition if the disclosure. In an aspect, a composition of the disclosure can be applied to the area to be treated by being applied with a dropper, a syringe, through a needle or catheter or by physically placing a composition.

In an aspect, a composition of the disclosure can be used to treat lumbar radicular pain and sciatica. One or more non-crosslinked compositions of the disclosure can be formed into an aqueous solution that comprises an anti-inflammatory, growth factor or anesthetic agent. In an aspect, the administered composition does not comprise any particulate composition greater than 0.2 μm. In an aspect, the agent is a water-soluble corticosteroid. In an aspect, the agent is dexamethasone sodium phosphate. In an aspect, a composition of the disclosure is about 0.2% (w/w) to about 2% (w/w) of the administered composition. In an aspect, a composition of the disclosure is about 0.5% (w/w) to about 1.5% (w/w) of the administered composition. In an aspect, the composition is administered as a transforaminal epidural injection or a lumbar epidural injection.

In an aspect, a composition of the disclosure can comprise a biologically active agent. The compositions of this disclosure can be used as a matrix from which the biologically active agent can be delivered. In an aspect the release profile of the biologically active agent into a phosphate buffered saline solution if slower than that of the normal dissolution profile of the biologically active agent. In an aspect, a composition of the disclosure can be in the form of a crosslinked gel.

In an aspect, the treatment using the drug delivery formulation can be a single injection or could be two or more injections that are separated by a period of time. The composition can be injected subcutaneously, intra-dermally or intra-muscularly. The composition can be injected through a needle, trocar, catheter, tube, or cannula.

In an aspect, the contents of the prefilled syringe or vial are sterile. In an aspect, the contents of the prefilled syringe or vial are stable at 2-8° C. or 20-25° C. for at least 6 months, preferably 12 months and most preferably 24 months. In an aspect, the drug delivery formulation can be applied topically or by instillation.

In an aspect, compositions of this disclosure in a crosslinked form can be used as device to plug a defect following the removal of a piece of tissue or the needle track following a biopsy procedure. In an aspect, a crosslinked form of a composition can be prepared and then dried. The dried composition can be delivered into the needle track or the site that a piece of tissue was removed. The dried composition can absorb moisture from the surrounding tissue to rehydrate and swell such than swollen size is larger than the initial size of a composition. The swollen composition is then retained at the site into which it is placed. In an aspect, the crosslinked dried composition can be used to seal a hole in the tissue where the crosslinked composition is placed in the hole and it swells to seal off the hole. An example of this could be to seal lung tissue following puncturing of the lung following a biopsy or surgical procedure. In an aspect, the crosslinked dried composition can comprise a metal piece that is visible under x-ray or fluoroscopy examination. The metal piece can take on various forms such as but not limited to a flat piece, a rod, a coil, a loop, a hoop, hook, a number and a letter of the alphabet. In an aspect, the crosslinked dried composition can comprise a biologically active agent. In an aspect, the biologically active agent can have hemostatic properties. In an aspect, the crosslinked dried composition can comprise collagen, chitosan or thrombin.

In an aspect, compositions of the present disclosure are formulated for, and are useful for, a plug for female sterilization. Female sterilization can be accomplished by inserting a plug into the fallopian tube. This plug can provide a physical barrier to the passage of the ovum into the uterus as well as to the sperm reaching the ovum. The predominant procedure to effect female sterilization in a laparoscopic procedure in which the fallopian tubes are severed and then ligated. In other versions of the procedure, the fallopian tubes can be closed using clips or rings to clamp them closed. Cauterization has also been used to seal the fallopian tubes. These procedures are generally classed as major surgery, usually requires general anesthesia and the patient requires a recuperation period. Transvaginal sterilization procedure was an alternative to the laproscopic procedures as they were less invasive. Initial transvaginal procedures used chemical agents such as Sodium morrhuate, Quinacrine. Methyl cyanoacrylate and silver nitrate but the success rates and side effects have limited their use. Hysteroscopic tubal sterilization has emerged as a minimally invasive alternative to conventional tubal ligation. Hysteroscopic tubal sterilization can be performed in approximately 10 minutes in an office setting without the use of general or even local anesthesia.

Two hysteroscopic tubal sterilization products were commercialized. The Essure system consists of a device insert that is loaded into a single-use delivery system. The device consists of an inner coil of stainless steel and polyethylene terephthalate (PET) fibers and an outer coil of nickel-titanium (nitinol). The metal components hold the device in place while the PET fibers allow tissue ingrowth into the device which will lead to occlusion of the fallopian tube. This ingrowth process does take time and so the patient must use other forms of contraception for 3 months. At this stage, a hysterosalpingogram is performed to confirm placement and tubal occlusion. The device is permanent and remains in the patient for the rest of the patient life. This product was removed from the marketplace because of safety concerns. A study concluded that patients undergoing hysteroscopic sterilization using the Essure device have a similar risk of unintended pregnancy but a more than 10-fold higher risk of undergoing reoperation compared with patients undergoing laparoscopic sterilization (BMJ 2015;351:h5162).

Another sterilization method was developed by Hologic. The Adiana® sterilization method used radiofrequency energy to cause controlled thermal damage of the lining of the fallopian tube lumen. Followingthe thermal injuryto the fallopian tube, a porous non-degradable silicone plug is placed in the thermally injured fallopian tube. Over a few weeks, tissue ingrowth into the porous plug results in occlusion of the fallopian tube. A hysterosalpingogram is performed at 3 months to confirm tubal occlusion. The silicone plug is a permanent implant. The Adiana® system has been withdrawn from the market.

The Essure system and the Adiana® system both left a permanent device in the patient. Having a system that comprises a degradable plug component would be beneficial in that no composition will remain permanently within the patient. The method and devices described herein provide a means to occlude the fallopian tube that will result in a reduction in the ability of a female to become pregnant. The method involves mechanically injuring the lining of the fallopian tube followed by the insertion of a degradable plug.

The method for mechanically injuring the fallopian tube is to insert a device that comprises a rough surface into the fallopian tube and then physically move the device in a rotational motion, a linear motion that follows the fallopian tube or a combination thereof. This motion can be repeated more than once. This physical movement is continued until the endothelial layer of the fallopian tube where the physical motion occurs is either partially removed or completely removed.

The device used to denude the endothelial layer of the fallopian tube can comprise a series of fibers radiating from a central core. In an aspect this device is similar in structure to a bottle brush.

In an aspect, the fibers can be spaced evenly apart in a continuous manner. In an aspect, the fibers can be in rows with spaces between the rows. In an aspect, the fibers could be oriented in a spiral shape along the axis of the device. In an aspect, the fibers can be oriented in one or more linear rows that are aligned about parallel with the axis from which they emanate. In an aspect, the fibers are in one or more rows such than rows are about perpendicular to the axis from which they emanate.

In an aspect, the fibers can be made from a nondegradable polymer. The polymers that can be used to prepare the fibers include but are not limited to polyethylene, polypropylene, polyethylene terephthalate (PET), nylon, polyurethane, polyetheretherketone (PEEK), polyaryletherketone (PAEK), fluorocarbon polymers such as polytetrafluoroethylene, silk and combinations thereof.

In an aspect, the fibers can be made from a metal. The metals that can be used to prepare the fibers include but are not limited to stainless steel, titanium, nitinol, magnesium, alloys of Co—Cr—Mo, Cr—Ni—Cr—Mo, CP—Ti, Ti—Al—V, Ti—Al—Nb, Ti-13Nb-13Zr, Ti—Mo—Zr—Fe or combinations thereof.

In an aspect, the central core of the denuding device can comprise a core prepared from the twisting of 2 or more metal strands together such than fibers are trapped between the twisted metal strands. The metals that can be used to prepare the central core include but are not limited to stainless steel, titanium, nitinol, magnesium, alloys of Co—Cr—Mo, Cr—Ni—Cr—Mo, CP—Ti, Ti—Al—V, Ti—Al—Nb, Ti-13Nb-13Zr, Ti—Mo—Zr—Fe or combinations thereof.

In an aspect, the terminal end of the central core that is first introduced into the fallopian tube can comprise an atraumatic tip that does not damage the tissue as the device is being guided into the desired location in the fallopian tube. This atraumatic tip can be a rounded end cap, a domed shaped end, a cone shaped end with a rounded tip. The surface of the atraumatic tip can have a smooth surface. The atraumatic tip can be made of a non-degradable polymer or a metal. The non-degradable polymers that can be used to manufacture the atraumatic tip include but are not limited to polyethylene, polypropylene, polyethylene terephthalate (PET), nylon, polyurethane, polyetheretherketone (PEEK), polyaryletherketone (PAEK), fluorocarbon polymers such as polytetrafluoroethylene, silk and combinations thereof. The metals that can be used to prepare the atraumatic tip include but are not limited to stainless steel, titanium, nitinol, magnesium, alloys of Co—Cr—Mo, Cr—Ni—Cr—Mo, CP—Ti, Ti—Al—V, Ti—Al—Nb, Ti-13Nb-13Zr, Ti—Mo—Zr—Fe or combinations thereof.

The atraumatic tip can be attached to the central core by a crimping process, a molding process, a process that uses an adhesive to bond the tip to the central core, or a thermal process.

The plug can comprise a hydrogel. In an aspect, the hydrogel is prepared using the crosslinked composition of this disclosure. A hydrogel in the form a rod that is larger than the size of the fallopian tube is prepared. The hydrogel rod is then dried. The hydrogel can be dried at normal atmospheric pressures or under reduced atmospheric pressure. In an aspect, the hydrogel can be lyophilized. Once delivered to the desired site, the hydrogel plug would absorb moisture from the fallopian tube and swell. The swelling of the hydrogel plug will enable the hydrogel plug to be retained at the site where it was placed.

In an aspect, the hydrogel can further comprise a porogen to facilitate the formation of pores within the hydrogel. The porogen can comprise particulates. The particulates can comprise a degradable polymer. Degradable polymers that can be used as porogens include but are not limited to degradable polyesters, polyanhydrides, polyurethanes, polyether-esters, polycarbonates, polyether-carbonates, polyether-ester carbonates, polkyhydroxyalkanoates, polyamides and polymers that are synthesized from one or more monomers from the group of l-lactide, dl-lactide, glycolide, ε-caprolactone, trimethylene carbonate, morpholine-dione, p-dioxanone and 1,5-dioxapan-2-one.

In an aspect, the porogen can be leeched out of the hydrogel during the device manufacturing process. This can be accomplished by incubating the porogen containing hydrogen in a solvent in which the porogen will dissolve. The solvent is preferably a water miscible solvent. In an aspect, the porogen can remain in the device throughout the manufacturing process and will degrade and leech out once the hydrogel plug is inserted into the patient.

In an aspect the plug comprises a degradable polymer. Degradable polymers that can be used in the plug include but are not limited to degradable polyesters, polyanhydrides, polyurethanes, polyether-esters, polycarbonates, polyether-carbonates, polyether-ester carbonates, polkyhydroxyalkanoates, polyamides and polymers that are synthesized from one or more monomers from the group of l-lactide, dl-lactide, glycolide, ε-caprolactone, trimethylene carbonate, morpholine-dione, p-dioxanone and 1,5-dioxapan-2-one.

The plug can comprise a monofilament structure, a multifilament structure, or a braided structure. In an aspect, the plug can be prepared by taking particles or chopped fibers of the degradable polymer and compression mold them into a shape. Heat can be used to thermally fuse the particulates together such that a porous structure is obtained. In an aspect, the shape can be in the form of a rod. The porous rod can then be cut to a predetermined length.

In an aspect, the plug can be made from an electrospun degradable polymer. In an aspect, the plug is made from a thin film of electrospun composition. The plug can be cut directly from a sheet of the electrospun composition. In an aspect, the plug can be prepared by rolling an electrospun film into a roll. The electrospun plug or the rolled rod shaped structure can be coated with a second degradable polymer such that the rolled configuration is retained. In an aspect, the polymer used to prepare the rolled structure has a degradation time that is longer than the polymer used to coat the rolled structure. This can allow the plug to be more rigid which makes handling easier during manufacturing but upon delivery to the desired site, the faster degrading composition will start degrading and facilitate tissue ingrowth while the first longer lasting polymer provides a scaffold for the ingrowing tissue.

In an aspect, the electrospun plug can be coated or dipped into a solution of a water-soluble polymer. The plug is then dried at ambient pressure or at reduced pressure. The plug may also be dried by lyophilization. The presence of the water soluble polymer can make the electrospun composition more rigid and thus easier to handle during manufacturing and delivery to the intended site. Once positioned at the intended site, the polymer will start to dissolve and leech out of the electrospun composition. The tissue from the mechanically damaged fallopian tube can then grow into the electrospun composition. The electrospun composition will degrade over time leaving an occluded fallopian tube. In an aspect, the water soluble polymer can be selected from the group of polyethylene oxide, polyethylene glycol, block copolymers of polyethylene glycol and polypropylene glycol (e.g. pluronics F126 and pluronics F68), dextran, hyaluronic acid, or a hyaluronic acid derivative of this disclosure.

The degradable polymer used to form the plug can further comprise a porogen. The porogen can comprises an inorganic salt, an organic small molecule or a polymer. The porogen is selected such that it is soluble in a solvent in which the biodegradable polymer used to prepare the plug has limited solubility.

Inorganic salts that can be used as porogens include but not limited to sodium salts, potassium salts, calcium salts, magnesium salts, aluminum salts, copper salts, barium salts, iron salts. Examples of these salts include but are not limited to sodium chloride, sodium bromide, sodium iodide, sodium sulfate, sodium phosphate, sodium hydrogen phosphate, or combinations thereof.

A porous plug can be prepared by 3D-printing the plug. A degradable polymer can be used to 3D print the plug. In an aspect, the degradable polymer that can be used in the plug include but are not limited to degradable polyesters, polyanhydrides, polyurethanes, polyether-esters, polycarbonates, polyether-carbonates, polyether-ester carbonates, polkyhydroxyalkanoates, polyamides and polymers that are synthesized from one or more monomers from the group of l-lactide, dl-lactide, glycolide, ε-caprolactone, trimethylene carbonate, morpholine-dione, p-dioxanone and 1,5-dioxapan-2-one.

The plug can comprise position retaining features. These features can include non-symmetrical shapes, barbs, ridges, pores, slits, slots, or a combination thereof. The barbs can be unidirectional in that they all point in the same direction or the barbs could point in two or more different directions. The barbs could be uniformly spaced on the plug or they could be present in only specific portions of the plug.

In an aspect, the plug can be dipped into a solution of a composition of the disclosure. The solution can then be activated to allow the solution to crosslink such than pores of the plug comprise the crosslinked composition. The crosslinking process can be activated by adjusting pH of the solution, addition of a crosslinked, elevation of temperature, addition of an initiator or a combination of one or more of these.

In an aspect, a composition of the disclosure can be used as a scaffold to allow the ingrowth of tissue or bone. In an aspect, a composition of this disclosure can be prepared as a crosslinked matrix that is then lyophilized. The lyophilized composition can then be rehydrated in the presence of cells such than hydrated matrix acts as a scaffold that allows the growth of the cells on and into the scaffold. In an aspect, a composition of this disclosure that have residual vinyl sulfone groups, can be electrospun to form a porous matrix. The electospun fibers can then be crosslinked using heat, ultraviolet, e-beam or gamma radiation. In an aspect, a composition of the disclosure that contains residual vinyl sulfone groups can further comprise a photocrosslinker. A solution of this composition can be electrospun and then the electrospun matrix can be subjected to ultraviolet radiation such than photocrosslinker results in crosslinking of a composition. The resultant matrix can be rehydrated in the presence of cells such that it acts as a scaffold for tissue growth. In an aspect, carboxylic acid containing compositions of this disclosure can be electospun into a matrix by mixing a solution of a composition of this disclosure with a solution of a multivalent cation just prior to electrospinning. In an aspect, a solution of a carboxylic acid containing composition of this disclosure could be placed in one syringe and a solution of a multivalent cation or a cationic polymer can be placed in another syringe. The syringes can be connected via a y-connector and a needle can be connected to final arm of the y-connector. The two solutions can then be pumped through the needle and this mixture can be electrospun onto a surface such that the composition of the disclosure is ionically crosslinked. Multivalent cations can include calcium, magnesium, ferric ions, ferrous ions, zinc, aluminum and chromium.

Cationic polymers that can be used include but are not limited to chitosan and derivatives thereof, polyvinyl pyrollidone, peptides containing more than one lysine group and polyethyleneimine.

In an aspect, a solution of a composition of this disclosure can be used to coat a degradable or non-degradable scaffold matrix. In an aspect, a composition of this disclosure that has been modified with alkyl or aryl groups can be used to coat a scaffold for tissue growth. The alkyl or aryl groups will interact with the scaffold through hydrophobic bond while the hydrophilic portion of a composition will allow for cell growth on the coated scaffold surface. In an aspect, composition compositions of this disclosure that have residual vinyl sulfone groups, can be coated onto the scaffold. The coated scaffold can be subjected to heat which will result in a composition transforming into a crosslinked composition.

In an aspect, compositions of the disclosure can comprise a sulfonate group. In an aspect, compositions of the disclosure can comprise both hydrophobic groups and sulfonate groups. The hydrophobic groups can be alkyl or aromatic based.

In an aspect, tissue scaffold support structure can be 3D printed or electrospun using a degradable polymer. The degradable polymer that can be used can include but not limited to degradable polyesters, polyanhydrides, polyurethanes, polyether-esters, polycarbonates, polyether-carbonates, polyether-ester carbonates, polkyhydroxyalkanoates, polyamides and polymers that are synthesized from one or more monomers from the group of l-lactide, dl-lactide, glycolide, ε-caprolactone, trimethylene carbonate, morpholine-dione, p-dioxanone and 1,5-dioxapan-2-one.

In an aspect the polymer used to 3D print or electrospin the scaffold can further comprise an inorganic filler or a combination of inorganic fillers. In an aspect the inorganic filler can be selected from the group calcium carbonate, calcium phosphate, tricalcium phosphate, hydroxyapatite, bioglass, or a combination thereof.

In an aspect, 3D-printed or electrospun scaffold can be coated with a solution of one or more compositions of the disclosure. This composition can be coated onto the scaffold through a dip coating or spray coating process. In an aspect, the coated scaffold can be dried. The drying process can include drying at elevated temperature, drying at reduced pressure or lyophilization. In an aspect, the solution of one or more compositions of the disclosure can further comprise a biologically active agent.

In an aspect, a scaffold can be dipped into a solution of one or more compositions of the disclosure. The solution can then be activated to allow the solution to crosslink such that the pores of the scaffold comprise the crosslinked composition. The crosslinking process can be activated by adjusting pH of the solution, addition of a crosslinked, elevation of temperature, addition of an initiator or a combination of one or more of these.

In an aspect, scaffold can be dipped into a solution of one or more compositions of the disclosure and a crosslinking agent. The rate of the crosslinking reaction can be controlled such that the scaffold can be coated with a composition prior to complete crosslinking of the composition. In an aspect a biologically active agent can be incorporated into a composition before or immediately following the initiation of the crosslinking reaction. The scaffold can then be coated with this composition and once applied to the scaffold, the crosslinking reaction is completed such that the device comprises the crosslinked composition with the biologically active agent essentially encapsulated by the crosslinked composition.

In an aspect, a composition of this disclosure used to prepare a scaffold or to coat the scaffold can comprise a biologically active agent. In an aspect, the biologically active agent can enhance cell growth. In an aspect, the biologically active agent can be one or more growth factors or peptides that enhance cell growth and cell adhesion. In an aspect, a composition of this disclosure used to prepare a scaffold or to coat the scaffold can further comprise an excipient. In an aspect, a composition of the disclosure can comprise one or more extracellular matrix components. The extracellular matrix component can include but are not limited to heparan sulfate, chondroitin sulfate, keratin sulfate, hyaluronic acid, collagen, elastin, fibronectin, and laminin.

In an aspect, the cells that can be added to the scaffolds that contain a composition of this disclosure include embryonic stem cells, mesenchymal stem cells, adipose-derived stem cell, endothelial stem cells, dental pulp stem cells, tumor cells, chondrocytes, osteoblasts, dermal fibroblasts, hepatocytes, smooth muscle cells, endothelial cells, epithelial cells and cardiac cells

In an aspect, compositions of the present disclosure comprise free vinyl sulfone functional groups and can be used to 3D print structures. The compositions can be prepared as solutions with viscosities that allow them to be 3D printed. In an aspect, a solution of one or more compositions with residual vinyl sulfone groups can be prepared. A second solution containing a composition with at least two free thiol groups can be prepared. In an aspect, the first and second solution can be mixed together. Just prior to printing, the pH of the mixture can be adjusted to a pH of greater than 8, preferably greater than 9, such that the mixture can be printed and then cure following printing. In an aspect, the pH can be adjusted by mixing the mixture with a buffer solution that has a pH of greater than 8. The mixing takes place just prior to the print head ensuring that the mixture does not gel up in the print head and thus clot the printer, In an aspect, the solution of a composition that comprises the residual vinyl sulfone functional groups can has its pH adjusted to a pH of greater than 8 by mixing it with a buffer solution. This solution can then be mixed with solution 2 just prior to the print head such that the mixture is printed and then allowed to complete gelation once printed.

The viscosity of the mixture can be used to control the retention of the printed structure until gelation is completed. In an aspect, a thermogelling composition can be added to either the first, second or buffer solution. Thus, the mixture can be printed and then the temperature of the printed environment can be different from the solution prior to printing such that following the printing process the printed solution undergoes thermal gelation to preserve the initial printed structure while the crosslinking process is moving towards completion.

Thermogelling compositions can include but are not limited to polyethylene-block-polypropylene co polymers such as Pluronics F127 or F68 or polyester-polyethylene glycol block co polymers. The polyester-polyethylene glycol copolymers can include deblock and triblock copolymers. The polyester component are polymers that are synthesized from at least one of the monomers from the group of l-lactide, dl-lactide, glycolide, ε-caprolactone, morpholine-dione, p-dioxanone and 1,5-dioxapan-2-one. In an aspect, a thermogelling polymer that comprises trimethylene carbonate can be used.

Following the completion of the gelation process, the printed construct can be rinsed to neutralize the pH of the printed gel. In an aspect the printed structure can be dried such than residual water content is less than 10%. In an aspect, the printed structure can be lyophilized.

The printed structure can be used as a tissue scaffold, for wound healing applications, for occlusion of a lumen, a biopsy site or a needle tract.

For procedures such as neuroendoscopy, intracranial decompression, and treatment of chronic subdural hematoma, holes are often drilled into the skull. These are often referred to as burr holes. In many instances, these burr holes are left untreated following the surgical procedure and the scalp is replaced directly over these holes. This can lead to scalp depressions at the burr hole. These scalp depressions can lack mechanical strength. In order to prevent this, a burr hole plug can be inserted into the burr hole such that it can facilitate and support bone regrowth. Autologous bone can be used to fill the burr holes but this requires harvesting of the bone. Synthetic compositions can be used as burr hole plugs. A degradable burr hole plug that degrades while facilitating bone ingrowth will allow the healing of the burr hole without leaving residual composition. A polycaprolactone (PCL) burr hole plug has been commercialized. The challenge with PCL is that it is slow degrading and the interface between the polymer and the in-growing tissue is usually not the best due to the hydrophobicity of the polymer.

A composition of the disclosure can be made into a burr hole plug. A solution of a composition can be placed in the mold and then the composition can be lyophilized to produce a porous structure that can be inserted into the burr hole. In an aspect, a composition of the disclosure can be electrospun and then cut to form a plug that can be inserted into the burr hole. In an aspect, a solution of a composition of the disclosure can be placed in a mold and the solution can be crosslinked. The crosslinked plug can be used directly. In an aspect, the crosslinked composition can be lyophilized to yield a porous crosslinked structure that can be used as a burr hole plug.

In an aspect, a burr hole plug can be 3D printed or electrospun using a degradable polymer. The degradable polymer that can be used can include but not limited to degradable polyesters, polyanhydrides, polyurethanes, polyether-esters, polycarbonates, polyether-carbonates, polyether-ester carbonates, polkyhydroxyalkanoates, polyamides and polymers that are synthesized from one or more monomers from the group of l-lactide, dl-lactide, glycolide, ε-caprolactone, trimethylene carbonate, morpholine-dione, p-dioxanone and 1,5-dioxapan-2-one.

In an aspect the polymer used to 3D print or electrospin the burr hole plug can further comprise an inorganic filler or a combination of inorganic fillers. In an aspect the inorganic filler can be selected from the group calcium carbonate, calcium phosphate, tricalcium phosphate and hydroxyapatite.

In an aspect, the 3d-printed or electrospun burr plug can further comprise an extracellular matrix composition. In an aspect, the extracellular matrix composition can be selected from the group collagen, hyaluronic acid, chondroitin sulfate, heparan sulfate, keratin sulfate, elastin, fibronectin and laminin.

In an aspect, 3D-printed or electrospun plug can be coated with a solution of a compositions of the disclosure. This composition can be coated onto the plug through a dip coating or spray coating process. In an aspect, the coated plug can be dried. The drying process can include drying at elevated temperature, drying at reduced pressure or lyophilization.

In an aspect, polymeric degradable plug can be dipped into a solution of a compositions of the disclosure. The solution can then be activated to allow the solution to crosslink such that the pores of the plug comprise the crosslinked composition. The crosslinking process can be activated by adjusting pH of the solution, addition of a crosslinker, elevation of temperature, addition of an initiator or a combination of one or more of these.

In an aspect, polymeric degradable plug can be dipped into a solution of one or more compositions of the disclosure that contain residual vinyl sulfone groups. The coated device can be dried at elevated temperatures to remove the solvent and to allow crosslinking of the coating such that the pores of the plug comprise the crosslinked composition.

In an aspect, crosslinked forms of compositions of this disclosure can be used to form nerve guides. Optionally, the nerve guides can be prepared by lyophilization. In an aspect, collagen, gelatin, chitosan heparan sulfate or a combination of these can be further added to a composition of the disclosure to form the nerve guides. In an aspect, Schwann cells can be incorporated into a composition during the formation of the nerve guide.

In an aspect, compositions if this disclosure can be prepared as a solution that has a viscosity of greater than 50 cP. In an aspect, this solution can be applied to tissue to reduce the coefficient of friction with the tissue surface. In an aspect, composition can be used as a vaginal lubricant. In an aspect, the solution can be applied to a device that is to be inserted into an opening, orifice or cavity such that the solution act to lubricate the passage of the device through the opening, orifice or cavity. In an aspect the device could be an endoscope.

In an aspect, a composition of this disclosure can be used to coat a medical device. Medical devices that can be coated include but are not limited to a catheter, a needle, a biopsy needle, a tissue marker, a guide wire, and endoluminal sheath, a suture, a braid, a trocar, a hernia mesh, a surgical mesh, a contact lens, an intra-ocular lens, a stent (for example vascular stent, esophageal stent, biliary stent coronary stent, renal stent, peripheral vascular stent), a nasal splint, a vascular graft, a stent-graft, aneurysm coils, introducer sheaths, balloon catheters, vascular closure devices, inferior vena cava filter, and Hydrocephalic shunts.

In an aspect, a kit comprises a composition of this disclosure. In an aspect, the kit can comprise a composition of this disclosure and a syringe. In an aspect, the kit can comprise a composition of this disclosure and a syringe wherein the contents of the syringe are sterile. In an aspect, the kit can further comprise a needle. In an aspect, the needle can be a 16G, 17G, 18G, 19G, 20G, 21G, 22G, 23G, 24G, 25G, 26G, 27G or a 30G needle. In an aspect, the kit can further comprise an outer package that houses the other components of the kit. In an aspect, the outer packaging can comprise a thermoformed polymer. In an aspect, a kit can further describe written instructions for use of the one or more compositions in the kit.

In an aspect, a composition of the disclosure can be prepared as a solution which can then be applied by spray coating or dip coating. The solvent can then be removed to leave a coating of a composition of the disclosure on the device surface. In an aspect, the solution can be an aqueous solution. In an aspect, the solution can comprise an organic solvent. In an aspect, the solution can comprise water and a water-miscible organic solvent. In an aspect a composition of the disclosure can be functionalized with aliphatic or aromatic groups such that there is a hydrophobic interaction with these groups and the device surface. In an aspect, a composition of this disclosure that has residual vinyl sulfone groups can be coated onto a medical device by dip coating or spray coating. The coating is dried. The coating can be exposed to heat, gamma, e-beam or ultraviolet radiation to crosslink a composition. In an aspect, the coating can further comprise a biologically active agent. In an aspect, the coating when hydrated, increase the lubricity of the coated device. The increased lubricity of the coated device can be measure by a decrease of the water contact angle by at least 20°. In an aspect, the increased lubricity can be measured as a decrease in the friction coefficient by at least 20%. In an aspect, the device can be partially coated with some part of the device remaining uncoated. In an aspect, the device can be precoated with binding polymer coating that enhances the binding of the coating composition of this disclosure. In an aspect, the coating can further comprise heparin, to give the coating anti-thrombotic properties.

The following Examples are offered by way of illustration and not by way of limitation. In the Examples, DI stands for distilled water or deionized water, PEG stands for polyethylene glycol and IV stands for intrinsic viscosity.

### EXAMPLES

### Example 1 DVS Modified HA (DVS1)

2.5 g sodium hyaluronate (900 KDa) was added to a glass 4 L reaction kettle. The lid, overhead stirrer and anchor impellor were attached to the reaction kettle. The solution was then stirred at about 200 rpm. 250 g deionized water was added to the kettle. The solution was stirred for about 18 hrs. 166.5 g of a 0.25 M NaOH solution was added to the dissolved sodium hyaluronate. The pH of the solution was measured after 2 min and was found to be 12.69. A freshly prepared solution of 10.6 g divinyl sulfone in 66 g of DI water was then rapidly added to the stirring solution. After 75 seconds, 50 g of a 1M HCl solution was added to the reaction mixture. 1 M NaOH was then added dropwise until the solution pH was between 5 and 7. 6 g NaCl was then added to the solution. Once the NaCl had dissolved, 1.25 L acetone was slowly added over a period of 20 minutes. The suspension was stirred for about 3 hours. 200 mL denatured ethanol was added and the solution was stirred for about 30 minutes. The precipitate was filtered under vacuum using a sintered glass funnel through a 0.22 m PTFE filter membrane. Once all the solution had been filtered, the vacuum was disconnected and 100 mL ethanol was used to rinse the precipitate. The ethanol was then removed by vacuum filtration. This process was repeated an additional 3 times. The product was dried under vacuum at room temp in a vacuum oven. Approximately 10-20 mg of the dried sample was added to a vial. D₂O was added to the sample to make the final concentration of the solution about 6 mg/mL. The sample was shaken on an orbital shaker until dissolved. Once dissolved, the sample was transferred into a NMR tube and the 1H-NMR spectrum of the sample was recorded on a NMR spectrometer. The spectrum was printed out with the specific peaks in the 6.3-6.5 ppm (2 peaks from the 2 CH₂=protons from the vinyl sulfone residue), the 6.8-7.0 ppm (CH peak of vinyl group) and 1.8-2.5 ppm (singlet from the 3 CH₃ protons from the N-acetyl group of the HA) regions being integrated. The percent modification is calculated on molar ratio of the vinyl CH protons (6.8-7 ppm) to the acetamide (1.8-2.5 ppm) protons. The percent substitution was found to be about 8.9%.

### Example 2 DVS Modified HA (DVS2

1133 g deionized water was added to a 5 L reaction vessel. The overhead stirrer was set at 300 rpm. 11.33 g sodium hyaluronate [HA] (approx. 800 kDa; approx. 1.4 m³/Kg IV) was added to a 5 L reaction kettle. A heating tape was placed around the 5 L kettle and the temperature was set to 25° C. The solution was allowed to stir until the HA has dissolved. 50 g divinyl sulfone [DVS] was added to 282 g deionized water and the solution was stirred for about 15 minutes. The overhead stirrer was then set at about 750 rpm, 35 g 1M NaOH was added and the pH of the HA solution was adjusted to about 12.3 using NaOH and HCl. The DVS solution was added rapidly and the reaction was allowed to proceed for 10 minutes. During the reaction the pH was maintained at about 12.3 using 1N NaOH. 1M HCl was added to adjust the solution pH to about pH 6 to 8. About 20 g NaCl was added and stirring continued until the NaCl was dissolved. 2 L acetone was added slowly to the reaction mixture. The reaction mixture was stirred for 3 hrs, then about 400 mL ethanol was added and the solution was stirred for 30 min. The precipitated composition was filtered and then washed with four aliquots of 200 mL ethanol. The DVS modified HA was dried under vacuum. The percent substitution, as determined by the procedure described in Example 1, was found to be about 25%. The reaction was repeated using a reaction time of 20 minutes. The percent substitution, as determined by the procedure described in Example 1, was found to be about 50%.

### Example 3 DVS Modified HA (DVS3

1533 g deionized water was added to a 5 L reaction vessel. The overhead stirrer was set at 300 rpm. 11.33 g sodium hyaluronate [HA] (approx. 800 kDa; approx. 1.4 m³/Kg IV) was added to a 5 L reaction kettle. A heating tape was placed around the 5 L kettle and the temperature was set to 25° C. The solution was allowed to stir until the HA had dissolved. 50 g divinyl sulfone [DVS] was added to 282 g deionized water and the solution was stirred for about 15 minutes. About 15 g NaCl was added to the HA solution. Once dissolved, the overhead stirrer was then set at about 750 rpm and about 75 g 1N NaOH was added to the solution. The pH of the HA solution was adjusted to about 12.3 using NaOH and HCl. The DVS solution was then added rapidly and the reaction was allowed to proceed for 20 minutes. During the reaction the pH was maintained at about 12.3 using 1N NaOH. 1M HCl was added to adjust the solution pH to about pH 6 to 8. About 20 g NaCl was added and stirring continued until the NaCl was dissolved. 2 L acetone was added slowly to the reaction mixture. The reaction mixture was stirred for 3 hrs, then about 400 mL ethanol was added and the solution was stirred for 30 min. The precipitated composition was filtered and then washed with four aliquots of 200 mL ethanol. A sample of the DVS modified HA was dried under vacuum and analyzed by NMR. The percent substitution, as determined by the procedure described in Example 1, was found to be about 80%.

### Example 4 MBA Modified Hyaluronic Acid

The DVS-derivatized HA samples prepared in Examples 3 and 4 were reacted separately with 2-mercaptobenzoic acid (MBA). The DVS-derivatized HA was added to a 5 L reaction vessel. About 660 g deionized water was added. The overhead stirrer was set to about 300 rpm and the system was heated to about 30° C. About 425 g ethanol was added to the reaction mixture. The reaction mixture was stirred for 18 hrs. The stirring speed was increased to about 500 rpm and about 15.3 g 2-mercaptobenzoic acid was added to the reaction mixture. The pH of the reaction mixture was adjusted to about pH 9.0 using 1M NaOH and 1M HCl. The reaction was allowed to run for about 16-18 hours. The pH was then adjusted to about pH6.7 to 7.3 and the heating was turned off. About 4.7 g NaCl was then added to the reaction mixture. Once the NaCl was dissolved about 2 L acetone was added to the mixture. The mixture was stirred for about 90 minutes. The stirrer was turned off and the settled precipitate was filtered. The precipitate was washed 4 times with 200 mL ethanol. The product was dried under vacuum. A sample of the product was dissolved in D₂O and the ¹H-NMR spectrum was measured. The presence of MBA substitution was evidenced by peaks at 7.1-7.5 ppm (Ar—H). The MBA molar substitution, as calculated from the integrals at 7.1-7.5 ppm (Ar—H) and 1.7-2 ppm (HA-acetamide), was found to be about 25%, 50% and 80% respectively.

### Example 5 Solution Rheology of Hyaluronic Acid Derivative

Hyaluronic acid derivative (as prepared according to Examples 2, 3 and 4) solutions were made by dissolution at a 2% w/v in deionized water. Samples were allowed to dissolve on a roller overnight. Rheological measurements were performed using TA Discovery HR-2 rheometer with 20 mm 2° measuring system at 25° C. Frequency oscillation measurements were performed where a variable frequency sweep from 0.1 to 10 Hz was applied to the sample in logarithmic increments. The strain percentage was determined by running an amplitude oscillation test. All samples had a soak time of 60 seconds prior to testing.

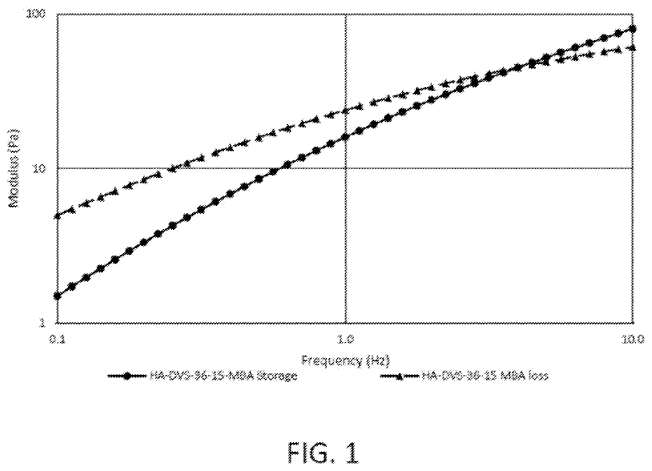

FIG. 1 shows the storage (G′) and loss (G″) modulus for a 2-mercaptobenzoic acid hyaluronic acid derivative (HA-DVS-MBA) of the disclosure. The storage and loss modulus at various frequencies are shown in Table 1. At low frequencies the storage modulus (G′) is less than the loss modulus (G″) while at higher frequencies, the storage modulus (G′) is greater than the loss modulus (G″). This shows than hyaluronic acid derivative is not crosslinked.

TABLE 1

| | 0.25 Hz | | 0.50 Hz | | 8.9 Hz | | 10.0 Hz | | % |
|---|---|---|---|---|---|---|---|---|---|
| Sample | G′ | G″ | G′ | G″ | G′ | G″ | G′ | G″ | Substitution |
| 36-13 | 15.7 | 19.9 | 23.9 | 27.4 | 111.3 | 76.5 | 117.4 | 78.8 | 51.4 |
| 36-15 | 4.3 | 10.0 | 8.6 | 15.9 | 74.8 | 59.1 | 80.2 | 61.2 | 80.6 |
| 36-16 | 4.4 | 14.8 | 10.2 | 24.5 | 105.7 | 91.9 | 112.6 | 94.8 | 10.5 |
| 36-17 | 3.5 | 8.0 | 6.5 | 12.5 | 51.2 | 49.5 | 54.5 | 51.5 | 83.3 |
| 36-18 | 4.2 | 13.2 | 9.2 | 21.5 | 86.9 | 78.4 | 92.4 | 80.9 | 51.0 |
| 36-19 | 7.9 | 13.3 | 13.3 | 19.4 | 74.2 | 60.9 | 77.9 | 62.8 | 85.2 |
| 36-20 | 4.9 | 10.4 | 9.5 | 16.2 | 73.8 | 58.4 | 78.6 | 60.5 | 84.3 |
| 36-21 | 6.2 | 12.2 | 11.7 | 19.0 | 88.2 | 66.8 | 93.8 | 69.1 | 83.4 |

### Example 6 Synthesis of Divinyl Sulfone Derivatized HA-Different Molecular Weights

The synthesis of divinyl sulfone derivatized HA using different starting HA molecular weights was performed using a similar method as described in Example 1. The specific molecular weights, reaction conditions and vinyl sulfone substitution obtained are as set forth below in Table 2.

TABLE 2

| | Rxn1 | Rxn1 | Rxn3 |
|---|---|---|---|
| Approx. Mw (kD) | 2,300 | 200 | 510 |
| HA (g) | 2.5 | 5 | 5 |
| Water (g) | 500 | 500 | 500 |
| DVS (g) | 10.6 | 21.2 | 21.2 |
| DVS water (g) | 66 | 132 | 132 |
| Stir speed (rpm) | 200-250 | 200-250 | 200-250 |
| Reaction pH | >12.5 | >12.5 | >12.5 |
| Reaction time (min) | 1.25 | 1.25 | 1.25 |
| NaCl (g) | 4 | 12 | 12 |
| Acetone (mL) | 2000 | 2000 | 1750 |
| Ethanol (mL) | 200 | 400 | 400 |
| Ethanol wash (mL) | 100 | 200 | 200 |
| Substitution (%) | 8.1 | 6.4 | 9.3 |

### Example 7 Synthesis of Hyaluronic Acid Derivatives

The about 50% substituted DVS-HA derivative (made in a similar manner as Example 2) was reacted in separate experiments with an excess of thiophenol and 1-sodium-3-mercapto-1-propanesulfonate compounds using similar experimental conditions as described in example 4. The substitution levels were 49% and 54% for the thiophenol and 1-sodium-3-mercapto-1-propanesulfonate respectively.

### Example 8 Solution Degradation of Hyaluronic Acid Derivatives

A solution degradation study was performed using 1% (w/v) solution of the HA derivative in PBS and 0.3 IU/mL hyaluronidase. The viscosity of the solutions were measured at various time points using a TA Discovery HR-2 rheometer with 20 mm 2° measuring system at 25° C. The percent change in viscosity was calculated by taking the difference in viscosity at a timepoint as compared to the initial viscosity and dividing by the initial viscosity. The rate of viscosity change was measured by the difference in viscosity at a measured time to that of the initial viscosity all over the time in hours. The derivatized HA compounds has a slower degradation rate than the unmodified HA at 1, 2, 4 and 6 hours as measured by percent viscosity change, shown in Table 3, and rate of viscosity change, shown in Table 4 (also shown in FIG. 4).

TABLE 3

| | Percent | Viscosity change (%) | | | | |
|---|---|---|---|---|---|---|
| Sample | substitution | 0 h | 1 h | 2 h | 4 h | 6 h |
| HA | N/A | 0 | −21.6 | −36.7 | −54.5 | −64.1 |
| HA-DVS-MBA | 82 | 0 | −14.1 | −25.3 | −37.3 | −45.3 |
| HA-DVS-MBA | 51 | 0 | −18.7 | −28.8 | −40.0 | −50.6 |
| HA-DVS-Thio | 49 | 0 | −17.3 | −24.5 | −40.7 | −48.8 |
| HA-DVS-SMPS | 54 | 0 | −19.4 | −31.6 | −46.7 | −54.7 |

### Example 9 Gel Preparation and Hyaluronidase Degradation

TABLE 4

Rate of Viscosity Change (Pa/h)

| Sample | 1 h | 2 h | 4 h | 6 h |
|---|---|---|---|---|
| HA | −190 | −161 | −120 | −94 |
| HA-DVS-MBA-82 | −57 | −51 | −38 | −30 |
| HA-DVS-MBA-51 | −85 | −66 | −46 | −39 |
| HA-DVS-Thio-49 | −104 | −74 | −62 | −49 |
| HA-DVS-SMPS-54 | −101 | −83 | −61 | −48 |

HA gels were prepared by dissolving HA (10% w/v) in a 0.25M NaOH solution (pH about 13.1). The sample was vortexed and placed on a roller mill until dissolved. 200 uL BDDE was added to the solution. After mixing, about 1 g of the solution was added to a well of a 24 well cell culture plate. The samples were placed in an oven at about 50° C. for about 4 hours. The gelled samples were removed from the wells and each gel was placed in about 50 mL water. After 1 hr, the water was decanted and replaced with fresh water. After 1 hr, the water was decanted and replaced with 50 mL phosphate buffered saline. The gels were incubated overnight. HA-DVS-MBA gels were made in a similar manner except the 0.25% NaOH solution comprised about 5% v/v) ethanol. The gels were weighed and then about 1.5 g of each gel was incubated in 30 mL 2 IU/mL hyaluronidase (bovine testes). The samples were incubated for a specific period of time at about 37° C. The mass of the gel was measured after each timepoint. The hyaluronidase solution was replaced with fresh solution. The change in gel mass were calculated. The percent weight change of the gels at various time points are shown in table 5. Since the gels swelled slightly during the first six hours incubation, the weight changes were also normalized to the 6 hour time point. The weight changes (percent) normalized to the 6 h time point are shown in table 6. The HA-DVS-MBA gel degradation was slower than that of HA (FIG. 5). Tables 5 and 6 show that the HA-DVS-MBA gels degrade more slowly than the unmodified HA gel.

TABLE 5

| | % | Percent weight change | | | | |
|---|---|---|---|---|---|---|
| Material | Substitution | 0 h | 6 | 24 | 48 | 72 |
| HA | N/A | 0 | 3.3 | −14.1 | −37.9 | −59.9 |
| 36-15 | 80.6 | 0 | 10.2 | 6.3 | −8.6 | −34.6 |
| 37-7 | 33.9 | 0 | 9.5 | −1.5 | −24 | −51.7 |

TABLE 6

Percent weight change
(normalized to 6 hrs)

| Material | % Substitution | 0 h | 18 h | 42 h | 66 h |
|---|---|---|---|---|---|
| HA | N/A | 0 | −16.8 | −39.8 | −61.1 |
| 36-15 | 80.6 | 0 | −3.6 | −17.1 | −40.7 |
| 37-7 | 33.9 | 0 | −10.1 | −30.7 | −55.9 |

### Example 10-Gel Formation as a Function of pH

Crosslinked HA-DVS-MBA gels were prepared with crosslinking at different pHs. The HA-DVS-MBA had a substitution of 83.3%. The HA-DVS-MBA was dissolved in an aqueous solution of different pH that contained about 5% (v/v) ethanol and about 1% (v/v) BDDE. The samples were mixed and then placed in an oven at about 50° C. for about 3 hours. The gelled samples were washed with about 200 mL PBS for about 1 hr. This process was repeated 2 times. The gels were washed overnight in 400 mL PBS and then for an additional hour with 200 mL PBS. About 1.5 g of each of the gels weighed and then each gel was incubated in 30 mL 2 IU/mL hyaluronidase (bovine testes). The samples were incubated for a specific period of time at about 37° C. The mass of the gel was measured after each timepoint. The hyaluronidase solution was replaced with fresh solution. The change in gel mass was calculated. Since the gels swelled during the first six hours incubation, the weight loss changes were also normalized to the 6 hour time point. The data showed than degradation profile can be tuned by adjusting the pH at which the HA-DVS-MBA/BDDE composition is crosslinked. Table 7 shows the percent swelling and the concentration of the gels. The percent swelling was calculated by subtracting the initial gel weight from the final gel weight and then dividing the result by the initial gel weight. The concentration was calculated by dividing the mass of HA-based material used to make the gel by the final gel weight of the gel. Table 8 shows the percent weight change and the percent weight change normalized to 6 h as a function of time. Table 7 shows that by adjusting the pH of the crosslinking reaction, the percent swelling and concentration if the HA-based material in the gel can be altered. Table 8 shows that the by decreasing the altering the pH of the crosslinking reaction, the degradation profile can be altered. By decreasing the pH of the crosslinking reaction from 13.1 to 12.4, the degradation rates of the gels were reduced.

TABLE 7

| | | | | | | Concen- |
|---|---|---|---|---|---|---|
| | | Polymer | Initial Gel | Final Gel | % | tration |
| Gel | pH | Mass (g) | Mass (g) | Mass (g) | Swelling | (mg/mL) |
| 1 | 13.1 | 0.502 | 3.7196 | 17.3702 | 367 | 28.9 |
| 2 | 12.9 | 0.5037 | 3.5593 | 12.6984 | 257 | 39.67 |
| 3 | 12.6 | 0.5015 | 3.8048 | 9.6974 | 155 | 51.71 |
| 4 | 12.4 | 0.5002 | 3.8404 | 8.6387 | 125 | 57.9 |

TABLE 8

| | | | | | | | Weight change (%) | | | |
|---|---|---|---|---|---|---|---|---|---|---|
| | | Weight change (%) | | | | | relative to 6 h | | | |
| Gel | pH | 0 h | 6 h | 24 h | 53 h | 96 h | 0 h | 18 h | 47 h | 90 h |
| 1 | 13.1 | 0 | 44.2 | 27.1 | −27.8 | −91.3 | 0 | −11.8 | −50 | −94 |
| 2 | 12.9 | 0 | 24.2 | 27.7 | 13.3 | −6.4 | 0 | 2.9 | −8.7 | −24.6 |
| 3 | 12.6 | 0 | 9.5 | 12.1 | 12.4 | 8.5 | 0 | 2.4 | 2.7 | −0.9 |
| 4 | 12.4 | 0 | 7.7 | 8.2 | 8.7 | 8.3 | 0 | 0.5 | 0.9 | 0.5 |

### Example 11 Gel Formation as a Function of pH

The crosslinked gels were prepared in a similar manner as those in example 10 using HA-DVS-MBA (84.3% substitution). Hyaluronidase degradation was performed similar to that described in Example 10. Table 9 shows the percent swelling and the concentration of the gels as calculated according to example 10. This show that by modulating the pH of the crosslinking reaction, the percent swelling and concentration of the HA-based material can be modulated. As the pH of the crosslinking in increased from pH 11.2 to pH 12.4, the percent swelling decreases and the concentration of the HA-based material in the gel increases. Table 10 shows the percent weight change and the percent weight change normalized to 6 h of the prepared gels as a function of time. This shows that the degradation profile of the gel can be modulated by adjusting the pH of the crosslinking reaction. Increasing the pH of the crosslinking reaction from pH 11.2 to pH 12.4 results in gels that have slower degradation profiles.

TABLE 9

| | Reaction | Polymer | Initial Gel | Final Gel | % | Conc. |
|---|---|---|---|---|---|---|
| Gel | pH | Mass (g) | Mass (g) | Mass (g) | Swell | (mg/mL) |
| A | 12.4 | 0.5003 | 3.7195 | 8.4627 | 128 | 59.12 |
| B | 11.9 | 0.5005 | 3.7683 | 9.2677 | 146 | 54 |
| C | 11.6 | 0.5002 | 3.787 | 12.2336 | 223 | 40.89 |
| D | 11.2 | 0.4995 | 3.7691 | 24.0399 | 538 | 20.78 |

TABLE 10

| | | | | | | Weight change (%) | | |
|---|---|---|---|---|---|---|---|---|
| | Reaction | Weight change (%) | | | | relative to 6 h | | |
| Gel | pH | 0 h | 6 h | 24 h | 48 h | 0 h | 18 h | 44 h |
| A | 12.4 | 0 | 3.3 | 3.5 | 5.3 | 0 | 0.2 | 2.0 |
| B | 11.9 | 0 | 12.0 | 10.1 | 10.7 | 0 | −1.7 | −1.1 |
| C | 11.6 | 0 | 16.4 | 12.6 | 11.7 | 0 | −3.3 | −4.1 |
| D | 11.2 | 0 | 27.3 | −17.7 | −84.2 | 0 | −35.4 | −87.6 |

### Example 12 Rheology and Injectability of Gels

A portion of the gel samples (example 11) were mixed with unmodified HA (20 mg/mL) in various ratios. The samples were then autoclaved at 121° C. for 5 minutes. The rheology of the gels and the diluted gels were performed using a TA Discovery HR-2 rheometer with 20 mm 2° measuring system at 25° C. Frequency oscillation measurements were performed where a variable frequency sweep from 0.1 to 10 Hz was applied to the sample in logarithmic increments. The strain percentage was determined by running an amplitude oscillation test. The injection forces were measured using a load cell in an MTS and an extrusion rate of 12 mm/minute. The samples were filled into a 1 mL syringe, a 27 G was attached to the syringe and then sample was expelled from the syringe at 12 mm/minute using the MTS. The process was repeated using a 30 G needle. Table 11 shows that the storage modulus (G′), loss modulus (G″), and viscosity of the gels and the gels diluted with unmodified HA can be modulated by altering the pH at which the crosslinking reaction is performed. Table 12 shows that the maximum injection force and the average injection force required to pass the gels through a 27 G or 30 G needle can be altered by adjusting the pH at which the crosslinking reaction is performed.

TABLE 11

| | Gel Material | Gel:HA | G′ (Pa) @ | | G″ (Pa) @ | | tan | |
|---|---|---|---|---|---|---|---|---|
| Sample | (example 11) | (w/w) | 1 Hz | st dev | 1 Hz | st dev | delta | Viscosity |
| 1 | A | 100:0 | 12655.9 | 797.2 | 865.6 | 207.0 | 0.068 | 2019143 |
| 2 | A | 75:25 | 6797.9 | 597.7 | 788.4 | 79.5 | 0.116 | 1089174 |
| 3 | B | 100:0 | 9530.7 | 424.2 | 1058.2 | 37.3 | 0.111 | 1526190 |
| 4 | B | 75:25 | 5709.5 | 1073.7 | 956.0 | 122.4 | 0.167 | 921391 |
| 5 | C | 100:0 | 3042.6 | 341.3 | 294.2 | 56.0 | 0.097 | 486508 |
| 6 | C | 75:25 | 2396.2 | 95.7 | 522.4 | 18.5 | 0.218 | 390351 |
| 7 | D | 100:0 | 23.6 | 0.4 | 22.6 | 9.6 | 0.958 | 5271 |
| 8 | D | 75:25 | 23.9 | 0.7 | 23.5 | 0.8 | 0.984 | 5338 |

TABLE 12

| | Sample | | | | | | | |
|---|---|---|---|---|---|---|---|---|
| | 2 | | 4 | | 6 | | 8 | |
| Needle Gauge | 27 | 30 | 27 | 30 | 27 | 30 | 27 | 30 |
| Max Force (N) | 49.2 | 59.7 | 46.6 | 79.2 | 47.8 | 81.2 | 15.8 | 33.5 |
| Average force | 31.8 | 42.6 | 28.7 | 52.0 | 27.7 | 48.5 | 4.6 | 12.7 |
| (after 5 mm) | | | | | | | | |
| [N] | | | | | | | | |
| Average SD | 4.0 | 3.7 | 4.5 | 9.3 | 6.6 | 14.7 | 1.6 | 4.9 |

### Example 13 Gel Preparation

The crosslinked gels were prepared in a similar manner as those in example 10 using HA-DVS-MBA (85.2% substitution). A portion of the gel samples were passed about 20 time from one syringe to another to break up the gel. The samples were autoclaved for 5 minutes at 121° C. The samples were mixed with unmodified HA (20 mg/mL) in various ratios. The rheology of the gels was performed using a TA Discovery HR-2 rheometer with 20 mm 2° measuring system at 25° C. Frequency oscillation measurements were performed where a variable frequency sweep from 0.1 to 10 Hz was applied to the sample in logarithmic increments. The strain percentage was determined by running an amplitude oscillation test. Tables 13 shows that by modulating the initial concentration of the HA-based material used and the pH of the crosslinking reaction, the percent swelling and the concentration of the HA-based material in the final gel can be modulated. Table 14 shows that the storage modulus (G′), loss modulus (G″), and viscosity of the gels and the gels diluted with unmodified HA can be modulated by altering the concentration of the starting HA-based material and the pH at which the crosslinking reaction is performed.

TABLE 13

| | | | Crosslinking | | | | |
|---|---|---|---|---|---|---|---|
| | Crosslinking | Polymer | Solution | Initial Gel | Final Gel | % | Conc. |
| Sample | pH | Mass (g) | Mass (g) | Mass (g) | Mass (g) | Swell | (mg/mL) |
| A | 12.4 | 0.2508 | 3.4016 | 3.5333 | 11.2941 | 220 | 22.21 |
| 1B | 12.4 | 0.2517 | 3.4044 | 3.4541 | 10.3248 | 199 | 24.38 |
| 2A | 12.9 | 0.2525 | 3.3996 | 3.2875 | 19.4535 | 492 | 12.98 |
| 2B | 12.9 | 0.252 | 3.4035 | 3.1975 | 17.7118 | 454 | 14.23 |
| 3A | 12.6 | 0.2503 | 3.3944 | 3.4841 | 10.5178 | 202 | 23.8 |
| 3B | 12.6 | 0.2506 | 3.4015 | 3.4511 | 12.4646 | 261 | 20.1 |
| 4A | 12.6 | 0.3503 | 3.4074 | 3.6339 | 11.4746 | 216 | 30.53 |
| 4B | 12.6 | 0.3501 | 3.3959 | 3.6587 | 11.7577 | 221 | 29.78 |

TABLE 14

| Gel | Gel:HA | G′ (Pa) | STD | G″ (Pa) | STD | tan | Complex |
|---|---|---|---|---|---|---|---|
| Material | (w/w) | @ 1 Hz | dev | @ 1 Hz | dev | delta | Viscosity |
| 1B | 100:0 | 1179.73 | 15.24 | 130.26 | 2.76 | 0.11 | 188902 |
| 1B | 75:25 | 397 | 27 | 129 | 1 | 0.32 | 66373 |
| 1B | 60:40 | 334 | 43 | 132 | 3 | 0.39 | 57229 |
| 2B | 100:0 | 1.05 | 0.12 | 1.23 | 0.08 | 1.17 | 258 |
| 3A | 100:0 | 425 | 8 | 81 | 3 | 0.19 | 68838 |
| 3A | 75:25 | 208 | 9 | 73 | 1 | 0.35 | 35107 |
| 4A | 100:0 | 2113 | 73 | 252 | 15 | 0.12 | 338628 |
| 4A | 75:25 | 1055 | 37 | 256 | 7 | 0.24 | 172739 |

### Example 14 Gel Formation with Different BDDE Levels

Crosslinked gels were prepared using differing levels of BDDE. The crosslinked gels were prepared in a similar manner as those in example 10 using HA-DVS-MBA (85.2% substitution) and HA-DVS-MBA (84.3% substitution). Hyaluronidase degradation was performed similar to that described in Example 10. Tables 15 and 16 shows that by altering the amount of BDDE used in the crosslinking reaction, the percent swelling, concentration of the HA-based material in the gel and the degradation profile can be modulated. The HA-DVS-MBA crosslinked samples had a slower degradation profile that the HA crosslinked gel.

TABLE 15

| | | | Polymer | % | Conc. | Weight Change (%) | | | |
|---|---|---|---|---|---|---|---|---|---|
| Material | pH | % BDD | Mass (g) | Swelling | (mg/mL) | 0 h | 6 h | 24 h | 48 h |
| HA | 13.1 | 1 | 0.5002 | 541 | 20.33 | 0 | 38.2 | 0.8 | −60.5 |
| HA-DVS-MBA (85.2) | 12.4 | 1 | 0.5004 | 126 | 57.95 | 0 | 7.6 | 10.7 | 10.8 |
| HA-DVS-MBA (85.2) | 12.4 | 0.5 | 0.5002 | 156 | 54.82 | 0 | 8.7 | 13.0 | 8.2 |
| HA-DVS-MBA (85.2) | 12.4 | 0.25 | 0.5005 | 201 | 49.87 | 0 | 8.8 | 12.1 | 9.1 |

TABLE 16

| | | | Mass (g) | % | Conc. | Weight Change (%) | | | |
|---|---|---|---|---|---|---|---|---|---|
| Material | pH | % BDDE | Polymer | Swelling | (mg/mL) | 0 h | 6 h | 24 h | 48 h |
| HA-DVS-MBA (84.3) | 12.4 | 0.05 | 0.5001 | 185 | 46.37 | 0 | 23 | 26 | 20 |
| HA-DVS-MBA (84.3) | 12.4 | 0.1 | 0.5005 | 184 | 46.27 | 0 | 10 | 13 | 9 |
| HA-DVS-MBA (84.3) | 12.4 | 0.15 | 0.5005 | 180 | 47.07 | 0 | 11 | 11 | 11 |
| HA-DVS-MBA (84.3) | 12.4 | 0.2 | 0.4994 | 171 | 48.22 | 0 | 12 | 13 | 13 |
| HA | 13.1 | 1 | 0.5 | 592 | 18.97 | 0 | 56 | 30 | −36 |

### Example 15 Gel Formation with Different BDDE Levels

A 1% (v/v) 1,4-butanediol diglycidyl ether (BDDE) solution was prepared by adding 200 uL BDDE to 19.85 mL 1% (m/v) NaOH solution. About 1 mL ethanol is added to the BDDE solution. About 0.5 g of the HA-DVS-MBA derivative (83.3% substitution) was added to a 20 mL glass scintillation vial. 3.4 g of the 1% BDDE/NaOH solution was added to the composition. The vial was capped and vortexed and then mixed with a spatula. The capped sample was placed in an oven at about 50° C. for 3 hours. The crosslinked gel was removed and added to 200 mL PBS. The solution pH was then adjusted to pH 7.0 using 2% (v/v) HCl. After stirring for 1 hr, this washing process was repeated 4 times. The PBS was decanted and the swollen gel mass was measured. The gel was fragmented into smaller pieces and these were added to a 10 mL syringe. An empty 10 mL syringe was connected to the gel containing syringe using a female-female luer-lok connector. The gel was passed back and forth between syringes about 20 times. This process is repeated until all the gel is processed. A portion of the gel is autoclaved at 121° C. for 5 minutes. The gel formation process was repeated using 0.75%, 1.5%, 2%, 2.5% and 3% BDDE. The rheology of the gels were measured using a TA Discovery HR-2 rheometer with 20 mm 2° measuring system at 25° C. The swelling was calculated as the final swollen gel mass/the mass of the initial gel. The concentration was calculated as the mass of HA-derivative used/final mass of swollen gel). Table 17 shows by modulating the amount of BDDE used to crosslinked the HA-DVS-MBA, the storage modulus (G′) and the loss modulus (G″) of the formed gel can be altered.

TABLE 17

| | % | % | Conc. | G′ | G″ | Tan D | G′ | G″ | Tan D |
|---|---|---|---|---|---|---|---|---|---|
| Material | BDDE | Swelling | (mg/mL) | (0.5 Hz) | (0.5 HZ) | (0.5 Hz) | (1 Hz) | (1 HZ) | (1 Hz) |
| HA-DVS-36- | 0.75 | 424 | 25.40 | 210.7 | 56.3 | 0.267 | 230.8 | 51.2 | 0.222 |
| 17-MBA | 1 | 401 | 26.77 | 401.6 | 81.9 | 0.204 | 430.7 | 71.6 | 0.166 |
| | 1.5 | 374 | 27.79 | 555.3 | 105.3 | 0.190 | 592.7 | 95.1 | 0.161 |
| | 2 | 361 | 28.25 | 566.7 | 169.3 | 0.299 | 619.0 | 162.3 | 0.262 |
| | 2.5 | 380 | 27.88 | 725.6 | 190.6 | 0.3 | 796.7 | 186.8 | 0.234 |
| | 3 | 370 | 28.24 | 875.0 | 306.5 | 0.4 | 976.5 | 307.6 | 0.315 |

### Example 16 Crosslinked Gels with Different HA:HA-DVS-MBA Ratios

Crosslinked gels with differing HA to HA-DVS-MBA ratios were prepared. The HA-DVS-MBA had a substitution of 51.4%. The reagents were dissolved in a pH 12.4 aqueous solution that contained 5% (v/v) ethanol. The sample was vortexed and placed on a roller mill until dissolved. 200 uL BDDE was added to each solution. After mixing, about 1 g of the solution was added to a well of a 24 well cell culture plate. The samples were placed in an oven at about 50° C. for about 4 hours. The gelled samples were removed from the wells. The gels from each series were washed with 250 mL 0.005M HCl for 30 min, followed by a DI water wash for 30 min and then a PBS wash for about 15 hrs. The gels (about 2.5 g) were weighed and then each gel was incubated in 30 mL 2 IU/mL hyaluronidase (bovine testes). The samples were incubated for a specific period of time at about 37° C. The mass of the gel was measured after each timepoint. The hyaluronidase solution was replaced with fresh solution. The change in gel mass was calculated. Table 18 shows that the degradation profile can be tuned by adjusting the HA-DVS-MBA: HA ratio and that presence of the HA-DVS-MBA slowed the degradation rate of the gels relative to the HA only gel.

TABLE 18

HA-DVS-MBA:HA ratio (w/w)

| | | | | |
|---|---|---|---|---|
| Timepoints | 100:0 | 75:25 | 50:50 | 0:100 |

| | | | | | |
|---|---|---|---|---|---|
| 0 | h | 0.0 | 0.0 | 0.0 | 0.0 |
| 6 | h | −0.4 | −1.2 | −2.4 | −15.2 |
| 24 | h | −3.7 | −6.8 | −16.3 | −51.4 |
| 48 | h | −8.6 | −15.0 | −38.2 | −84.7 |

### Example 17 Mesh Processed Gels

Gel samples were prepared by adding about 3.4 g of a 0.25M NaOH/5% (v/v) ethanol solution that contained 1% (v/v) BDDE to about 0.5 g HA-DVS-MBA (substitution: 51%) in 20 mL glass scintillation vials. The pH of the final mixture was adjusted to about 12.4. Following mixing, the samples were placed in an oven for about 3 hrs at about 50° C. The samples were washed and equilibrated in PBS (pH 7.4). The gels were processed by adding a 2% (w/v) HA solution to the gels in a 80:20 Gel:HA solution (w/w) and then passing the gels through either a 700 μm mesh, a 300 μm mesh or a 120 μm mesh. The samples were autoclaved at 121° C. for 5 minutes. The storage modulus (G′) and the loss modulus (G″) were measured using a TA Discovery HR-2 rheometer with 20 mm 2° measuring system at 25° C. Table 19 shows the storage (G′) and loss modulus (G″) as a function of particle size.

TABLE 19

| Mesh Size (μm) | G′ (Pa) [1 Hz] | G″ (Pa) [1 Hz] |
|---|---|---|
| 700 | 6660 | 819 |
| 300 | 7495 | 916 |
| 120 | 6046 | 1397 |

### Example 18 Gel Formation without BDDE

About 0.5 g HA-MBA (substitution: 84%) was weighed into a 20 mL glass scintillation vial. A 1% NaOH aqueous solution was prepared. An aliquot of the NaOH solution was removed and the pH was adjusted to a specified pH using HCl. Ethanol was added to make a solution that comprised 5% (v/v) ethanol. The samples were mixed using a spatula and then placed in an oven at about 50° C. for about 3 hours. The gelled samples were washed with about 200 mL PBS (pH about 7.0) for about 1 hr. This process we repeated 2 times. The gels were washed overnight in 400 mL PBS (pH about 7.4) and then for an additional hour with 200 mL PBS (pH about 7.4). A HA control gel was prepared in a similar manner. The HA-DVS-MBA composition formed gels at all the specified pH while the HA composition at pH 13.1 did not form a gel. Table 20 shows that the HA-DVS-MBA composition can be crosslinked without the addition of an externally added crosslinker and that the percent swelling and concentration of the HA-DVS-MBA in the final gel can be modulated by changing the pH at which the self-crosslinking reaction occurs.

TABLE 20

| | | Crosslinking | % | Concentration |
|---|---|---|---|---|
| Vial | Material | pH | Swelling | (mg/mL) |
| A | HA-DVS-MBA | 11.6 | 360 | 29.38 |
| B | HA-DVS-MBA | 12 | 165 | 49.92 |
| C | HA-DVS-MBA | 12.4 | 173 | 47.98 |
| D | HA-DVS-MBA | 12.8 | 310 | 33.07 |
| E | HA-DVS-MBA | 13.1 | 441 | 25.03 |
| F | HA | 13.1 | N/A | N/A |

### Example 19 Rheology of Undiluted and Diluted Gel Samples

A portion of the gel, as prepared in Example 18, was placed in a separate 10 mL syringe. The syringe was connected to a second syringe through a luer connector. The gel was passed back and forth between the syringes a minimum of 30 times. A second portion of the gel samples were mixed with unmodified HA (20 mg/mL) in in a 75:25 w/w ratio. The samples were passed back and forth in a 5 mL syringe. The samples were transferred to a glass scintillation vial and were autoclaved at 121° C. for 5 minutes. The rheology of the gels was performed using a TA Discovery HR-2 rheometer with 20 mm 2° measuring system at 25° C. Frequency oscillation measurements were performed where a variable frequency sweep from 0.1 to 10 Hz was applied to the sample in logarithmic increments. The strain percentage was determined by running an amplitude oscillation test. Table 21 shows that the storage modulus (G′), loss modulus (G″), and viscosity of the gels and the gels diluted with unmodified HA can be modulated by altering the pH at which the self-crosslinking (no external crosslinking agent added) reaction is performed.

TABLE 21

| | | Crosslinking | Undiluted | | Diluted | |
|---|---|---|---|---|---|---|
| Vial | Material | pH | G′ | G″ | G′ | G″ |
| A | HA-DVS-MBA | 11.6 | 532 | 91 | 367 | 119 |
| B | HA-DVS-MBA | 12 | 7632 | 686 | 4540 | 521 |
| C | HA-DVS-MBA | 12.4 | 6979 | 532 | 3681 | 739 |
| D | HA-DVS-MBA | 12.8 | 1268 | 185 | 679 | 176 |
| E | HA-DVS-MBA | 13.1 | 53 | 12 | 45 | 19 |

### Example 20 Degradation of Gels

The gels (about 1.5 g), as prepared in example 18, were weighed and then each gel was incubated in 30 mL PBS solution containing 2 IU/mL hyaluronidase (bovine testes). The samples were incubated for a specific period of time at about 37° C. The mass of the gel was measured after each timepoint. The hyaluronidase solution was replaced with fresh solution. The change in gel mass were calculated. The percent change in gel mass was calculated by dividing the mass of the gel as a specific timepoint by the starting mass of the gel. Since the gels swelled slightly during the first six hours incubation, the weight loss changes were normalized to the 6 hour time point. Table 22 shows that the degradation profile of the self-crosslinking HA-DVS-MBA can be modulated by adjusting the pH at which the self-crosslinking reaction is performed.

TABLE 22

| | | | Change in Mass relative | | | Change in Mass Relative | |
|---|---|---|---|---|---|---|---|
| | | Crosslinking | to T = 0 (%) | | | to T = 6 h (%) | |
| Vial | Material | pH | 6 h | 24 h | 48 h | 18 h | 42 h |
| A | HA-DVS-MBA | 11.6 | 35.6 | 39.0 | 8.4 | 2.6 | −20 |
| B | HA-DVS-MBA | 12 | 16.8 | 14.9 | 10.4 | −5.2 | −5.6 |
| C | HA-DVS-MBA | 12.4 | 12.0 | 10.9 | 13.6 | 2.7 | 1.4 |
| D | HA-DVS-MBA | 12.8 | 38.4 | 52.6 | 38.2 | 10.3 | −0.1 |
| E | HA-DVS-MBA | 13.1 | 78.2 | 50.0 | −32.5 | −5.2 | −62 |

### Example 21 Gel Formation as a Function of Time

13.6 g of a 0.25M NaOH/5% ethanol solution that had the pH adjusted to 12.8, was added to about 2 g of the HA-DVS-MBA (substitution 85.2%). Following mixing, the pH of the solution was adjusted to about pH 12.75. Four aliquots of the composition were transferred to separate 20 mL scintillation vials. The vials were capped and the samples were placed in an oven at 50° C. At 0.5 h, 1 h, 2 h and 3 h, a vial was removed from the oven. The samples were washed and equilibrated overnight in PBS (pH 7.4). The process was repeated using a temperature of 37° C. The swelling and HA-based composition concentration were calculated. Table 23 shows that the percent swelling and self-concentration of the HA-based material can be modulated by adjusting the duration of the crosslinking reaction as well as the temperature at which the self-crosslinking reaction is performed.

TABLE 23

| | | Temp = 50° C. | | Temp = 37° C. | |
|---|---|---|---|---|---|
| | Time | % | Concentration | % | Concentration |
| Sample | (h) | Swelling | (mg/mL) | Swelling | (mg/mL) |
| A | 0.5 | 492 | 22.47 | 412 | 26.33 |
| B | 1 | 500 | 22.03 | 369 | 27.93 |
| C | 2 | 633 | 18.47 | 384 | 28.02 |
| D | 3 | 766 | 15.19 | 370 | 28.05 |

### Example 22 Lidocaine Release

HA and HA-DVS-MBA (83.3% substitution) gels were prepared in a similar manner as described in Example 18 using 0.75% BDDE and a pH of 13.1. About 2 g of each gel were added to a vial and 105 uL of a 6% (w/w) lidocaine HCl in PBS solution was added to each sample. After mixing, the samples were autoclaved for 5 minutes at 121° C. Approx. 1.5 g of each gel was transferred to dialysis tubing (Aldon Corporation, IS13027, 6.4 mm×10 mm, cut-off: 12-14 kDa). A control of lidocaine only in dialysis tubing without gel was also included. The loaded dialysis tubing was placed in separate 45 ml PBS solutions and incubated at 37° C. under oscillating conditions. One (1) ml aliquots were taken at timepoints of 0.5 h, 1 h, 1.5 h, 2.5 h, 4 h, and 7 h. The lidocaine content was measured using HPLC. The percent release was determined by released amount divided by the initial amount added. The release of the lidocaine from the HA and HA-MBA gels was similar. Table 24 shows that the HA-DVS-MBA crosslinked gels has a similar release profile to that of gels prepared from unmodified gels. The lidocaine release from the HA-DVS-MBA gel was slower than the natural dissolution of lidocaine.

TABLE 24

| | Cumulative Release (%) | | |
|---|---|---|---|
| Time | Lidocaine | HA | MBA |

| (hr) | only | AVG | STD | Avg | STD |
|---|---|---|---|---|---|
| 0.5 | 82 | 33 | 5 | 32 | 12 |
| 1 | 87 | 50 | 4 | 47 | 2 |
| 1.5 | 86 | 62 | 5 | 59 | 1 |
| 2 | 84 | 69 | 6 | 67 | 2 |
| 3.5 | 82 | 82 | 5 | 80 | 2 |

### Example 23 In Vivo Persistence

The HA and HA-DVS-MBA (substitution 80.6%) gels were prepared in a similar manner as described in example 10 using a pH of 13.1. Each gel was added to a syringe that was then connected to a second syringe through a luer connector. The gels were passed back and forth between syringes 20 times. The gels were then autoclaved at 121° C. for 5 minutes. Each rat was anesthetized by inhalation anesthesia of 1-4% isoflurane in oxygen. Each rat was shaved in the dorsal region and the skin as wiped with an antiseptic agent. A subcutaneous injection of 150 μL of the 1% BDDE crosslinked HA-MBA gel was injected paraspinally along the left dorsum towards the head of each rat. A second subcutaneous injection of 150 μL of a BDDE crosslinked HA-MBA gel was injected paraspinally along the left dorsum towards the tail of each rat. The process was repeated using 150 μL of a 1% BDDE crosslinked HA gel. Each sample was injected using a sterile syringe assembled with a 27G needle. A total of 12 rats were treated. The length, width and height of the formed blebs were measured at various time points using a caliper. The volume of each bleb was calculated using as an ellipsoid volume [(4/3 Π)(1/2 height)(1/2 length)(1/2 width)]. The volume ratio was calculated as the ratio of the volume of the bleb at a specific time to that following implantation. The blebs were measured at 0, 1, 2, 4, 6, 8, 12 and 16 weeks. At 4 weeks, 6 rats were euthanized, the bleb and surrounding tissue was excised, stored in 10% formalin, prepared into histology slides that were then analyzed. At 16 weeks the remaining 6 rats were euthanized the bleb and surrounding tissue was excised, stored in 10% formalin, prepared into histology slides that were then analyzed. The volume of the bleb and the volume ratio for the crosslinked HA-DVS-MBA composition was statistically greater than the crosslinked HA samples at 16 weeks. This confirms than crosslinked HA-DVS-MBA has a greater in-vivo resistance to degradation to HA crosslinked under similar conditions. Histological analysis showed both HA and HA-MBA gels resulted in minimal overall host response which indicates good biocompatibility in the rat at the local level (skin) over the time course of 16 weeks. Table 25 shows the average volume of the blebs for the HA crosslinked gels and the HA-DVS-MBA gels as a function of time. At various time points the volume of the bleb for the crosslinked-HA-DVS-MBA is greater than that for the crosslinked HA. This shows that the crosslinked HA-DVS-MBA composition has a longer in vivo persistence as compared to the crosslinked HA composition. Table 26 shows the volume ratio of the blebs as a function of time. The volume ratio is calculated by dividing the bleb volume as a specific time by the volume of the bleb when initial implanted. Table 26 shows that the volume ratio of the crosslinked HA-DVS-MBA composition is greater than that of the crosslinked HA composition at the later time points which shows that in-vivo, the crosslinked HA-DVS-MBA composition degrades at a slower rate than the crosslinked HA composition. At the 16 week time point, the crosslinked HA-DVS-MBA composition has about the same volume as when the composition was first injected, while the crosslinked HA composition has about half of the volume of the composition when first injected.

| | Table 25 | | | | Table 26 | | | |
|---|---|---|---|---|---|---|---|---|
| | Volume (mm³) | | | | Volume ratio | | | |
| Time | HA | | HA-DVS-MBA | | HA | | HA-DVS-MBA | |
| (weeks) | Avg | STD | Avg | STD | Avg | STD | Avg | STD |
| 0 | 146.7 | 58.2 | 163.7 | 73.9 | 1 | 0 | 1 | 0 |
| 1 | 210.7 | 41.0 | 229.5 | 81.1 | 1.50 | 0.56 | 1.65 | 0.76 |
| 2 | 180.0 | 61.1 | 213.2 | 64.0 | 1.38 | 0.46 | 1.55 | 0.68 |
| 4 | 158.2 | 59.1 | 208.1 | 38.5 | 1.11 | 0.42 | 1.54 | 0.82 |
| 6 | 141.3 | 49.9 | 176.5 | 49.5 | 0.98 | 0.28 | 1.28 | 0.69 |
| 8 | 143.5 | 30.3 | 153.9 | 34.2 | 1.02 | 0.37 | 1.19 | 0.72 |
| 12 | 97.1 | 39.4 | 158.6 | 23.2 | 0.67 | 0.20 | 1.18 | 0.60 |
| 16 | 73.1 | 46.4 | 126.3 | 27.4 | 0.51 | 0.23 | 0.97 | 0.64 |

### Example 24 Crosslinking with PEG-Dithiol

About 0.5 g of the HA-DVS-MBA composition (as made in a similar manner to example 4) [Substitution 81.5%] was weighed out into a 20 mL glass scintillation vial. About 3.5 g 1% NaOH (pH adjusted to 9.5) was added to the vial. The vial was capped and placed on a roller mill for about 3 hrs. The pH was measured and adjusted to 9.5 using 1% NaOH and 2% HCl. About 9 mg Peg-dithiol (3400) was added to the vial and the contents were mixed with a spatula for about 10 min. The vials were capped and placed in an oven (about 50° C.) for about 24 hrs. The gels were removed from the vials and washed several times with PBS until the gel pH was about 7 to 7.5. The process was repeated using a pH of about 8.1.

TABLE 27

| | | | Concentration |
|---|---|---|---|
| Sample | Reaction pH | % Swelling | (mg/mL) |
| 760-058A | 9.5 | 291 | 33.7 |
| 760-058C | 8.1 | 317 | 32.6 |

### Example 25 Preparation of Crosslinked Gels

A series of gels (gel 1-4) were prepared using about 0.95% BDDE as the crosslinker, a reaction temperature of 50° C. and a reaction time of 3 hours in a similar manner as described in Example 10. The formed gels underwent 4 washing steps. Each wash was performed at room temperature using an orbital shaker. Wash 1: 200 mL PBS (pH 7.0) for about 1 hr, 200 mL PBS (pH 7.2) for about 1 hr, 200 mL PBS (pH 7.4) for about 1 hr, 400 mL PBS (pH 7.4) for about 16 hrs and 200 mL PBS (pH 7.4) for about 1 hr. Wash 2: PBS (7.4 pH) at a ratio of 20:1 PBS:gel (v/m) for about 15 hr. Wash 3: PBS (7.4 pH) at a ratio of 20:1 PBS:gel (v/m) for about 25 hr. Wash 4: PBS (7.4 pH) at a ratio of 20:1 PBS:gel (v/m) for about 24 hr. Once the percent swelling was less than about 5%, the gels were considered fully swollen. Table 28 shows the swelling between each washing step. The greatest amount of swelling occurs during the first washing, after which the gels gradually approach equilibrium with minimal additional swelling.

TABLE 28

| | | Initial | Reac- | % swelling from previous washing | | | |
|---|---|---|---|---|---|---|---|
| | | Conc | tion | Initial | | | |
| Gel | Material | (mg/mL) | pH | to 1 | 1 to 2 | 2 to 3 | 3 to 4 |
| 1 | HA-MBA | 87.7 | 12.40 | 256.8 | 34.2 | −0.6 | 4.2 |
| 2 | HA-MBA | 87.9 | 12.27 | 290.2 | 44.8 | 7.0 | 2.4 |
| 3 | HA-MBA | 88.1 | 12.25 | 295.1 | 30.0 | 4.9 | 3.2 |
| 4 | HA | 147.2 | 12.65 | 747.2 | 70.3 | 21.6 | 11.0 |

### Example 26 Degradation of Gels

A portion of the gels, as prepared in example 25 and after wash 4, were weighed and then each gel was incubated in 20 mL PBS solution containing 2 IU/mL hyaluronidase (bovine testes). The mass of the gels used resulted in each test sample having about 19.5 mg to about 24.5 mg HA based material. The samples were incubated for a specific period of time at about 37° C. The mass of the gel was measured after each timepoint. The hyaluronidase solution was replaced with fresh solution. The change in gel mass was calculated. The percent change in gel mass was calculated by dividing the mass of the gel as a specific timepoint by the starting mass of the gel. Table 29 shows that the degradation profile of the HA-DVS-MBA relative to the HA gels for gels at about equilibrium swelling. The HA-DVS-MBA gels degrade slower than the HA gel.

TABLE 29

| | | Change in Mass relative to T = 0 (%) | | |
|---|---|---|---|---|
| Gel | Material | 6 h | 24 h | 48 h |
| 1 | HA-DVS-MBA | 3.05 | −1.81 | −12.81 |
| 2 | HA-DVS-MBA | 1.00 | −20.97 | −47.21 |
| 3 | HA-DVS-MBA | 10.93 | −6.12 | −32.70 |
| 4 | HA | −15.81 | −63.90 | −91.82 |

### Example 27 Rehydration with Biologically Active Agent

Crosslinked compositions made according to examples 9, 10, 11, 13, 14, 15, 16, 17, 18, 21 or 23 are dried and or lyophilized. The lyophilized composition is rehydrated with either BMP-7 (5 μg/mL) or Botox (5 U/mL). The resultant gel composition is applied onto or into a tissue of a subject.

### Example 28 Rehydration with a Chemotherapeutic Agent

Crosslinked compositions made according to examples 9, 10, 11, 13, 14, 15, 16, 17, 18, 21 or 23 can be dried and or lyophilized. The lyophilized composition is rehydrated with either doxorubicin (2 mg/mL), cisplatin (1 mg/mL), gemcitabine (100 mg/mL), epirubicin (2 mg/mL) or oxaliplatin (5 mg/mL). The resultant gel composition is applied onto or into a tissue of a subject.

### Example 29 Incubation with a Chemotherapeutic Agent

Crosslinked compositions made according to examples 9, 10, 11, 13, 14, 15, 16, 17, 18, 21 or 23 can be incubated in the presence of a chemotherapeutic agent. The formed gel composition is added to a solution of either doxorubicin (2 mg/mL), cisplatin (1 mg/mL), gemcitabine (100 mg/mL), epirubicin (2 mg/mL) or oxaliplatin (5 mg/mL). After about 48 hr in the dark, the gel composition is separated from the remaining solution. The resultant gel composition is applied onto or into a tissue of a subject.

### Example 30 Calcium Washing

The gel composition is prepared according to example 9, 10, 11, 13, 14, 15, 16, 17, 18, 21 or 23. After the final washing the gel is broken up into smaller particles by passing the gel through a 300 μm mesh screen about three times. One gram of the gel particles are filtered and immersed in acetone for 30 minutes. The particles are filtered and air dried. The particles are added to a calcium chloride dihydrate solution (0.15M, pH 3). After 30 min, the pH of the solution is increased to pH 7.4. After 30 min, the particles are filtered and equilibrated in PBS for 2 hrs. The particles were filtered and transferred to a 2 mL syringe.

### Example 31 Calcium Phosphate Washing

The gel composition is prepared according to example 9, 10, 11, 13, 14, 15, 16, 17, 18, 21 or 23. After the final washing the gel is broken up into smaller particles by passing the gel through a 300 μm mesh screen about three times. One gram of the gel particles are filtered and immersed in acetone for 30 minutes. The particles are filtered and air dried. The particles are added to a solution comprising calcium chloride dihydrate solution (0.15M) and phosphoric acid (0.09M). After 60 min, the particles are filtered and added to a 10% (v/v) ammonium hydroxide solution. After 30 min, the particles are filtered and equilibrated in PBS for 2 hrs. The particles were filtered and transferred to a 2 mL syringe.

All references disclosed herein, including patent references and non-patent references, are hereby incorporated by reference in their entirety as if each was incorporated individually.

It is to be understood that the terminology used herein is for the purpose of describing specific aspects only and is not intended to be limiting. It is further to be understood that unless specifically defined herein, the terminology used herein is to be given its traditional meaning as known in the relevant art.

Reference throughout this specification to “an aspect” or “an aspect” and variations thereof means that a particular feature, structure, or characteristic described in connection with the aspect is included in at least an aspect. Thus, the appearances of the phrases “in an aspect” or “in an aspect” in various places throughout this specification are not necessarily all referring to the same aspect. Furthermore, the particular features, structures, or characteristics may be combined in any suitable manner in one or more aspects.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an aspect that includes all of the associated items or ideas and one or more other alternative aspects that include fewer than all of the associated items or ideas.

Unless the context requires otherwise, throughout the specification and claims that follow, the word “comprise” and synonyms and variants thereof such as “have” and “include,” as well as variations thereof such as “comprises” and “comprising” are to be construed in an open, inclusive sense, e.g., “including, but not limited to.” The term “consisting essentially of” limits the scope of a claim to the specified compositions or steps, or to those that do not materially affect the basic and novel characteristics of the claimed disclosure.

Any headings used within this document are only being utilized to expedite its review by the reader, and should not be construed as limiting the disclosure or claims in any manner. Thus, the headings and Abstract of the Disclosure provided herein are for convenience only and do not interpret the scope or meaning of the aspects.

Where a range of values is provided herein, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, compositions and methodologies described in the publications, which might be used in connection with the presently described disclosure. The publications discussed above and throughout the text are provided solely for their disclosure prior to the filing date of the present application. Nothing herein is to be construed as an admission that the inventors are not entitled to antedate any referenced publication by virtue of prior disclosure.

All patents, publications, scientific articles, web sites, and other documents and compositions referenced or mentioned herein are indicative of the levels of skill of those skilled in the art to which the disclosure pertains, and each such referenced document and composition is hereby incorporated by reference to the same extent as if it had been incorporated by reference in its entirety individually or set forth herein in its entirety. Applicants reserve the right to physically incorporate into this specification any and all compositions and information from any such patents, publications, scientific articles, web sites, electronically available information, and other referenced compositions or documents.

In general, in the following claims, the terms used should not be construed to limit the claims to the specific aspects disclosed in the specification and the claims, but should be construed to include all possible aspects along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting aspects are within the following claims. The patent may not be interpreted to be limited to the specific examples or nonlimiting aspects or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A derivative of hyaluronic acid in which one or more hydroxyl groups of the hyaluronic acid is a modified hydroxyl group, wherein the derivative of hyaluronic acid has the structure HA-(OCH₂CH₂SO₂CH₂CH₂—X—R¹)ₙ where HA is hyaluronic acid, X is S or NH, R¹ is a substituted or unsubstituted aromatic moiety and n is the number of modified hydroxyl groups where n≥1 and wherein the in-vitro hyaluronidase degradation rate is slower than unmodified hyaluronic acid.

2. A derivative of hyaluronic acid in which two or more hydroxyl groups of the hyaluronic acid are modified hydroxyl groups, wherein the derivative of hyaluronic acid has the structure (R²—X—CH₂CH₂SO₂CH₂CH₂O)ₘ—HA-(OCH₂CH₂SO₂CH₂CH₂—X—R¹)ₙ where HA is hyaluronic acid, X is S or NH, R¹ is a substituted or unsubstituted aromatic moiety, R² is a substituted or unsubstituted C₁-C₂₀ aliphatic or aromatic moiety wherein R¹ and R² are different from each other, n≥1 and m≥1 and wherein the in-vitro hyaluronidase degradation rate is slower than unmodified hyaluronic acid.

3. A derivative of hyaluronic acid in which two or more hydroxyl groups of the hyaluronic acid are modified hydroxyl groups, wherein the derivative of hyaluronic acid has the structure (CH₂═CH—SO₂CH₂CH₂O)ₘ—HA-(OCH₂CH₂SO₂CH₂CH₂—X—R¹)ₙ where HA is hyaluronic acid, X is S or NH, R¹ is a substituted or unsubstituted aromatic moiety, n≥1 and m≥1 and wherein the in-vitro hyaluronidase degradation rate is slower than unmodified hyaluronic acid.

4. The derivative of hyaluronic acid according to any of claims 1-3 wherein 0.25-50% of a sum of the hydroxyl groups and the modified hydroxyl groups are a modified hydroxyl group.

5. The derivative of hyaluronic acid according to any of claims 1-3 wherein R¹ comprises a carboxylic acid group, or a salt or ester thereof.

6. The derivative of hyaluronic acid according to any of claims 1-3 wherein R¹ comprises a hydroxyl group.

7. The derivative of hyaluronic acid according to any of claims 1-3 wherein R¹ comprises a benzene ring.

8. A crosslinked polymer comprising a reaction product of a derivative of hyaluronic acid and a crosslinking agent, wherein
a) the derivative of hyaluronic acid has the structure HA-(OCH₂CH₂SO₂CH₂CH₂—X—R¹)ₙ wherein one or more hydroxyl groups of the hyaluronic acid is a modified hydroxyl group, and wherein HA is hyaluronic acid comprising hydroxyl groups, X is S or NH, R¹ is a substituted or unsubstituted aromatic moiety and n is the number of modified hydroxyl groups where n≥1;
b) the crosslinking agent comprises at least two functional groups that are capable of reacting with the hydroxyl groups of the derivative of hyaluronic acid; and
c) and the in-vitro hyaluronidase degradation rate is slower than similarly crosslinked unmodified hyaluronic acid.

9. The crosslinked polymer according to claim 8 wherein 0.25-50% of a sum of the hydroxyl groups and the modified hydroxyl groups are a modified hydroxyl group.

10. The crosslinked polymer according to claim 8 wherein R¹ comprises a carboxylic acid group, or a salt or ester thereof.

11. The crosslinked polymer according to claim 8 wherein R¹ comprises a hydroxyl group.

12. The crosslinked polymer according to claim 8 wherein R¹ comprises a benzene ring.

13. A crosslinked polymer comprising a reaction product of a derivative of hyaluronic acid and a crosslinking agent, wherein
a) the derivative of hyaluronic acid comprises vinyl groups and has the structure (CH₂═CH—SO₂CH₂CH₂O)ₘ—HA-(OCH₂CH₂SO₂CH₂CH₂—X—R¹)ₙ wherein two or more hydroxyl groups of the hyaluronic acid are modified hydroxyl groups, HA is hyaluronic acid comprising hydroxyl groups, X is S or NH, R¹ is a substituted or unsubstituted aromatic moiety, n≥1 and m≥1; and
b) the crosslinking agent comprises at least two functional groups that are capable of reacting with the vinyl groups of the derivative of hyaluronic acid.

14. The crosslinked polymer according to claim 13 wherein 0.25-50% of a sum of the hydroxyl groups and the modified hydroxyl groups are a modified hydroxyl group.

15. The crosslinked polymer according to claim 13 wherein R¹ comprises a carboxylic acid group, or a salt or ester thereof.

16. The crosslinked polymer according to claim 13 wherein R¹ comprises a hydroxyl group.

17. The crosslinked polymer according to claim 13 wherein R¹ comprises a benzene ring.

18. A crosslinked polymer comprising a reaction product of a derivative of hyaluronic acid with itself, wherein
a) the derivative of hyaluronic acid comprises vinyl groups and has the structure (CH₂═CH—SO₂CH₂CH₂O)ₘ—HA-(OCH₂CH₂SO₂CH₂CH₂—X—R¹)ₙ wherein two or more hydroxyl groups of the hyaluronic acid are modified hydroxyl groups, HA is hyaluronic acid comprising hydroxyl groups, X is S or NH, R¹ is a substituted or unsubstituted aromatic moiety, n≥1 and m≥1.

19. The crosslinked polymer according to claim 13 wherein 0.25-50% of a sum of the hydroxyl groups and the modified hydroxyl groups are a modified hydroxyl group.

20. The crosslinked polymer according to claim 13 wherein R¹ comprises a carboxylic acid group, or a salt or ester thereof.

21. The crosslinked polymer according to claim 13 wherein R¹ comprises a hydroxyl group.

22. The crosslinked polymer according to claim 13 wherein R¹ comprises a benzene ring.

23. A process comprising:
a) reacting hydroxyl groups attached to a polymer, such as hydroxyl groups on hyaluronic acid (HA), with divinyl sulfone (DVS) to provide a first derivative of the polymer; and
b) reacting the first derivative of the polymer with a nucleophile of a formula selected from X—R¹ and X—R²—Y to provide a second derivative of the polymer;
wherein R¹ is a substituted or unsubstituted aromatic moiety, R² is a substituted or unsubstituted C₁-C₂₀ aliphatic or aromatic moiety, X is a nucleophilic group, and Y is selected from carboxylic acid, sulfonic acid and hydroxyl.

24. The process of claim 23 wherein 0.25-50% of the hydroxyl groups present on the polymer are converted to oxyethyl ethenyl sulfone groups of the formula —OCH₂CH₂—SO₂CH═CH₂.

25. The process of claim 23 wherein the polymer is hyaluronic acid and the first derivative of the polymer is an oxyethyl ethenyl sulfone derivative of the hyaluronic acid=HA-OCH₂CH₂SO₂CH═CH₂ (HA-DVS).

26. The process of claim 23 wherein the second derivative is HA-OCH₂CH₂SO₂CH₂CH₂—X′—R¹ (HA-DVS-N).

27. The process of claim 23 wherein 0.25-50% of the hydroxyl groups present on the polymer are converted to —OCH₂CH₂SO₂CH₂CH₂—X′—R¹ groups.

28. The process of claim 23 wherein X is thiol and X′ is —S—.

29. The process of claim 23 wherein the second derivative is HA-OCH₂CH₂SO₂CH₂CH₂—X′—R²—Y (HA-DVS-NY).

30. The process of claim 29 wherein 1-50% of the hydroxyl groups present on the polymer are converted to —OCH₂CH₂SO₂CH₂CH₂—X′—R²—Y groups.

31. The process of claim 30 wherein X is thiol and X′ is —S—.

32. The process of claim 30 wherein Y is hydroxyl.

33. The process of claim 30 wherein Y is carboxylic acid or a salt or ester thereof.

34. The process of claim 30 wherein Y is sulfonic acid of a salt of ester thereof.

35. The process of claim 30 further comprising reacting the second derivative of the polymer with a crosslinking agent to provide a third derivative of the polymer, where the third derivative is a crosslinked polymer.

36. A derivative of hyaluronic acid prepared by the process of any of claims 23-35.

37. A crosslinked polymer prepared by the process of claim 35.

38. A composition comprising a derivative of hyaluronic acid according to any of claims 1-7 and an excipient.

39. A composition comprising a crosslinked polymer according to any of claims 8-22 and an excipient.

40. A composition comprising a derivative of hyaluronic acid and an excipient, wherein the derivative of hyaluronic acid is prepared according to process of any of claims 23-34.

41. A composition comprising a crosslinked polymer and an excipient, wherein the crosslinked polymer is prepared according to the process of claim 35.

42. A composition according to any of claims 37-41 wherein the excipient is selected from a synthetic polymer, thermosreversible polymer, biodegradable polymer, hyaluronic acid or a salt thereof, buffer, complexing agent, tonicity modulator, ionic strength modifier, solvent, anti-oxidant, preservative, viscosity modifier, pH modifier, surfactant, emulsifier, phospholipid, stabilizer and porogen.

43. A composition according to any of claims 37-42 further comprising a biologically active agent.

44. A method for wound healing comprising administering to a subject in need thereof an effective amount of a composition according to any of claims 38-43.

45. A bulking agent comprising a composition according to any of claims 37-42.

46. A dermal filler comprising a composition according to any of claims 37-42.

47. A method of filling a void in a subject in need thereof comprising administering to the subject a dermal filler according to claim 46.

48. A viscosupplement comprising a composition according to any of claims 37-42.

49. A method of relieving joint pain in a subject in need thereof, comprising administering to the subject a viscosupplement according to claim 48.

50. A method of preventing surgical adhesions in a subject in need thereof comprising administering the subject an effective amount of a composition according to any of claims 37-42.

51. A tissue sealant comprising a composition according to any of claims 37-42.

52. A method of sealing tissue in a subject in need thereof comprising administering to the subject an effective amount of a tissue sealant according to claim 51.

53. A method of treating Bacterial vaginosis in a subject in need thereof comprising administering to the subject an effective amount of a composition according to any of claims 37-42.

54. An eye drop comprising a composition according to any of claims 37-42.

55. A method of treating an ocular condition in a subject in need thereof comprising administering the subject an effective amount of a composition according to any of claims 37-42.

56. A punctal plug comprising a composition according to any of claims 37-42.

57. A method of treating mucositis in a subject in need thereof comprising administering to the subject an effective amount of a composition according to any of claims 37-42.

58. An anti-bacterial formulation comprising a composition according to any of claims 37-42.

59. A method of treating an ear condition comprising administering to a subject in need thereof an effective amount of a composition according to any of claims 37-42.

60. A method of drug delivery to a subject in need thereof comprising administering to the subject an effective amount of a composition according to any of claims 37-42 that comprises the drug.

61. A biopsy plug comprising a composition according to any of claims 37-42.

62. A plug for female sterilization comprising a composition according to any of claims 37-42.

63. A tissue scaffold comprising a composition according to any of claims 37-42.

64. The method of supporting tissue growth in a subject in need thereof comprising implanting in the subject a tissue scaffold according to claim 63.

65. A burr hole plug comprising a composition according to any of claims 37-42.

66. A nerve guide comprising a composition according to any of claims 37-42.

67. A vaginal lubricant comprising a composition according to any of claims 37-42.

68. A coating for a device comprising a composition according to any of claims 37-42.

69. A method for coating a device comprising applying a coating of claim 68 onto a surface of the device.

70. A method for additive manufacturing comprising melting a solid derivative of hyaluronic acid according to any of claims 1-7, or prepared by a process according to any of claims 23-35 to provide a molten derivative of hyaluronic acid, and depositing the molten derivative of hyaluronic acid onto a substrate to provide an article formed by additive manufacturing.

71. A crosslinked gel comprising a derivatized hyaluronic acid wherein, the derivative of hyaluronic acid comprises the structure HA-(OCH₂CH₂SO₂CH₂CH₂—X—R¹)ₙ where HA is hyaluronic acid, X is S or NH, R¹ is a substituted or unsubstituted aromatic moiety and n is the number of modified hydroxyl groups where n≥1.

72. A crosslinked gel according to claim 71, wherein R1 is a AR—COOH group, where AR is a benzene ring.

73. A crosslinked gel according to claim 71, wherein R1 is a benzene ring.

74. A crosslinked gel according to claims 71-73 wherein the crosslinker used is 1,4-butanediol diglycidyl ether (BDDE).

75. A crosslinked gel according to claims 71-74 wherein the in-vitro hyaluronidase degradation rate is slower than that of a similarly crosslinked non-derivatized hyaluronic acid.

76. A composition comprising a crosslinked gel according to claims 71-74 and unmodified hyaluronic acid or a salt thereof.

77. A dermal filler comprising the composition of any of the claims 71-76.

78. A dermal filler according to claims 71-76 wherein the composition further comprises lidocaine.

79. A dermal filler according to claims 71-77 that can be injected through a 27G needle.

80. A kit comprising a composition according to claims 71-79 and a syringe.

81. A tissue spacer comprising the composition of any of the claims 71-76.

82. A composition comprising a crosslinked gel according to claims 8-22 wherein the composition further comprises hyaluronic acid or a salt thereof, or a derivative of hyaluronic acid according to any of claims 1-7.

83. A composition comprising a crosslinked gel according to claim 82 wherein the composition further comprises lidocaine.
